SEQUENCE LISTING (61 sequences)

SEQ ID NO: 1 - DNA, 874 bp, Pisum sativum
cgcaacctagcttttcctccataatcgattattccattcatttcaatggccttgcctaatcagcaaacagttgattatcctagtttcaagcttgtgatcgtcggtgacggtggcacaggaaaaacaactttcgtaaagaggcatcttactggagaatttgagaagaaatatgaaccaactattggtgttgaagtgcatccattggatttcttcacaaagtgtggtaagattcgtttctactgctgggatactgctggtcaagagaagtttggtggtctcagagatggatactatattcatggacagtgtgctattataatgtttgatgttactgcacggctgacatacaagaatgtccctacttggcaccgtgatctttgccgggtctgtgaaaacatcccaattgttctttgtggaaacaaggttgatgtgaagaacaggcaagtgaaggcaaagcaggttaccttccacaggaagaaaaatttgcagtactatgaaatatcagctaagagcaactacaactttgagaagcctttcctgtatcttgccaggaaacttgcaggtgatcctaatttgcactttgtagaatctcctgcactggctccaccagaagttcaaattgatattgctttacaacaaaggcatgagaatgagattcttgaggctgctaatcagccccttcctgatgatgatgacgatgcttttgaataggaaggaccttcgagagtgctggcctatgtgtgttgatttgtgccgttatatgctgtaatgttatgtgttttgtggttgaaccgttgacatttttttgatatagatttatggatttcaatcatcatgtgattgagttgttttaaaaaaaaaaaaaaaaaaaaaa

SEQ ID NO: 2 - DNA, 1075 bp, Arabidopsis thaliana
gaaaccaactcagagcaaaagcttgatttgacactatggcgagcattagcaacgagcccgagcgtgagaacagagacgaagaagagactggagccaacgaagatgaagacaccggtgctcaggttgctcctatcgtcaggcttgaagaagtcgccgtcaccaccggtgaagaagacgaagacaccatcctcgatctgaaatcgaagttgtatcgatttgataaagatggaagtcagtggaaggagagaggtgctggtactgttaagtttgtgaaacatagagtttctgggaagattcgtctcgttatgaggcaatcgaaaactttgaagatctgtgctaatcatcttgttggatcgggtatgagtgttcaggaacacgctgggaatgataagtcttgtgtatggcacgctcgtgatttctccgatggtgaattgaaggatgagctcttctgtatccggtttggttcagttgagaattgcaaagcatttatgcaaaagttcaaggaagtagctgaatctgaagaagagaaagaagagagcaaagatgcctctgataccgctggtcttcttgagaagttaacagtggaagagaaggaaagtgagaagaaaccagtggagaaggcagaggaaaacaaaaagagtgaagctgttgaagaaaagaaaacagaggagtctgttccctcagcttaagatgcatgcttacacacacatacggccaggtaatcaatagcctgatgtgttttaaatccatcgaaggaagacttggcttttattttcaaattgaatacatgttttgagtcgtgttgagagtctatatttttagtgtcacaaaagtgtcacaggtgacattttgcatatgcgagattgattttcgagcttgtttgggtttcattatctggaagaaaaaaagagtgtattttgagctctcttatgatttttttttggaaaaagtgtggactcaagagaagattttattttgagttcacaaaaatttgtatgcatggatgattcgcgtgttctctttttaatgccagtcgtgttattgaatcaaaaaaaaaaaaaaaaaaaaaaaaaaa

SEQ ID NO: 3 - DNA, 962 bp, Arabidopsis thaliana
gtcgacccacgcgtccggaagttgcagagcacacacacacactttcaagtttcaatacagagatggcgagcactgagccagagcgtgagaaccgtgaagatgaaaccgaagtcaacgaagatgaagacaccggagctcaggtagctccgatcgttaggcttgaagaggttgcagtcaccaccggcgaagaagatgaagacgccgtcctcgatctgaaatcgaagatgtatcgattcgataaagaagggaaccaatggaaggagagaggagctggaactgtgaagttattgaagcataaggaaactggaaaagttcgtcttgttatgagacaatctaaaaccctaaagatctgtgccaatcacctcatttcgtctgggatgagtgttcaggaacatagtgggaatgaaaagtcttgtttatggcacgctactgatttctccgacggcgagttgaaagatgagcttttctgcattcgatttgcttccattgagaattgcaaaacatttatggagaagttcactgaaatagctgaaagccagcaagtagggaaagagagcacccagggcgacgaggctgctggtttaatagagaatctttcggttgaggaaaatataagtgaggagaaagccaaggaagcagaagagaaagagcctgcaaaggaagataaagaaacaaaaaaggagaaggtagaagaagagaagaaaacagaggcaagcacttaagtgatcaaattaaaattggttgttggcatcactaagtgtgctttgctttattttaatatattatacttggcactgaacatgatggtagcttcaaaagtttgagtctttgcgctgtgttagagaaagagaatcttaagatttttttttatgttgttgctgtgtaagtgtttgactattgtgtcgtctctttactgtgtcaatttggtttaagataataaaaaaaaaaaaaaaaaaaaaaaa

SEQ ID NO: 4 - DNA, 1073 bp, Arabidopsis thaliana
tttttttttttttttttttttttttgattcaataacacgactggcattaaaaagagaacacgcgaatcatccatgcatacaaatttttgtgaactcaaaataaaatcttctcttgagtccacactttttccaaaaaaaaatcataagagagctcaaaatacactctttttttcttccagataatgaaacccaaacaagctcgaaaatcaatctcgcatatgcaaaatgtcacctgtgacacttttgtgacactaaaaatatagactctcaacacgactcaaaacatgtattcaatttgaaaataaaagccaagtcttccttcgatggatttaaaacacatcaggctattgattacctggccgtatgtgtgtgtaagcatgcatcttaagctgagggaacagactcctctgttttcttttcttcaacagcttcactctttttgttttcctctgccttctccactggtttcttctcactttccttctcttccactgttaacttctcaagaagaccagcggtatcagaggcatctttgctctcttctttctcttcttcagattcagctacttccttgaacttttgcataaatgctttgcaattctcaactgaaccaaaccggatacagaagagctcatccttcaattcaccatcggagaaatcacgagcgtgccatacacaagacttatcattcccagcgtgttcctgaacactcatacccgatccaacaagatgattagcacagatcttcaaagttttcgattgcctcataacgagacgaatcttcccagaaactctatgtttcacaaacttaacagtaccagcacctctctccttccactgacttccatctttatcaaatcgatacaacttcgatttcagatcgaggatggtgtcttcgtcttcttcaccggtggtgacggcgacttcttcaagcctgacgataggagcaacctgagcaccggtgtcttcatcttcgttggctccagtctcttcttcgtctctgttctcacgctcgggctcgttgctaatgctcgccatagtgtcaaatcaagcttttgctctgagttggtttc

SEQ ID NO: 5 - DNA, 923 bp, Arabidopsis thaliana
ttttttttttttttttttttttttattatcttaaaccaaattgacacagtaaagagacgacacaatagtcaaacacttacacagcaacaacataaaaaaaaatcttaagattctctttctctaacacagcgcaaagactcaaacttttgaagctaccatcatgttcagtgccaagtataatatattaaaataaagcaaagcacacttagtgatgccaacaaccaattttaatttgatcacttaagtgcttgcctctgttttcttctcttcttctaccttctccttttttgtttctttatcttcctttgcaggctctttctcttctgcttccttggctttctcctcacttatattttcctcaaccgaaagattctctattaaaccagcagcctcgtcgccctgggtgctctctttccctacttgctggctttcagctatttcagtgaacttctccataaatgttttgcaattctcaatggaagcaaatcgaatgcagaaaagctcatctttcaactcgccgtcggagaaatcagtagcgtgccataaacaagacttttcattcccactatgttcctgaacactcatcccagacgaaatgaggtgattggcacagatctttagggttttagattgtctcataacaagacgaacttttccagtctctccttccattggttcccttctttatcgaatcgatacatcttcgatttcagatcgaggacggcgtcttcatcttcttcgccggtggtgactgcaacctcttcaagcctaacgatcggagctacctgagctccggtgtcttcatcttcgttgacttcggtttcatcttcacggttctcacgctctggctcagtgctcgccatctctgtattgaaacttgaaagtgtgtgtgtgtgctctgcaacttccggacgcgtgggtcg

SEQ ID NO: 6 - DNA, 26 bp, Artificial
  sense primer for PsRan
attcgaattcatggccttgcctaatc

SEQ ID NO: 7 - DNA, 27 bp, Artificial
  antisense primer for PsRan
cgcgggatcctctgaaggtccttccta

SEQ ID NO: 8 - DNA, 31 bp, Artificial
  sense primer for AtRanBP1b
tttctcgagatggcgagcattagcaacgagc

SEQ ID NO: 9 - DNA, 32 bp, Artificial
  antisense primer for AtRanBP1b
aaatctagaacacgcgaatcatccatgcatac

SEQ ID NO: 10 - DNA, 30 bp, Artificial
  sense primer for AtRanBP1c
aatctcgagatggcgagcactgagccagag

SEQ ID NO: 11 - DNA, 31 bp, Artificial
  antisense primer for AtRanBP1c
aaatctagacagtaaagagacgacacaatag

SEQ ID NO: 12 - DNA, 29 bp, Artificial
  RT-PCR primer for antisense AtRanBP1b
cacctcgagtcttccagataatgaaaccc

SEQ ID NO: 13 - DNA, 28 bp, Artificial
  RT-PCR primer for antisense AtRanBP1b
ttttctagaatggcgagcattagcaacg

SEQ ID NO: 14 - DNA, 28 bp, Artificial
  RT-PCR primer for antisense AtRanBP1c
ctgctcgagtgctttctcctcacttata

SEQ ID NO: 15 - DNA, 29 bp, Artificial
  RT-PCR primer for antisense AtRanBP1c
gcgtctagaccgtgaagatgaaaccgaag

SEQ ID NO: 16 - DNA, 657 bp, Glycine max
atgtcgagcgccgatcccgagcacagagaagaggaggaggcacccgccgttggcgacgacgaggacaccggagctcacgtcgctccaatcgtcaagctcgaggaggttgctgtcaccaccggcgaggaggacgaagatgcaatcctggatctcaaatcgaagctttaccggttcgacaaggacgggaaccagtggaaggagcgtggcgccggcactgtgaagttcctgaagcataaggcttccggcaaggttaggcttctcatgaggcaatccaagacgctcaagatctgcgctaatcatctcattttgcctacgatgagtgtgcaagagcacgctgggaacgagaaatcctgcgtttggcatgctagggattttgctgatggtgaactcaaggacgagctcttctgcatccgttttccttctattgaaaattgcaaaagcttcatggaaacattccaagaagttgctgaaacacaaaaggcagaggatgagaaggaatcatctgatgcagctggtcttattgagaaattaagtgttgaagagaaggctgatgcagagaagaaagatgaagagaagtctgaggacaaaactgtggagaaagaatctgcatcaggaaaggaaagcaaggcagttgaagagcctgcgtcctcagcttga

SEQ ID NO: 17 - DNA, 657 bp, Glycine max
tcaagctgaggacgcaggctcttcaactgccttgctttcctttcctgatgcagattctttctccacagttttgtcctcagacttctcttcatctttcttctctgcatcagccttctcttcaacacttaatttctcaataagaccagctgcatcagatgattccttctcatcctctgccttttgtgtttcagcaacttcttggaatgtttccatgaagcttttgcaattttcaatagaaggaaaacggatgcagaagagctcgtccttgagttcaccatcagcaaaatccctagcatgccaaacgcaggatttctcgttcccagcgtgctcttgcacactcatcgtaggcaaaatgagatgattagcgcagatcttgagcgtcttggattgcctcatgagaagcctaaccttgccggaagccttatgcttcaggaacttcacagtgccggcgccacgctccttccactggttcccgtccttgtcgaaccggtaaagcttcgatttgagatccaggattgcatcttcgtcctcctcgccggtggtgacagcaacctcctcgagcttgacgattggagcgacgtgagctccggtgtcctcgtcgtcgccaacggcgggtgcctcctcctcttctctgtgctcgggatcggcgctcgacat

SEQ ID NO: 18 - DNA, 351 bp, Glycine max
ccaagaattcggcaccagcacacacaaaccctcccctcacactctccaaaccctagcggcactacacacgttatctctctctctctctctctctctccctctctccttccccaagatgtcgagcgccgatcccgagcaccgagaagaggaggaggcacccgccgtcggcgacgacgaggacaccggagcacacgtcgctcccatcgtcaagctcgaggaggttgctgtcaccaccggcgaggaggacgaagatgcaatcctagatctcaaatcgaagctttaccggttcgacaaggatgggaaccagtggaaggagcgaggcgcgggcaccgtgaagttcctgaagcataa

SEQ ID NO: 19 - DNA, 351 bp, Glycine max
ttatgcttcaggaacttcacggtgcccgcgcctcgctccttccactggttcccatccttgtcgaaccggtaaagcttcgatttgagatctaggattgcatcttcgtcctcctcgccggtggtgacagcaacctcctcgagcttgacgatgggagcgacgtgtgctccggtgtcctcgtcgtcgccgacggcgggtgcctcctcctcttctcggtgctcgggatcggcgctcgacatcttggggaaggagagagggagagagagagagagagagagagataacgtgtgtagtgccgctagggtttggagagtgtgaggggagggtttgtgtgtgctggtgccgaattcttgg

SEQ ID NO: 20 - DNA, 418 bp, Gossypium hirsutum
attcgattgctcaagttgcccctatcgtcaggcttgaggaagtcgccgttaccaccggcgaggaagaagaagatgccatactcgacctgaagtcgaaactgtaccgatttgataaagacgggaatcaatggaaagagagaggcgctggtactgtgaagctgttgaagcacaaaaagactgggaaagttcgccttgttatgaggcaatctaagactctcaagatctgcgccaatcattatgtgttggcaacgatgacggtgcaggagcacgcaggaaacgacaaatcgtgcctatggcacgcttctgactttgctgatgatgaactgaaagatgaacttttctgcattcgttttgcgtcagtggaaaattgcaaaagcttcatggaaacggtccgagaagttgctgaataatcactagt

SEQ ID NO: 21 - DNA, 418 bp, Gossypium hirsutum
actagtgattattcagcaacttctcggaccgtttccatgaagcttttgcaattttccactgacgcaaaacgaatgcagaaaagttcatctttcagttcatcatcagcaaagtcagaagcgtgccataggcacgatttgtcgtttcctgcgtgctcctgcaccgtcatcgttgccaacacataatgattggcgcagatcttgagagtcttagattgcctcataacaaggcgaactttcccagtctttttgtgcttcaacagcttcacagtaccagcgcctctctctttccattgattcccgtctttatcaaatcggtacagtttcgacttcaggtcgagtatggcatcttcttcttcctcgccggtggtaacggcgacttcctcaagcctgacgataggggcaacttgagcaatcgaat

SEQ ID NO: 22 - DNA, 523 bp, Gossypium hirsutum
cgatttgctcaagtagctcctatcgtcaagctcgaggaagtcgctgtcactactggtgaagaagacgaggatccgattctcgatctgaaggcaaagctgtatcgattcgataaggaagggaatcaatggaaggagagaggagttggaaatgtgaagctcttgaagcacaaagtcaatggcaaagtccgtctagtcatgcgccaatctaaaacccttaagatctgtgctaaccatttcgttattccaacaatgacggtgcaagaacattctggaaatgataaatcatgcgtttggcacgccgctgatttcgctgatgggaatttgaaagaagagatgttctgtatcagatttgcttctgttgagaattgtaagaccttcatggagacagttcaagatgttgctgaaagtcagggaaagaaagaggaaaataaagacgctacagtcactgctgatctcctcgacaagttgagtgttggagaaagtaaaagtgacggtaaagaagaagatacggtcgaatcactag

SEQ ID NO: 23 - DNA, 523 bp, Gossypium hirsutum
ctagtgattcgaccgtatcttcttctttaccgtcacttttactttctccaacactcaacttgtcgaggagatcagcagtgactgtagcgtctttattttcctctttctttccctgactttcagcaacatcttgaactgtctccatgaaggtcttacaattctcaacagaagcaaatctgatacagaacatctcttctttcaaattcccatcagcgaaatcagcggcgtgccaaacgcatgatttatcatttccagaatgttcttgcaccgtcattgttggaataacgaaatggttagcacagatcttaagggttttagattggcgcatgactagacggactttgccattgactttgtgcttcaagagcttcacatttccaactcctctctccttccattgattcccttccttatcgaatcgatacagctttgccttcagatcgagaatcggatcctcgtcttcttcaccagtagtgacagcgacttcctcgagcttgacgataggagctacttgagcaaatcg

SEQ ID NO: 24 - DNA, 660 bp, Gossypium hirsutum
atggcaagcaaagaacccgaacacgagcacagagaagatgaagaagctcctgccgccgaggacgaagacactggagctcaagtcgcccctattgtcaagttagaggaagtcgctgtcagtacaggcgaggaaaatgaagatccgatcctcgatctgaagtcgaagctgtatcgatttgataaagaagggaatcactggaaagagagaggtgctggtactgtgaagctgttgaagcacaaagagacaggaaaagttcgccttgttatgaggcaatctaagactctcaagatctgcgccaatcatctagtgttaccgacaatgacggtgcaggagcacgctgggaatgataaatcgtgcttatggcacgcctctgactatgcagatggtgaattgaaagatgaacttttctgcattcgttttgcatcagtggaaaattgcaaaacctttatgcaaatgttccaagaagttgctgaatcacaaaaaccaaaagaggaaaacaaggatgcatctgatgctgctggacaacttgagaagttgagctttcaagagaaagctggagaggagaaggctgttgctaagaaggaagagaaggaaacaaaagtggatacagaaaaagcagatacagagaacaaagatggggagccagcttcctcaacttga

SEQ ID NO: 25 - DNA, 660 bp, Gossypium hirsutum
tcaagttgaggaagctggctccccatctttgttctctgtatctgctttttctgtatccacttttgtttccttctcttccttcttagcaacagccttctcctctccagctttctcttgaaagctcaacttctcaagttgtccagcagcatcagatgcatccttgttttcctcttttggtttttgtgattcagcaacttcttggaacatttgcataaaggttttgcaattttccactgatgcaaaacgaatgcagaaaagttcatctttcaattcaccatctgcatagtcagaggcgtgccataagcacgatttatcattcccagcgtgctcctgcaccgtcattgtcggtaacactagatgattggcgcagatcttgagagtcttagattgcctcataacaaggcgaacttttcctgtctctttgtgcttcaacagcttcacagtaccagcacctctctctttccagtgattcccttctttatcaaatcgatacagcttcgacttcagatcgaggatcggatcttcattttcctcgcctgtactgacagcgacttcctctaacttgacaataggggcgacttgagctccagtgtcttcgtcctcggcggcaggagcttcttcatcttctctgtgctcgtgttcgggttctttgcttgccat

SEQ ID NO: 26 - DNA, 672 bp, Gossypium hirsutum
  misc_feature (597)..(597): n is a, c, g, or t
cctataccccaaaagtatattctaccgccagcaaacctccaacgcctcactccaaacaaagcaatcatggcaagcaaagaaccggatcaggagcacagagaagacgaggatgccgcccccgccgaggaagaagacaccggagctcaaatcgcccctatcatcaagctcgaggaagtcgccgtcagcaccggcgaggaaaacgaagatccgatactcgatctgaagtcgaagctctaccgatttgataaagaaggcaatcaatggaaagagagaggcgctggtactgtgaagctgttgaagcacaaagagactggaaaagttcgccttgttatgaggcaatctaagactctcaagatctgcgccaatcatttagtgttgccgactatgacggtgcaggagcacgcagggaacgacaaatcgtgcctgtggcacgcttctgactacgctgatggtgaattgaaagatgatctgttctgcattcgttttgcatctgtgaaaaattgcaaaaccttcatgcaaaagttccaagaagttgctgaatcacaaaaaccgaaagaggaaaataaagatgcatctgctgctgctggactgctgganaatttgaccttgatnannaaaaaaccgagattaacctgattgaccttgatnannaaaaaaccgagattaacctga

SEQ ID NO: 27 - DNA, 672 bp, Gossypium hirsutum
  misc_feature (22)..(23): n is a, c, g, or t
tcaggttaatctcggttttttnntnatcaaggtcaaattntccagcagtccagcagcagcagatgcatctttattttcctctttcggtttttgtgattcagcaacttcttggaacttttgcatgaaggttttgcaatttttcacagatgcaaaacgaatgcagaacagatcatctttcaattcaccatcagcgtagtcagaagcgtgccacaggcacgatttgtcgttccctgcgtgctcctgcaccgtcatagtcggcaacactaaatgattggcgcagatcttgagagtcttagattgcctcataacaaggcgaacttttccagtctctttgtgcttcaacagcttcacagtaccagcgcctctctctttccattgattgccttctttatcaaatcggtagagcttcgacttcagatcgagtatcggatcttcgttttcctcgccggtgctgacggcgacttcctcgagcttgatgataggggcgatttgagctccggtgtcttcttcctcggcgggggcggcatcctcgtcttctctgtgctcctgatccggttctttgcttgccatgattgctttgtttggagtgaggcgttggaggtttgctggcggtagaatatacttttggggtataggaactggaactantnnttttttggctctaattggact

SEQ ID NO: 28 - DNA, 672 bp, Gossypium hirsutum
atggcaagcaaagagcccgatcacgagcacagagaagacgatgacgcccccgccgccgatgatgaagacaccggtgctcaagttgcccctatcgtcaggcttgaggaagtcgccgttaccaccggcgaggaagaagaagatgccatactcgatctgaagtcgaaactgtaccgatttgataaagacgggaatcaatggaaagagagaggcgctggtactgtgaagctgttgaagcacaaaaagaccggaaaagttcgccttgttatgaggcaatctaagactctcaagatctgcgccaatcattatgtgttggcaacgatgacggtgcaggagcacgcagggaacgacaaatcgtgcctatggcacgcttctgactttgctgatgatgaactgaaagatgaacttttctgcattcgttttgcatcagtggaaaattgcaaaagcttcatggaaacggtccgagaagttgctgaatcacaaaaatcaacagaggaaaataaagaggcatctgctgcagctggtctacttgagaagttgagtgttgaggaaaaggaagccaaggataaagtggaagagaaggatgttaaagcaaaggaagaaacggaaacaaaggaggatacaaaaaaagtagatgatagtgagaagaaagatggggagccagaatcaacttaa

SEQ ID NO: 29 - DNA, 672 bp, Gossypium hirsutum
ttaagttgattctggctccccatctttcttctcactatcatctactttttttgtatcctcctttgtttccgtttcttcctttgctttaacatccttctcttccactttatccttggcttccttttcctcaacactcaacttctcaagtagaccagctgcagcagatgcctctttattttcctctgttgatttttgtgattcagcaacttctcggaccgtttccatgaagcttttgcaattttccactgatgcaaaacgaatgcagaaaagttcatctttcagttcatcatcagcaaagtcagaagcgtgccataggcacgatttgtcgttccctgcgtgctcctgcaccgtcatcgttgccaacacataatgattggcgcagatcttgagagtcttagattgcctcataacaaggcgaacttttccggtctttttgtgcttcaacagcttcacagtaccagcgcctctctctttccattgattcccgtctttatcaaatcggtacagtttcgacttcagatcgagtatggcatcttcttcttcctcgccggtggtaacggcgacttcctcaagcctgacgataggggcaacttgagcaccggtgtcttcatcatcggcggcgggggcgtcatcgtcttctctgtgctcgtgatcgggctctttgcttgccat

SEQ ID NO: 30 - DNA, 684 bp, Gossypium hirsutum
atggcgaccgatgctaaagcagatccagagccgcactgcagagaagacgatgacaacgctcccgccaccgccgcagacgatgaggacaccggtgctcaagtagctcctatcgtcaagctcgaggaagtcgctgtcactactggtgaagaagacgaggatccgattctcgatctgaaggcaaagctgtatagattcgataaggaagggaatcaatggaaggagagaggagttggaaatgtgaagctcttgaagcacaaagtcaatggcaaagtccgtctagtcatgcgccaatctaaaacccttaagatctgtgctaaccatttcgttattccaacaatgacggtgcaagaacattctggaaatgataaatcatgcgtttggcacgccgctgatttcgctgatgggaatttgaaagaagagatgttctgtatcagatttgcttctcttgagaattgtaagaccttcatggagacagttcaagatgttgctgaaagtcagggaaagaaagaggaaaataaagacgccacagtcactgctgatctcctcgacaagttgagtgttggagaaagtaaaagtgacggtaaagaagaagatacggtcgttgcctccgacaccaaggaagccgtcaaagatgaagcaaaaacagaggaagatgagaaggatgaggctgcttcaaaagcatag

SEQ ID NO: 31 - DNA, 684 bp, Gossypium hirsutum
ctatgcttttgaagcagcctcatccttctcatcttcctctgtttttgcttcatctttgacggcttccttggtgtcggaggcaacgaccgtatcttcttctttaccgtcacttttactttctccaacactcaacttgtcgaggagatcagcagtgactgtggcgtctttattttcctctttctttccctgactttcagcaacatcttgaactgtctccatgaaggtcttacaattctcaagagaagcaaatctgatacagaacatctcttctttcaaattcccatcagcgaaatcagcggcgtgccaaacgcatgatttatcatttccagaatgttcttgcaccgtcattgttggaataacgaaatggttagcacagatcttaagggttttagattggcgcatgactagacggactttgccattgactttgtgcttcaagagcttcacatttccaactcctctctccttccattgattcccttccttatcgaatctatacagctttgccttcagatcgagaatcggatcctcgtcttcttcaccagtagtgacagcgacttcctcgagcttgacgataggagctacttgagcaccggtgtcctcatcgtctgcggcggtggcgggagcgttgtcatcgtcttctctgcagtgcggctctggatctgctttagcatcggtcgccat

SEQ ID NO: 32 - DNA, 723 bp, Zea mays
gccacgcgcagcagcaacggagcaggagcgcagccagcaagctcaggcccgagccctactgcaaccgccgctgctaccgcactgaacaccatggccgacccggcggagcaccgtccagcggaggaggacgaggaagccgcggcggccggcgaggatgaggacaccggcgctcaggtcgcgcccatcgtgaagctggaggaggtcgccgttaccaccggagaggaggacgaggacgtacttgtcgacatgaaggcgaagctctaccggttcgacaaggaggcgaaccagtggaaagaacgtggcacaggcactgtgaagctgcttaagcacaaggagaccgccaaggtccgcctcgtcatgcgtcaggcgaagacgcttaagatctgcgccaaccatctcgtggtggcgacgactaagatgcaggagcacgcggggagcgacaagtcgtgcgtgtggcacgcgctggacttcgccgacggcgagctcaaagaggagatgtttgctatccgttttggatctgtcgagaattgcaagaagttcaaggacactgttgaagagatagctgaacagcaaggaaagagcgaggagaaagaaaatgatgaagtctccactgccgcttctgatttggcacagaagttaacagtggcagagcccagcaaggaggaaaatgcagagaaagaggaggctcctgcatccgatgataagaaggatgataaagagtga

SEQ ID NO: 33 - DNA, 723 bp, Zea mays
tcactctttatcatccttcttatcatcggatgcaggagcctcctctttctctgcattttcctccttgctgggctctgccactgttaacttctgtgccaaatcagaagcggcagtggagacttcatcattttctttctcctcgctctttccttgctgttcagctatctcttcaacagtgtccttgaacttcttgcaattctcgacagatccaaaacggatagcaaacatctcctctttgagctcgccgtcggcgaagtccagcgcgtgccacacgcacgacttgtcgctccccgcgtgctcctgcatcttagtcgtcgccaccacgagatggttggcgcagatcttaagcgtcttcgcctgacgcatgacgaggcggaccttggcggtctccttgtgcttaagcagcttcacagtgcctgtgccacgttctttccactggttcgcctccttgtcgaaccggtagagcttcgccttcatgtcgacaagtacgtcctcgtcctcctctccggtggtaacggcgacctcctccagcttcacgatgggcgcgacctgagcgccggtgtcctcatcctcgccggccgccgcggcttcctcgtcctcctccgctggacggtgctccgccgggtcggccatggtgttcagtgcggtagcagcggcggttgcagtagggctcgggcctgagcttgctggctgcgctcctgctccgttgctgctgcgcgtggc

SEQ ID NO: 34 - DNA, 363 bp, Zea mays
gaacggggcacgggcaccgtcaagcttctcaagaacaaggagaccggcaaggtccgactggtcatgcgccaggccaagacgcttaagatctgcgcgaaccacctcgtggcccccaccacgagaatgcaggaacatgccggcagcgacaaatcgtgcgtctggcacgcttctgactttgcggatggcgaactcaaggaggagatgtttgccatcaggtttggttcggtagaaaactgcaagaagttcaaggagttggttgaggagattgctgagtcacttacagagaacgaggacaaggaaggtcaagatggctcatccacggccggattgctggagaagctcactgtgagcgagggcaaatct

SEQ ID NO: 35 - DNA, 363 bp, Zea mays
agatttgccctcgctcacagtgagcttctccagcaatccggccgtggatgagccatcttgaccttccttgtcctcgttctctgtaagtgactcagcaatctcctcaaccaactccttgaacttcttgcagttttctaccgaaccaaacctgatggcaaacatctcctccttgagttcgccatccgcaaagtcagaagcgtgccagacgcacgatttgtcgctgccggcatgttcctgcattctcgtggtgggggccacgaggtggttcgcgcagatcttaagcgtcttggcctggcgcatgaccagtcggaccttgccggtctccttgttcttgagaagcttgacggtgcccgtgccccgttc

SEQ ID NO: 36 - DNA, 1084 bp, Nicotiana tabacum
ggcacgaggagctgctccactgtttttactctctacaatcctctctgcatcacatccaatatccaatatcaatggcaagcactgacggtaaactcgagaacagggaagaggaagaagaagcagcccggagccgatgatgaagatacccgagctcaggttgcccaattgtcaagcttgaggaagttactgtttcaactggcgaggagaatgaagatgccatcgtcgatttgaaagccaagctttataggttcgataaagatgggaatcagtggaaagagagaggtgctggaactgtgaagctattgaagcataaggaaactggcaaagttcgccttgttatgcgccaatccaagacccttaagatctgtgccaaccaccttgttctcccaacgatgacgcttcaggaacatgctggaaatgagaagtcttgcttgtggcatgcaccagattttgctgatggtgaactgaaagatgagcttttctgcctcagatttgcatctatcgagaattgcaagtctttcatggagatgtttcaagaggttgctgaatcacaaaagaaagtggaaaatgaagatggatctacagctgctggactgcttgagaaattaagtgttgaagataaaaaggctgaagagaaaagcgaagctaaagccgaagacaaaaccaaggaaccccatggtaaggatgagaatccatccaaggatacagagaaaaaggatgaaagtgcttcagcctgaggggtatgcaccccgtgcactcgcccttgtagttttgaggttcatgatgccattccacaattccactttgaggttaagtggaattgtctttgttttttcttgaggttgggttggggttatgggaactgggaattggttggctttattagtctatcattgaggcgtgtgaacgtttacagaagttgtctcacttttaaggtcacgttctgtttctggttgaaatatttgtcgagtgggtgtaacaataggttttgtttgtattgtttgcgcacacaatgtacttcgccatttagtaatgcgtctgagttttgagtcccatttttacttgaaaaaaaaaaaaaaaaaaa

SEQ ID NO: 37 - DNA, 1084 bp, Nicotiana tabacum
tttttttttttttttttttcaagtaaaaatgggactcaaaactcagacgcattactaaatggcgaagtacattgtgtgcgcaaacaatacaaacaaaacctattgttacacccactcgacaaatatttcaaccagaaacagaacgtgaccttaaaagtgagacaacttctgtaaacgttcacacgcctcaatgatagactaataaagccaaccaattcccagttcccataaccccaacccaacctcaagaaaaaacaaagacaattccacttaacctcaaagtggaattgtggaatggcatcatgaacctcaaaactacaagggcgagtgcacggggtgcatacccctcaggctgaagcactttcatcctttttctctgtatccttggatggattctcatccttaccatggggttccttggttttgtcttcggctttagcttcgcttttctcttcagcctttttatcttcaacacttaatttctcaagcagtccagcagctgtagatccatcttcattttccactttcttttgtgattcagcaacctcttgaaacatctccatgaaagacttgcaattctcgatagatgcaaatctgaggcagaaaagctcatctttcagttcaccatcagcaaaatctggtgcatgccacaagcaagacttctcatttccagcatgttcctgaagcgtcatcgttgggagaacaaggtggttggcacagatcttaagggtcttggattggcgcataacaaggcgaactttgccagtttccttatgcttcaatagcttcacagttccagcacctctctctttccactgattcccatctttatcgaacctataaagcttggctttcaaatcgacgatggcatcttcattctcctcgccagttgaaacagtaacttcctcaagcttgacaattgggcaacctgagctcgggtatcttcatcatcggctccgggctgcttcttcttcctcttccctgttctcgagtttaccgtcagtgcttgccattgatattggatattggatgtgatgcagagaggattgtagagagtaaaaacagtggagcagctcctcgtgcc

SEQ ID NO: 38 - DNA, 756 bp, Solanum tuberosum
atggcactaatttcgaggcacacgacggcggcgttgtttgcgcctctgacgcaattatcgccgccgtcaacgccatcgaaattaaaacaaacatttttgaagaaaacatccttacttttttttttctttttcactaagcacgagcaccgggaagaggaagaagaagctgccggagccgatgatgaagataccggagctcaggtggctccaattgtcagactcgaagaagttgctgtatcaactggcgaggaaaatgaagatcccctcattgatttgaaagccaagctttataggtttgataaagatggtaatcagtggaaagagagaggtgctggaactgtgaagttattgaagcataaggaaactggcaaagttcgccttgttatgcgccaatccaaaacccttaagatctgcgccaaccactttgttctcccaacgatgacggttcaggaacatgctggaaatgagaagtcctgcgtgtggcatgcaccagattttgctgatggtgaactgaaagatgagcttttttgcatcagatttgcatctattgagaattgcaaatccttcatggatatgtttcaagaggtagctgaatcacaaaagaaagaggaaagtgaagacggatctactgctgccggactgcttgaaaagttaagtgttgaagataaaagccaggagaaagccaaatacaaaaccgagaaaggggatggtaaggaggagaaagctgaagacaaggacgcagaggccaccgcgtga

SEQ ID NO: 39 - DNA, 756 bp, Solanum tuberosum
tcacgcggtggcctctgcgtccttgtcttcagctttctcctccttaccatcccctttctcggttttgtatttggctttctcctggcttttatcttcaacacttaacttttcaagcagtccggcagcagtagatccgtcttcactttcctctttcttttgtgattcagctacctcttgaaacatatccatgaaggatttgcaattctcaatagatgcaaatctgatgcaaaaaagctcatctttcagttcaccatcagcaaaatctggtgcatgccacacgcaggacttctcatttccagcatgttcctgaaccgtcatcgttgggagaacaaagtggttggcgcagatcttaagggttttggattggcgcataacaaggcgaactttgccagtttccttatgcttcaataacttcacagttccagcacctctctctttccactgattaccatctttatcaaacctataaagcttggctttcaaatcaatgaggggatcttcattttcctcgccagttgatacagcaacttcttcgagtctgacaattggagccacctgagctccggtatcttcatcatcggctccggcagcttcttcttcctcttcccggtgctcgtgcttagtgaaaaagaaaaaaaaaagtaaggatgttttcttcaaaaatgtttgttttaatttcgatggcgttgacggcggcgataattgcgtcagaggcgcaaacaacgccgccgtcgtgtgcctcgaaattagtgccat

SEQ ID NO: 40 - DNA, 525 bp, Solanum tuberosum
gataaaacctctgttataagaaacacagcaaccgtacggttaacaatgtctagcgccgagcctaaacacgagcacagagaagaggaagaagccgctggagccgatgacgaagataccggagctcacgttgcgccgattgtcaggctcgaggaagttgccgtaacaactggcgaagagaacgaagatgctatcctcgatttgaaagcgaagctatatcgatttgacaaagagggaaatcagtggaaagagagaggtgctggtactgttaagcttttaaagcataaggaaactggaaaagttcgtcttgttatgcgtcaatctaagactcttaagatctgtgccaatcatctagttattccgacgatgacagttcaggaacatgctggaaacgaaaagtcctgtgtgtggcatgccgcagattttgctgatggtgaattgaaggatgaatttttctgtattagatttgcatcgttagagaattgcaagaccttcatggagactttccaagaggttgctgaatcgcaa

SEQ ID NO: 41 - DNA, 525 bp, Solanum tuberosum
ttgcgattcagcaacctcttggaaagtctccatgaaggtcttgcaattctctaacgatgcaaatctaatacagaaaaattcatccttcaattcaccatcagcaaaatctgcggcatgccacacacaggacttttcgtttccagcatgttcctgaactgtcatcgtcggaataactagatgattggcacagatcttaagagtcttagattgacgcataacaagacgaacttttccagtttccttatgctttaaaagcttaacagtaccagcacctctctctttccactgatttccctctttgtcaaatcgatatagcttcgctttcaaatcgaggatagcatcttcgttctcttcgccagttgttacggcaacttcctcgagcctgacaatcggcgcaacgtgagctccggtatcttcgtcatcggctccagcggcttcttcctcttctctgtgctcgtgtttaggctcggcgctagacattgttaaccgtacggttgctgtgtttcttataacagaggttttatc

SEQ ID NO: 42 - DNA, 687 bp, Triticum aestivum
  misc_feature (5)..(5): n is a, c, g, or t
caggnaattcggcacgaggctctggtacagcaagcgagcgccgccccaagccctacaccagcaatggccgatccagcggagcaccgcgaggaggaggaggaggccgcggcggccggcgaggaagaggacaccggcgcccaaatcgctcccatcgtgaagctcgaggaggtggccatcaccaccggcgaagaggacgaggacgtcctcctcgacatgaaagcgaagctctaccgatttgacaaggacgggggccagtggaaggagaggggcacgggtgccgtcaagctgctcaagcacaaggagactgccaaggtccgcctcgtcatgcgacaggccaagaccctcaagatctgcgccaatcacctcgttgtggccacgacgaagatgcaggagcacgctgggagcgacaagtcgtgcgtgtggcacgctctggattttgccgactgtgagctcaaggaggagatgttcgccatccgctttggatccgtggagaactgcaaaaaattcaaggatcttgtagatgagattgctgagcagcaaggaaagaatgaggagaaagaaagcgaggaagtctcctctgctgccgagttggtagagaaattgactgtggcggaggtcaaaaaggaggaacaaacggagaaagtggagactcctgcagtagatgataagaaggatgctgaaaagtga

SEQ ID NO: 43 - DNA, 687 bp, Triticum aestivum
  misc_feature (5)..(5): n is a, c, g, or t
gtccnttaagccgtgctccgagaccatgtcgttcgctcgcggcggggttcgggatgtggtcgttaccggctaggtcgcctcgtggcgctcctcctcctcctccggcgccgccggccgctccttctcctgtggccgcgggtttagcgagggtagcacttcgagctcctccaccggtagtggtggccgcttctcctgctcctgcaggaggagctgtactttcgcttcgagatggctaaactgttcctgcccccggtcaccttcctctccccgtgcccacggcagttcgacgagttcgtgttcctctgacggttccaggcggagcagtacgctgtccggttctgggagttctagacgcggttagtggagcaacaccggtgctgcttctacgtcctcgtgcgaccctcgctgttcagcacgcacaccgtgcgagacctaaaacggctgacactcgagttcctcctctacaagcggtaggcgaaacctaggcacctcttgacgttttttaagttcctagaacatctactctaacgactcgtcgttcctttcttactcctctttctttcgctccttcagaggagacgacggctcaaccatctctttaactgacaccgcctccagtttttcctccttgtttgcctctttcacctctgaggacgtcatctactattcttcctacgacttttcact

SEQ ID NO: 44 - DNA, 669 bp, Triticum aestivum
atggcggacaaggagcccgtcgtggagcgccccgtcgcggaggaggaaggcgaggactccgccgccgcggccgccgccggcgaggaggaggacaccggtgcccaggtcgcccccatcgtgcggctcgaggaggtcgccgtcaccaccggcgaggaggacgaggactctctcctcgacatgaaggcgaagctatacaggttcgacaaggatgggaaccagtggaaggagaggggcacgggcaccgtcaagctgctcaagcataaggagaacggcaaggtccgccttgttatgcgccaggccaagacacttaagatctgcgccaaccacctagtgatctcgaccacgaagatgcaggaacacgccggcagcgacaagtcctgtgtctggcacgcggcagactttgctgacggagagcttaaggatgagatgtttgccatccggtttggcaccgttgagaattgcaagaagtttaaagacttagttgatgagatcgctgagtcgcttgcaaagaatgaaggcggtgaaagcgaagaaggttcttccgcagctggactgctggagaaacttagtgtaagcgaaagcaaacctgaggaaagcgcagctaaggaggaatcaactgatgctagcaaggagacagaaaccaaagccgcgacagccccctcagagtag

SEQ ID NO: 45 - DNA, 669 bp, Triticum aestivum
ctactctgagggggctgtcgcggctttggtttctgtctccttgctagcatcagttgattcctccttagctgcgctttcctcaggtttgctttcgcttacactaagtttctccagcagtccagctgcggaagaaccttcttcgctttcaccgccttcattctttgcaagcgactcagcgatctcatcaactaagtctttaaacttcttgcaattctcaacggtgccaaaccggatggcaaacatctcatccttaagctctccgtcagcaaagtctgccgcgtgccagacacaggacttgtcgctgccggcgtgttcctgcatcttcgtggtcgagatcactaggtggttggcgcagatcttaagtgtcttggcctggcgcataacaaggcggaccttgccgttctccttatgcttgagcagcttgacggtgcccgtgcccctctccttccactggttcccatccttgtcgaacctgtatagcttcgccttcatgtcgaggagagagtcctcgtcctcctcgccggtggtgacggcgacctcctcgagccgcacgatgggggcgacctgggcaccggtgtcctcctcctcgccggcggcggccgcggcggcggagtcctcgccttcctcctccgcgacggggcgctccacgacgggctccttgtccgccat

SEQ ID NO: 46 - DNA, 666 bp, Medicago truncatula
atggccgatcccgagcaccgcgaagaggaggaagcacccgccgtcggtgacgacgaagacaccggagcacaagtcgctccgattgtccaactccaagaagttgccgtcactaccggtgaagaagacgaagaatccattctagatctcaaatcaaagctttaccgtttcgataaagatggaaatcagtggaaggagagaggtgctgggacggtcaagttcttgaagcataaggttactggtaaagtgaggcttctcatgaggcaatcgaagacactcaagatctgcgctaatcatctcattatacctacgatgagcgttcaagagcatgctgggaatgagaaatcttgtgtttggcatgctagggattttgctgatggtgaacttaaggatgagcttttctgcattcgatttccttcaattgaaaattgcaaaagctttatggaaacattccaagaagttgccgagtcccagaagcaagtggatgaccaggatgcatctgcagcagctgttcttgttgagaaattgactgttgaagacaaggctgctgatgcagagaagaaagatgaagagaagtccaaggataaaactggagagaaagaatctgcatcagaggcaagtaaggcagatgcagataaaaaagttgaagagcctgcatcctctgcttga

SEQ ID NO: 47 - DNA, 666 bp, Medicago truncatula
tcaagcagaggatgcaggctcttcaacttttttatctgcatctgccttacttgcctctgatgcagattctttctctccagttttatccttggacttctcttcatctttcttctctgcatcagcagccttgtcttcaacagtcaatttctcaacaagaacagctgctgcagatgcatcctggtcatccacttgcttctgggactcggcaacttcttggaatgtttccataaagcttttgcaattttcaattgaaggaaatcgaatgcagaaaagctcatccttaagttcaccatcagcaaaatccctagcatgccaaacacaagatttctcattcccagcatgctcttgaacgctcatcgtaggtataatgagatgattagcgcagatcttgagtgtcttcgattgcctcatgagaagcctcactttaccagtaaccttatgcttcaagaacttgaccgtcccagcacctctctccttccactgatttccatctttatcgaaacggtaaagctttgatttgagatctagaatggattcttcgtcttcttcaccggtagtgacggcaacttcttggagttggacaatcggagcgacttgtgctccggtgtcttcgtcgtcaccgacggcgggtgcttcctcctcttcgcggtgctcgggatcggccat

SEQ ID NO: 48 - DNA, 540 bp, Medicago truncatula
aaccctttatcactttctctctctaaaaaaatgtctacaagcaccgatcatcacgaagaggaagatgttcccgccggcgatgaagaagacaccggcgctcaaatcgctccgatcatccaacttcatgaagtcgctgtttctactggtgaagaagatgaagaagctattctcgacctaaaagcgaaactgtaccgatttgacaaggtagggaatcaatggaaggaacgaggggcaggaactgttaagtttttgaagcataaggttactggaaaagttaggcttgttatgagacaatcaaagactcttaagatctgtgctaatcatctcattttgcctaaaatgactgtgcaagagcatgctgggaatgagaaatcttgtgtttggcacgcgaaggactttgctgatggtgaattgaaagacgagtttttctgcattcgatttgcgtcaattgaaaattgcagaaagttcatagagacatttcaagaaattgctgaatccctgaacaaaagaggaaagcaaggatgcatccactgctgctga

SEQ ID NO: 49 - DNA, 540 bp, Medicago truncatula
tcagcagcagtggatgcatccttgctttcctcttttgttcagggattcagcaatttcttgaaatgtctctatgaactttctgcaattttcaattgacgcaaatcgaatgcagaaaaactcgtctttcaattcaccatcagcaaagtccttcgcgtgccaaacacaagatttctcattcccagcatgctcttgcacagtcattttaggcaaaatgagatgattagcacagatcttaagagtctttgattgtctcataacaagcctaacttttccagtaaccttatgcttcaaaaacttaacagttcctgcccctcgttccttccattgattccctaccttgtcaaatcggtacagtttcgcttttaggtcgagaatagcttcttcatcttcttcaccagtagaaacagcgacttcatgaagttggatgatcggagcgatttgagcgccggtgtcttcttcatcgccggcgggaacatcttcctcttcgtgatgatcggtgcttgtagacatttttttagagagagaaagtgataaagggtt

SEQ ID NO: 50 - DNA, 630 bp, Sorghum bicolor
atggccgaccccgcggagcaccgtccagcggaggaggaggaggaggccgcggcggccggcgaggatgaggacaccggcgcccaggtcgcccccatcgtgaagcttgaggaggtcgccgttaccaccggagaggaggacgaggacgtgctcctcgacatgaaagcgaagctataccggttcgataaggatgggaaccagtggaaagagcgcggaacaggcactgttaagctgctcaagcacaaggagaccgccaaggtccgcctcgtgatgcgtcaggcgaagacgcttaagatctgtgctaaccatctcgtggtggcgacgactaagatgcaggagcacgcggggagcgacaagtcgtgcgtgtggcacgcgctggacttcgccgaaggcgagctcaaggaggagatgttcgccatccgttttggatctgtcgagaattgcaagaagtttaaggatattgttgaagagatagctgaacagcaaggaaacactgaggagaaagaaaatgaggaagcctccactgccgctgatttggtagagaagttgacggtgacagaggccagcaaggaggaaactgtggagaaagaggaggctcctgcatctgatgataagaaggatgctaaagagtga

SEQ ID NO: 51 - DNA, 630 bp, Sorghum bicolor
tcactctttagcatccttcttatcatcagatgcaggagcctcctctttctccacagtttcctccttgctggcctctgtcaccgtcaacttctctaccaaatcagcggcagtggaggcttcctcattttctttctcctcagtgtttccttgctgttcagctatctcttcaacaatatccttaaacttcttgcaattctcgacagatccaaaacggatggcgaacatctcctccttgagctcgccttcggcgaagtccagcgcgtgccacacgcacgacttgtcgctccccgcgtgctcctgcatcttagtcgtcgccaccacgagatggttagcacagatcttaagcgtcttcgcctgacgcatcacgaggcggaccttggcggtctccttgtgcttgagcagcttaacagtgcctgttccgcgctctttccactggttcccatccttatcgaaccggtatagcttcgctttcatgtcgaggagcacgtcctcgtcctcctctccggtggtaacggcgacctcctcaagcttcacgatgggggcgacctgggcgccggtgtcctcatcctcgccggccgccgcggcctcctcctcctcctccgctggacggtgctccgcggggtcggccat

SEQ ID NO: 52 - DNA, 528 bp, Sorghum bicolor
gcacgaggggcgcagccagccctgctcccaaagcttctggagaacacctacccgtcttccctcccttgccccgccgtcgcgtgctcttcgaatcgaatcgcgccgctgtcgcctcacctcatggcggacaaggagcccgtcgtggagcgacccgaggcggctgaggaggaggacgcctcggccgccgccgccgccgcgggggaggaggaggacacgggcgcccaggtggcccccatcgtgcggctcgaggaggtagccgtcaccaccggcgaggaggacgaggacgtcctgctcgacatgaaggcgaagctgtaccgttttgacaaagacgggaaccaatggaaggaacgaggcacgggcaccgtcaagcttctcaagcacaaggagagcagcaaggtccgcctagtcatgcgccaggccaagacgcttaagatctgcgcgaaccacctcgtggcctccaccacgaaaatgcaggagcatgccggcagcgacaagtcgtgcgtctggcacgctgccgattttgcagat

SEQ ID NO: 53 - DNA, 528 bp, Sorghum bicolor
atctgcaaaatcggcagcgtgccagacgcacgacttgtcgctgccggcatgctcctgcattttcgtggtggaggccacgaggtggttcgcgcagatcttaagcgtcttggcctggcgcatgactaggcggaccttgctgctctccttgtgcttgagaagcttgacggtgcccgtgcctcgttccttccattggttcccgtctttgtcaaaacggtacagcttcgccttcatgtcgagcaggacgtcctcgtcctcctcgccggtggtgacggctacctcctcgagccgcacgatgggggccacctgggcgcccgtgtcctcctcctcccccgcggcggcggcggcggccgaggcgtcctcctcctcagccgcctcgggtcgctccacgacgggctccttgtccgccatgaggtgaggcgacagcggcgcgattcgattcgaagagcacgcgacggcggggcaagggagggaagacgggtaggtgttctccagaagctttgggagcagggctggctgcgcccctcgtgc

SEQ ID NO: 54 - DNA, 696 bp, Hordeum vulgare
cggcacgaggcgcaacaggtagcaacaactctggtagagcaagcgagcgccgccccaagccctactcccgcaatggccgatccagcggagcaccgcgaggaggaggaggaggccgcggcggcgggtgaggaagaggacaccggcgcccaaatcgctcccatcgtgaagcttgaggaggttgccatcaccaccggtgaagaggacgaggacgtcctcctcgacatgaaagcgaagctgtacagattcgacaaggacggggggcagtggaaggagcggggcacgggcaccgtcaagctgctcaagcacaaggagagttccaaggtccgcctcgtcatgcgacaggccaagaccctcaagatctgcgccaatcacctcgttgtggcgactacaaagatgcaggagcacgccgggagcgacaagtcgtgcgtgtggcacgcgctggattttgccgactgtgagctcaaggaggagatgttcgccatccgctttggatcggtggagaattgcaaaaaattcaaggatcttgtagacgagattgctgagcagcaaggaaagaatgaggagaaagaaagcgaggaggtttcgtctgctgccgagctggtagagaagttaagtgtaacagaggtcaaaaaggaggaacaaacggagaaagtggagactcctgcagtagatgataagaaggatgctgaagactga

SEQ ID NO: 55 - DNA, 696 bp, Hordeum vulgare
tcagtcttcagcatccttcttatcatctactgcaggagtctccactttctccgtttgttcctcctttttgacctctgttacacttaacttctctaccagctcggcagcagacgaaacctcctcgctttctttctcctcattctttccttgctgctcagcaatctcgtctacaagatccttgaattttttgcaattctccaccgatccaaagcggatggcgaacatctcctccttgagctcacagtcggcaaaatccagcgcgtgccacacgcacgacttgtcgctcccggcgtgctcctgcatctttgtagtcgccacaacgaggtgattggcgcagatcttgagggtcttggcctgtcgcatgacgaggcggaccttggaactctccttgtgcttgagcagcttgacggtgcccgtgccccgctccttccactgccccccgtccttgtcgaatctgtacagcttcgctttcatgtcgaggaggacgtcctcgtcctcttcaccggtggtgatggcaacctcctcaagcttcacgatgggagcgatttgggcgccggtgtcctcttcctcacccgccgccgcggcctcctcctcctcctcgcggtgctccgctggatcggccattgcgggagtagggcttggggcggcgctcgcttgctctaccagagttgttgctacctgttgcgcctcgtgccg

SEQ ID NO: 56 - DNA, 660 bp, Hordeum vulgare
atggcggacaaggagcccgtcgtggagcgccccgtcgcggaggaggaaggcgaggactccgcggccgccgccgccgccggcgaggaggaggacaccggtgcccaagtcgcccccatcgtgcggctcgaggaggtcgccgtcaccaccggtgaggaggacgaggactctctcctcgacatgaaggcgaagctatacaggttcgataaggacgggaaccagtggaaggagaggggcacaggcaccgttaagctgctcaagcacaaggagaacggcaaggtccgccttgttatgcgccaggccaagacgcttaagatctgcgccaaccacttagtgatctcgaccacgaagatgcaggaacatgccggcagcgacaaatcctgcgtctggcacgcggcagactttgctgacggagagctcaaggatgagatgtttgccattcggtttggcactgtagagaattgcaagaagtttaaggacttggttgatgagattgctgagtcgcttgcaaaggatgaagacggcgaaagcgtagaaggctcttctgcagctggactgctggagaagcttagcgtaaatgaaagcaaacctgaggaaagcgcagcaaaggaggaatcatccaaggagacagaaaccaaagccgcgacaaccccctcagagtag

SEQ ID NO: 57 - DNA, 660 bp, Hordeum vulgare
ctactctgagggggttgtcgcggctttggtttctgtctccttggatgattcctcctttgctgcgctttcctcaggtttgctttcatttacgctaagcttctccagcagtccagctgcagaagagccttctacgctttcgccgtcttcatcctttgcaagcgactcagcaatctcatcaaccaagtccttaaacttcttgcaattctctacagtgccaaaccgaatggcaaacatctcatccttgagctctccgtcagcaaagtctgccgcgtgccagacgcaggatttgtcgctgccggcatgttcctgcatcttcgtggtcgagatcactaagtggttggcgcagatcttaagcgtcttggcctggcgcataacaaggcggaccttgccgttctccttgtgcttgagcagcttaacggtgcctgtgcccctctccttccactggttcccgtccttatcgaacctgtatagcttcgccttcatgtcgaggagagagtcctcgtcctcctcaccggtggtgacggcgacctcctcgagccgcacgatgggggcgacttgggcaccggtgtcctcctcctcgccggcggcggcggcggccgcggagtcctcgccttcctcctccgcgacggggcgctccacgacgggctccttgtccgccat

SEQ ID NO: 58 - DNA, 654 bp, Lactuca sativa
gattcggccttatggcctgtagccggggacgaagacaccggagcgcaagttgctccgatcgtcaggcttgaagctgtcgaggtcattaacggtgaagaagacgaggatgcaatactcgatcttaagtcgaagttgtatagattcgacaaggacgggaatcagtggaaagaacgaggcgccggaaccgttaagcttctaaagcataaagaaactggaaaagttcgtcttgttatgcgtcaatcaaaaaccctaaagatctgcgccaatcatctcgttctaccgacgacatcggttcaagaacacgctggcaatgacaagtcatgcgtgtggcatgctgccgacttttctgatggtgaacttaaagacgaacttttctgcattagatttggatcagttgataattgcaaaaagttcatggaaacagttgaagaagtggcagagtcacagcatcccgagaaggaggagaacaaagatgcctcagatgctgctggactgctggagaatttgactgttaaagataagacagaagaagaaggaaaagaagtcaaagaggcatctgctgatgcaccaaagaaggaggagaaagagcctgaaaaggcggcagagtcagaggataagaagaaggaagaatcttcacctgcttcctcaacttaa

SEQ ID NO: 59 - DNA, 654 bp, Lactuca sativa
ttaagttgaggaagcaggtgaagattcttccttcttcttatcctctgactctgccgccttttcaggctctttctcctccttctttggtgcatcagcagatgcctctttgacttcttttccttcttcttctgtcttatctttaacagtcaaattctccagcagtccagcagcatctgaggcatctttgttctcctccttctcgggatgctgtgactctgccacttcttcaactgtttccatgaactttttgcaattatcaactgatccaaatctaatgcagaaaagttcgtctttaagttcaccatcagaaaagtcggcagcatgccacacgcatgacttgtcattgccagcgtgttcttgaaccgatgtcgtcggtagaacgagatgattggcgcagatctttagggtttttgattgacgcataacaagacgaacttttccagtttctttatgctttagaagcttaacggttccggcgcctcgttctttccactgattcccgtccttgtcgaatctatacaacttcgacttaagatcgagtattgcatcctcgtcttcttcaccgttaatgacctcgacagcttcaagcctgacgatcggagcaacttgcgctccggtgtcttcgtccccggctacaggccataaggccgaatc

SEQ ID NO: 60 - DNA, 883 bp, Oryza sativa
  misc_feature (8)..(8): n is a, c, g, or t
accgtgangnggaggaggaggccgcggcggcggcggtgcgangacnaggacaccggcgcccagntcgcgcccatcgtcaanctcgaggaggtcgccgtcaccaccggtgaggaggacgaggaggtcctcctcgacatgaagtcgaagctgtaccgcttcgacaaggaggggaaccaatggaaggagagangcaccggcacgtgaaagcttgctgaagcacaaggagaccggcaaggtccgtctcgtcatgcgccaggctaagacgctcaagatctgcgccaatcacctcgtggcgacgaccacgaagatgcaggagcacgccggcagcgacaagtcgtgcgtgtgcacgcgctggacttcgccgacggcgagctcaaggaggaaatgttccccatacgctttggatccgtcgagaactgcaagaagttcagggagatggtagaagagattgctgagcagcaaggaaagaacgaggagaaagaaaatgaggaagtctcctctactgcagggttggttgagaagctttcagtgaccgagacaaaaaaggaggaaaatgcagagaaagaggagactcctgcagaagaggataagaaggacgctaaagagtgaaagcacccgcaacctctaggcagctttgatatgcctgaagcgagtgaagtgttaaatgagcgtcatttcattcattttgtggtcaaagttcttcctttcacaaattttcattgtgttttcttttggatgaatgtttgtgctaggcaaatttgagttgtatcagtcgggttcttggttactacactgttacttaaaccttgagttgtttatccgagatggggtggccggagtgggggcacagcatgtttgtacaattatttgnngttgcttttgg

SEQ ID NO: 61 - DNA, 883 bp, Oryza sativa
  misc_feature (12)..(13): n is a, c, g, or t
ccaaaagcaacnncaaataattgtacaaacatgctgtgcccccactccggccaccccatctcggataaacaactcaaggtttaagtaacagtgtagtaaccaagaacccgactgatacaactcaaatttgcctagcacaaacattcatccaaaagaaaacacaatgaaaatttgtgaaaggaagaactttgaccacaaaatgaatgaaatgacgctcatttaacacttcactcgcttcaggcatatcaaagctgcctagaggttgcgggtgctttcactctttagcgtccttcttatcctcttctgcaggagtctcctctttctctgcattttcctccttttttgtctcggtcactgaaagcttctcaaccaaccctgcagtagaggagacttcctcattttctttctcctcgttctttccttgctgctcagcaatctcttctaccatctccctgaacttcttgcagttctcgacggatccaaagcgtatggggaacatttcctccttgagctcgccgtcggcgaagtccagcgcgtgcacacgcacgacttgtcgctgccggcgtgctcctgcatcttcgtggtcgtcgccacgaggtgattggcgcagatcttgagcgtcttagcctggcgcatgacgagacggaccttgccggtctccttgtgcttcagcaagctttcacgtgccggtgcntctctccttccattggttcccctccttgtcgaagcggtacagcttcgacttcatgtcgaggaggacctcctcgtcctcctcaccggtggtgacggcgacctcctcgagnttgacgatgggcgcganctgggcgccggtgtcctngtcntcgcaccgccgccgccgcggcctcctcctccncntcacggt

(19) United States
(12) Patent Application Publication
(10) Pub. No.: US 2004/0023395 A1
(43) Pub. Date: Feb. 5, 2004

(54) Transgenic tetraploid plants and methods of production

(76) Inventors: Soo-Hwan Kim, Taejeon-city (KR); Bin Goo Kang, Seoul (KR); Woo Sung Lee, Suwon-city (KR); Ho-II Kim, Suwon-city (KR); Hawk Bin-Kwon, Asan-city (KR)

Correspondence Address: RAE-VENTER LAW GROUP, P.C., P.O. BOX 1898, MONTEREY, CA, 93942-1898, US

(21) Appl. No.: 10/351,242

(22) Filed: Jan. 24, 2003

Related U.S. Application Data
(63) Continuation-in-part of application No. 10/181,202, filed on Nov. 18, 2002.
Application No. 10/181,202 is a 371 of international application No. PCT/KR01/01450, filed on Aug. 28, 2001.

(30) Foreign Application Priority Data
Aug. 28, 2000 (KR) 50067
Aug. 27, 2001 (KR) 51820

(51) Int. Cl. A01H 1/00; C12N 15/82

(52) U.S. Cl. 435/468; 800/286; 800/294

(57) ABSTRACT

The present invention relates to methods and compositions for the generation of high yield, super-productive transgenic plants that are disturbed in Ran/Ran-binding proteins-mediated cellular processes. Specifically, the present invention relates to a method for generating transgenic plants overexpressing in a sense or an antisense orientation Ran or various Ran-binding proteins to modify the biological processes in which those proteins are involved. These gene technologies provide ways to develop economically valuable super-productive crops the biomass and yields of which are increased 1.5-2.5 times or more those of currently available wild type crops. These increases in size and/or length can be seen in several organs of transgenic plants including leaf, stem, flower, roots, and seeds.

### CROSS-REFERENCE TO RELATED APPLICATIONS

This application is a continuation in part of U.S. Ser. No. 10/181,202 filed Jul. 10, 2002, which is a 35 USC 371 filing from PCT/KR01/01450 filed Aug. 28, 2001, which disclosures are hereby incorporated by reference.

### INTRODUCTION

1. Field of the Invention

The invention relates to compositions and methods for producing transgenic tetraploid plants. The method involves disturbing Ran/Ran-binding protein-mediated cellular processes by overexpression of sense or antisense orientation nucleic acid encoding Ran or Ran-binding proteins to modify biological processes in which these proteins are involved. The invention is exemplified by preparation of tetraploid Arabidopsis, tomato and tobacco plants having increased tissue biomass and/or seed yields and by production of tetraploid rice, potato and cotton.

2. Background

For centuries, humans have made improvements to crop plants through selective breeding and hybridization. Plant biotechnology is an extension of this traditional plant breeding with an important difference; it allows for the transfer of genetic information in a more precise, controlled manner. Traditional plant breeding can involve the movement of hundreds or thousands of genes, whereas plant biotechnology allows for the transfer of only one or a few desirable genes. This more precise science allows plant breeders to develop crops with specific beneficial traits and without undesirable traits.

A desirable trait in crop plants is high yield of the plant part or parts for which the plant is grown. For plants that are grown for other reasons, such as ornamentals and trees, it can be desirable for the entire plant to be of a larger phenotype than the parent plant. Chromosome doubling, called polyploidy, is a common natural phenomenon in several plant species that results in doubled genes. The process has occurred in many economically important crops, including soybeans, wheat, cotton, potatoes, alfalfa and sugar cane. These natural polyploids tend to have a larger phenotype and breeders have been selecting these out of the plant progeny pool for a long time. Often polyploid plants are not only larger but they are more robust than their ordinary counterparts and can have additional desirable characteristics, for example they can be larger flowered and be more intensely colored. See for example the following Plant Variety Protection Act patents: PP4,000; PP4221; PP10,388; and PP11,373.

Various methods have been developed by breeders to create polyploid cells. In plants, traditionally this is accomplished by application of colchicine which inhibits mitotic spindle formation which in turn leads to cells with various chromosome numbers. As an example, tetraploids can be produced by applying aqueous colchicine to diploid seedling apices. Often this treatment with colchicine results in death of the seedling or seed or produces aneuploids (cells with extra or missing chromosomes) or polyploids. Plant hormones such as kinetin, 6-benzylaminopurine, zeatin, and 2-isopentenyladenine also have been used. New varieties of plants that are tetraploid have been developed for example by fusing protoplasts from two different diploid plants and hybrids have been produced by crossing a first inbred line of a tetraploid plant with a non-identical second inbred line of the tetraploid plant to form a hybrid tetraploid seed. Transgenic methods also have been used, including the use of a cytotoxic gene that encodes a product that disrupts normal functioning of cells. Examples include the use of genes for the restriction enzyme EcoRI (Barnes and Rine, Proc. Natl. Acad. Sci. USA 82:1354-1358 (1985)), diphtheria toxin A (Yamaizumi et al., Cell 15:245-250 (1987)), streptavidin (Sano and Cantor, Proc. Natl. Acad. Sci. USA 87:142-146 (1990)), and barnase (Paddon and Hartley, Gene 53:11-19 (1987)). Expression of diphtheria toxin A resulted in transgenic plants without flowers (Nilsson et al., Plant Journal. 15:799-804 (1998)). Streptavidin is an anti-tumor molecule that is not naturally produced in plants. The streptavin expressed by transgenic plants is stored in the vacuole, and the transgenic plants are fertile but without an increase in yield (Murray et al., Transgenic Research 11:199-214 (2002)). Barnase A is an extracellular ribonuclease and used to induce male sterility in plants by causing cell lethality and ablation. Therefore, it cannot be used for the purpose of seed yield improvement (Burgess et al., Plant Journal, 31:113-125 (2002)).

Molecular farming as been used to produce compounds in plant cells as a way of avoiding potential diseases associated with using other eukaryotic host cells such as mammalian cells. However, industrial application of plant cell culture for production of plant derived pharmaceuticals and other compounds of interest has been limited by a number of drawbacks including slow growth rates, low product yields, intracellular storage of product, and poorly understood metabolic regulation. Perhaps the largest roadblock to industrial application of plant cell culture is low productivity per volume (Ten Hoopen, H., et al., In: Progress In Plant Cellular And Molecular Biology; Nijkamp et al. Eds; Kluwer Academic publishers: Boston, 673-681 (1990)). It therefore is of interest to develop methods and compositions for the preparation of fertile tetraploid plants having increased tissue biomass, particularly of agriculturally significant plant tissues and to develop cell cultures from tetraploid plants as a means of increasing yield of a product of interest.

Relevant Literature

The cDNA sequence and some biochemical characteristics of the AtRanBP1b gene, but not its biological functions, were described in Haizel et al The Plant Journal (1997) 11: 93-103. A cDNA of a pea Ran/TC4 (PsRan1), and sequences for AtRanBP1 c and AtRanBP1b are disclosed in Kim, S. -H. (1997) Dissertation, University of Texas at Austin. Antisense AtRanBP1c and PsRan1 transgenic Arabidopsis plants and constructs with these sequences in the binary vector pLBJ21, which contains a CaMV 35S promoter, are disclosed. Also disclosed is a sense AtRanBP1b construct in the binary vector pLBJ21.

Ran (Ras-related nuclear) is a unique small GTP-binding protein predominantly localized in the nuclei. It plays an important role, with the aid of a family of Ran-binding proteins (RanBPs), in the regulation of nuclear protein transport and cell cycle progression.

Modulation of GTP-or GDP-bound state of Ran is achieved by the action of interacting (binding) proteins, such as RCC1 (a Ran nuclear guanine nucleotide exchange factor), Ran-GTPase-activating protein (RanGAP), Ran-binding protein I (RanBP1, a RanGAP cofactor), and Mog1 (a guanine nucleotide release factor) (Kahana, J. A., and Cleveland, D. W., (1999) J. Cell Biol. 146,1205-1210). RCC1 is a Guanine nucleotide exchange factor (GEF) that exchanges Ran-bound GDP with GTP, Ran-GAP is a Ran-GTPase activation protein that catalyzes the GTPase activity of Ran, and RanBP1 is a cofactor that aids the activity of RanGAP by stabilizing a Ran-GTP state (Hopper, A. K. Traglia, H. M., and Dunst, R. W. (1990) J. Cell Biol, 111,309-321; Koepp, D. M., and Silver, P. A. (1996) Cell 87,1-4). Such RanBPs cause the unequal distribution of Ran-GTP and Ran GDP in cytoplasm and nucleoplasm in the prophase of cell division. Specifically, Ran-GTP becomes abundant on the nucleoplasmic side of a nuclear pore complex and Ran-GDP becomes predominant on the cytoplasmic side of the complex (Kahana, J. A., and Cleveland, D. W. (1999) J. Cell Biol. 146,1205-1210).

Mitotic cells replicate, increase the number of chromosomes 2-fold, and transmit them to daughter cells evenly. This mitotic process consists of prophase, metaphase, anaphase and telophase. Ran/RanBP signal transduction pathway in concert with RanBP1, RanBPM, RCC1 and RanGAP, etc. have been shown recently to play critical roles in such processes in experiments using animal cells and yeast as model organisms. RCC1 maintains the concentration of Ran-GTP in nuclei at a high level while RanBP I or RanGAP increases the level of Ran-GDP in the same nuclei. RanBPM promotes the formation of chromosomal microtubule spindles in a Ran-GTP dependent manner (Wilde, A., and Zheng, Y. (1999) Science 284,1359-1362). These spindles move the chromosomes to opposite poles, thereby promoting cell cycle progression and division (Nakamura, M., Masuda, H., Horri, J., Kuma, K. I., Yokoyama, N., Ohba, T., Nishitani, H., Miyata, T., Tanaka, M., and Nishimoto, T. (1998) J. Cell. Biol. 143,1041-1052).

The mitotic effects of a RanBP 1 in yeast have been reported in various studies (Battistoni, A., Guar guaglini, G., Degrassi, F., Pittoggi, C., Palena, A., Matteo, G. D., Pisano, C., Cundari, E., and Lavia, P. C. (1997) J. Cell Sci. 110, 2345-2357; He, X., Hayashi, N., Walcott, N. G., Azuma, Y., Patterson, T. E., Bischoff, F. R., Nishimoto, T., and Sazer, S. (1998) Genetics 148,645-656; Kalab, P., Pu, R. T., and Dasso M. (1999) Curr. Biol. 9,481-484). It also has been reported that overexpression of AtRanBP1c in yeast inhibits formation of normal septum (Xia et al., 1996, Paint J. 10 (4): 761-769); the nucleus is condensed and the arrangement of actin becomes irregular. In addition, Ouspenski reported that a RanBP1 mutant (yrb 1-21) caused defects in proper arrangement and the formation of mitotic spindles, and thus inhibits normal mitotic progression. (Ouspenski, 1.1. (1998) Exp. Cell Res. 244, 171-183).

Plant Ran has a similar function to that observed when the Ran is expressed in yeast. This implies that the biological functions of Ran/RanBP pathways in plants might be similar to those of yeast or mammalian cells. Ach and Gruissem showed that a tomato Ran protein is functionally homologous to a yeast Ran-like protein (Ach and Gruissem. (1994) Proc. Natl. Acad. Sci. USA 91,5863-5867). In addition, sequences of Ran1, Ran2 and Ran3 have been identified in Arabidopsis thaliana. Their expression pattern and biochemical properties also have been reported (see Haizel et al (1997) supra). Antisense expression of AtRanBP1c or expression of yeast RanBP1 homologs has been shown to arrest mitotic progression from metaphase to interphase in rapidly growing cells. However, it was not known that modification of Ran/RanBP signal transduction can cause an inheritable chromosome doubling in plants, resulting in increased biomass and seed yield.

### SUMMARY OF THE INVENTION

The invention relates to the use of DNA constructs having as components a transcriptional and translational initiation region functional in plant cells and a DNA sequence encoding a Ran protein or a Ran-binding protein which when expressed modifies one or more biological process in which the expressed protein is involved and thereby alters the genotype and phenotype of the plant and/or cells in tissues of the plant expressing the protein. The construct also has a transcriptional termination region as an additional component. At least one of the components is exogenous to at least one other of said components, i.e., the construct components do not naturally occur together as a group. The DNA sequence in the DNA construct can be in either the sense or the antisense orientation with respect to the initiation region. The initiation region can be constitutive or inducible. The method includes the step of transforming a cell from a plant of interest with the DNA construct, generating a plant from the transformed cell and growing it so that the DNA sequence is expressed. A plant that expresses the Ran protein or Ran binding protein or in which production of these proteins is inhibited, exhibits a polyploid genotype and altered phenotypic characteristics both of the plant as a whole and of individual tissues, including increased size, increased biomass, and increased number and size of seeds. The invention thus finds use for example in producing polyploid cells for in vitro high yield production of one or more compound of interest as well as in producing plants of a large phenotype, both of the overall plant itself and of individual tissues such as leaves, roots, stems, flowers and seeds. The invention also finds use in modifying characteristics of particular tissues, such as length and hairiness of the root.

It is an object of the present invention to provide a method for generating transgenic plants by molecular genetic technologies exhibiting superior properties, namely having high yield and super-productivity.

It is another object of the present invention to provide recombinant vectors that can be transformed into crops by the above method.

It is another object of the present invention to provide genes that can be transformed into crops by the above method.

In order to achieve these objects, the present invention provides a Ran base sequence, as shown in SEQ.ID.NO:1.

In addition, the present invention also provides an AtRanBP1b base sequence, as shown in SEQ.ID.NO:2.

In addition, the present invention also provides an AtRanBP1c base sequence, as shown in SEQ.ID.NO:3.

In addition, the present invention also provides an antisense AtRanBP1b base sequence, as shown in SEQ.ID.NO:4, which suppresses the expression of endogenous AtRanBP1b gene.

In addition, the present invention also provides an antisense AtRanBP1c, as shown in SEQ.ID.NO:5, which suppresses the expression of endogenous AtRanBP1c gene.

The present invention also provides a recombinant vector harboring the antisense AtRanBP1b base sequence shown in SEQ.ID.NO:4 or a part of the antisense AtRanBP1b base sequence.

The present invention also provides a recombinant vector harboring the antisense AtRanBP1c base sequence shown in SEQ.ID.NO:5 or a part of the antisense AtRanBP1c base sequence.

The present invention also provides a recombinant vector harboring a gene which is selected from a group of Ran and Ran-binding proteins consisting of Ran, AtRanBP1a, AtRanBP1b, AtRanBP1c, RanGAP, RanBPM, RCG1 and RanBP1.

The present invention also provides transgenic plants transformed with a recombinant vector harboring a sense or an antisense base sequence of genes involved in Ran-mediated cellular processes.

The present invention also provides a method for generating transgenic plants transformed with a recombinant vector which contains a sense or an antisense base sequence of genes involved in Ran-mediated cellular processes, or knocked out genes, by T-DNA or transposons.

### BRIEF DESCRIPTION OF THE DRAWINGS

FIG. 1 shows a schematic diagram for the construct of a pLBJ21/PsRan vector.

FIG. 2 shows a schematic diagram for the construct of a pLBJ21/AtRanBP1b vector.

FIG. 3 shows a schematic diagram for the construct of a pLBJ21/AtRanBP1c vector.

FIG. 4 shows a schematic diagram for the construct of a pLBJ21/antisense AtRanBP1b vector.

FIG. 5 shows a schematic diagram for the construct of a pLBJ21/antisense AtRanBP1c vector.

FIG. 6 shows a genomic southern blot analysis for transgenic Arabidopsis (pLBJ21/PsRan).

FIG. 7 shows a genomic southern blot analysis for transgenic Arabidopsis (pLBJ21/antisense AtRanBP1c).

FIG. 8 shows a northern blot analysis for transgenic Arabidopsis (pLBJ21/PsRan).

FIG. 9 shows a northern blot of for transgenic Arabidopsis (pLBJ21/antisense AtRanBP1c).

FIG. 10 compares the root length of transgenic Arabidopsis (pLBJ21/PsRan) of the present invention and that of wild type plants.

FIG. 11 compares the length of roots for transgenic Arabidopsis plants (pLBJ21/antisense AtRanBP1c) of the present invention and that of wild type plants.

FIG. 12 compares the size of seeds for a transgenic Arabidopsis (pLBJ21/antisense AtRanBP1b) of the present invention and that of wild type plants.

FIG. 13 compares the size of flower for a transgenic Arabidopsis (pLBJ21/antisense AtRanBP1b) of the present invention and that of wild type plants.

FIG. 14 compares the size of leaves for a transgenic Arabidopsis (pLBJ21/antisense AtRanBP1b) of the present invention and that of wild type plants.

FIG. 15 compares adult the size of an adult plant for a pLBJ21/antisense AtRanBP1b transgenic plant of the present invention and that of wild type plants.

FIG. 16 compares the size of an adult plant for a transgenic tomato (pLBJ21/AtRanBP1b) of the present invention and that of wild type plants.

FIG. 17 compares the size of leaf for a transgenic tomato (pLBJ21/AtRanBP1b) of the present invention and that of wild type plants.

FIG. 18 compares the size of branch for a transgenic tomato (pLBJ21/AtRanBP1b) of the present invention and that of wild type plants.

FIG. 19 compares the size of immature fruit for a transgenic tomato (pLBJ21/AtRanBP1b) of the present invention and that of wild type plants.

FIG. 20 shows an alignment of the amino acid sequences of Arabidopsis and soybean Ran binding proteins.

FIG. 21 shows an alignment of amino acid sequences of AtRanBP1 and GhRanBP1-1.

FIG. 22 shows an alignment of the amino acid sequences of AtRanBP1 and GhRanBP1-2.

FIG. 23 shows an alignment of amino acids sequences of AtRanBP1b homologs of Arabidopsis and cotton.

FIG. 24 shows an alignment of amino acid sequences of Arabidopsis and maize Ran binding proteins.

FIG. 25 shows an alignment of amino acid sequences of NtRanBP1 and AtRanBP1b.

FIG. 26 shows a comparison of the size of tobacco seeds from wild type and transgenic plants.

FIG. 27 shows a schematic diagram of constructs.

FIG. 28 shows alignment of amino acid sequences of Arabidopsis and potato Ran-binding proteins.

FIG. 29 shows an alignment of amino acid sequences of Arabidopsis and wheat Ran-binding proteins.

FIG. 30 shows an alignment of the amino acid sequences of Arabidopsis and alflalfa Ran-binding proteins.

FIG. 31 shows an alignment of the amino acid sequences of Arabidopsis and sorghum Ran-binding proteins.

FIG. 32 shows an alignment of the amino acid sequences of Arabidopsis and barley Ran-binding proteins.

FIG. 33 shows an alignment of the amino acid sequences of Arabidopsis and lettuce Ran-binding proteins.

FIG. 34 shows a comparison of the size of tobacco cells in suspension culture of wild type and transformants (Mixo and 4n Sense).

FIG. 35 shows a Giemsa dye stained cell of a transgenic Arabidopsis expressing antisense AtRanBP1b.

FIG. 36 shows the ploidy level of transgenic tobacco as determined by flow cytometry.

### DETAILED DESCRIPTION OF THE INVENTION

In accordance with the subject invention, a method is provided for influencing the size and productivity of a plant of interest by using DNA constructs containing a sense or an antisense DNA sequence encoding a Ran protein or a Ran-binding protein to transform the plant or a parent of the plant and growing the plant to express the DNA sequence. Also provided is a method whereby modification of the ploidy of the transgenic plant can be achieved. Preferably the ploidy is tetraploid. In addition, the present invention provides methods of generating transgenic plants with modified levels of Ran or Ran-binding proteins (RanBP) by overexpressing, inhibiting or knocking-out these genes. The plant of interest can be monocotyledenous, for example rice, maize, wheat, sorghum, barley and sugarcane, or dicotyledenous, for example cotton, soybean, sunflower, canola and tobacco. Other plants of interest include trees such as poplar, eucalyptus, loblolly pine, poplar and sweet gum, and root crops such as ginseng, grasses, and cassaya.

The Ran protein or Ran-binding protein when expressed in plants modifies one or more biological process in which the expressed protein is involved. Transgenic plants can be generated by the overexpression of Ran or RanBP, by the suppression of the endogenous genes by the expression of complementary antisense RNA, or by the suppression of endogenous genes by the expression of the corresponding double-stranded RNA (RNA interference) using various plant promoters or other promoters functional in plant cells. Plants can be also generated by knocking-out the genes using UV-treatment, EMS-treatment, or a transposon. The antisense method is a method for inhibiting the translation of an mRNA of target gene by expressing the complementary strand (antisense) to the target genes in the organism to produce a bond between mRNA of the target genes and antisense mRNA, thereby preventing the mRNA of the target genes from being translated into protein.

The present invention offers several advantages over existing technologies that have been used to develop polyploid plant cells and plants. Polyploid plants frequently are sterile. This means that they flower, but that flowers produce no seeds. Using the methods of the invention however, not only are the plants fertile but the size and/or number of seeds in the transgenic plants is increased over those in their non-transgenic counterparts. The polyploid plants are generally larger and more vigorous than their diploid counterparts. They also have a greater capacity to adapt to a broad range of environmental conditions. Furthermore, cells from polyploid plants often show an increase in both volume of the cells themselves and increased production of biological contents, such as secondary metabolites.

The invention also can be used with cultured cells. When used in a cell culture system, as compared with the whole plant system, additional advantages are realized. The plant cell culture system does not constrain environmental and geographic limits in order to produce plant-derived proteins, secondary metabolites such as biopharmaceuticals, and other cellular products. Use of a cell culture system can also solve problems of molecular farming associated with slow growth of the whole plant, low concentrations of cellular products in the whole plant, and environmental concerns related to collecting wild plants. Plant cells can more easily be cultured in vitro as compared with animal cells, and plant-derived products are cheap to produce and store, easy to scale up for mass production and safer than those derived from animals. Plant cell culture also offers several potential advantages over harvest and extraction of whole plants. Continuous production, rather than seasonal, is possible in a bioreactor where conditions can be controlled for optimal product formation. Plant cell culture can provide a high quality, uniform product while reducing labor costs and eliminating the effects of weather and disease. Suspended cells cultured from transgenic plants with doubled chromosome numbers are several fold larger in diameter than those from wild type plants. Cell culture systems employing genetically transformed tetraploid cells thus provide a powerful tool for obtaining an increased yield of plant-derived cellular products.

The nucleotide sequence that provides for modification of the genotype of the transgenic plant as well as phenotype of the transgenic plant itself and/or plant cells, tissues or parts of interest is an open reading frame encoding a Ran protein or a Ran-binding protein. The nucleotide sequences of this invention may be synthetic, naturally derived, or combinations thereof. Depending upon the nature and/or source of the nucleotide sequence, it may be desirable to synthesize the sequence with plant preferred codons. The plant preferred codons may be determined from the codons of highest frequency in the proteins expressed in the largest amount in the particular plant species of interest to be transformed. Phenotypic modification can be achieved by modulating production either of an endogenous transcription or translation product, for example as to the amount, relative distribution, or the like, or an exogenous transcription or translation product, for example to provide for a novel function or products in a transgenic host cell or tissue. The present invention thus provides methods for generating transgenic plants of super-productive, high-yielded crops by overexpression or suppression of sense or antisense sequences for a group of proteins that are involved in Ran-mediated cellular processes.

Nucleotide sequences encoding proteins involved in Ran-mediated cellular processes which find use in the present invention include those encoding Ran (Haizel et al., 1997, Plant J 11 (1) 193-103) and Ran-binding proteins. DNAs encoding encoding Ran and Ran-binding proteins can be identified in a variety of ways. In one method, a source of a desired gene encoding a Ran protein or a Ran-binding protein, for example a genomic or cDNA library from Arabidopsis or Pisum sativum is screened with detectable enzymatically- or chemically-synthesized probes. The probes can be made from DNA, RNA, or non-naturally occurring nucleotides, or mixtures thereof. Probes can be enzymatically synthesized from DNAs of known genes encoding Ran and Ran-binding proteins for normal or reduced-stringency hybridization methods. For discussions of nucleic acid probe design and annealing conditions, see, for example, Sambrook et al, Molecular Cloning: A Laboratory Manual (2.sup.nd ed.), Vols. 1-3, Cold Spring Harbor Laboratory, (1989) or Current Protocols in Molecular Biology, F. Ausubel et al, ed., Greene Publishing and Wiley-Interscience, New York (1987), each of which is incorporated herein by reference. BLAST searching of cDNA libraries of a plant of interest also can be used to identify homologs of the Ran and Ran-binding proteins. Techniques for manipulation of nucleic acids encoding Ran and Ran-binding proteins such as subcloning nucleic acid sequences encoding polypeptides into expression vectors, labelling probes, DNA hybridization, and the like are described generally in Sambrook, supra.

Oligonucleotide probes also can be used to screen sources and can be based on sequences of known genes encoding Ran and Ran-binding proteins, including sequences conserved among known genes encoding Ran and Ran-binding proteins, or on peptide sequences obtained from a desired purified protein. Oligonucleotide probes based on amino acid sequences can be degenerate to encompass the degeneracy of the genetic code, or can be biased in favor of the preferred codons of the source organism. Alternatively, a desired protein can be entirely sequenced and total synthesis of a DNA encoding that polypeptide performed.

Once the desired DNA has been isolated, it can be sequenced by known methods. It is recognized in the art that such methods are subject to errors, such that multiple sequencing of the same region is routine and is still expected to lead to measurable rates of mistakes in the resulting deduced sequence, particularly in regions having repeated domains, extensive secondary structure, or unusual base compositions, such as regions with high GC base content. When discrepancies arise, resequencing can be done and can employ special methods. Special methods can include altering sequencing conditions by using: different temperatures; different enzymes; proteins which alter the ability of oligonucleotides to form higher order structures; altered nucleotides such as ITP or methylated dGTP; different gel compositions, for example adding formamide; different primers or primers located at different distances from the problem region; or different templates such as single stranded DNAs. Sequencing of mRNA also can be employed.

For the most part, some or all of the coding sequences for the polypeptides having the activity of Ran and Ran-binding proteins are from a natural source. In some situations, however, it is desirable to modify all or a portion of the codons, for example, to enhance expression, by employing host preferred codons. Host preferred codons can be determined from the codons of highest frequency in the proteins expressed in the largest amount in a particular host species of interest. Thus, the coding sequence for a polypeptide having Ran and Ran-binding protein activity can be synthesized in whole or in part. All or portions of the DNA also can be synthesized to remove any destabilizing sequences or regions of secondary structure which would be present in the transcribed mRNA. All or portions of the DNA also can be synthesized to alter the base composition to one more preferable to the desired host cell. Methods for synthesizing sequences and bringing sequences together are well established in the literature. In vitro mutagenesis and selection, site-directed mutagenesis, or other means can be employed to obtain mutations of naturally occurring genes encoding Ran and Ran-binding proteins to produce a polypeptide having Ran and Ran-binding activity in vivo with more desirable physical and kinetic parameters for function in the host cell, such as a longer half-life.

Preferred Ran-binding proteins for use in the subject invention include AtRanBP1a (Haizel et al., 1997, Plant J 11 (1): 93-103), AtRanBP1b (Haizel et al., 1997, Plant J 11 (1): 93-103), AtRanBP1c (AT5 in Xia et al., 1996, Plant J. 10 (4): 761-769), RanGAP (Merril et al., 1999 Science, 283: 1742-1745), RanBPM (Nakamura et al., 1998, J. Cell. Biol., 143 (4): 1041-1052), RCC1 (Fruno et al., 1991, Genomic, 11 (2): 459-461), and RanBP1 (Ouspenski et al., 1995, J. Biol. Chem., 270 (5): 1975-1978). The AtRanBP1a, AtRanBP1b and AtRanBP1c are Ran-binding proteins found in Arabidopsis, and the RanGAP, RanBPM, RCC1 and RanBP1 are Ran-binding proteins found in animals or yeast, etc. Other Ran and Ran-binding proteins that have been isolated include those from soybean, cotton, corn, tobacco, potato, wheat, alfalfa, sorghum, barley, and lettuce (see Examples). The amino acid sequences of Ran proteins are well conserved among organisms; they are 90% or more identical to each other in plants, and are 70% or more identical to those found in yeast (Merkle et al., 1994, Plant J. 6 (4): 555-565). The DNA sequences of AtRanBP1 in plants are 80% or more identical to each other, and are 60% or more identical to those of other organisms (Haizel et al., 1997, Plant J. 11 (1): 93-103). On the other hand, the amino acid sequence of CST20, a RanBP1 of yeast, is approximately 50% identical to that of RanBP1 of mice (Ouspenski et al., 1995, JBC 270 (5): 1975-1978). The amino acid sequence of RanBPM of mice is almost identical to that of human, and approximately 30% identical to that of yeasts (Nakamura et al., 1998, J. Cell. Biol., 143 (4): 1041-149). The amino acid sequence of RanGAP of Drosophilae is 34 to 36% identical to those of RanGAP in yeasts and mice (Merril et al., 1999, Science, 12: 283-287). Thus, it is reasonable to say that Ran is a family of proteins that are 70% or more identical in their amino acid sequences, and AtRanBP1b or AtRanBP1c is a family of proteins that are 50% or more identical in their amino acid sequences. Accordingly, other RanBPs such as RanGAP, RCC1, and RanBPM form a group of proteins that are 35% or more identical in their amino acid sequences.

Other nucleotide sequences that can be used include those that encode Ran-binding domains. Many Ran-binding proteins have a stretch of amino acid sequences called a Ran-binding domain (RanBD) to which the Ran protein binds (Beddow et al., 1995, PNAS, 92: 3328-3332). This Ran-binding domain aids the hydrolysis of GTP by binding to Ran protein. (Novoa et al., 1999, Mol. Biol. Cell., 10: 2175-2190). Therefore, another group of proteins involved in the Ran-mediated cellular processes is one that includes Ran-binding domains. Other DNAs which are substantially identical in sequence to the Arabidopsis and Pisum sativum genes encoding Ran protein or Ran-binding protein, or which encode polypeptides which are substantially similar to these protein can be used. By substantially identical in sequence is intended an amino acid sequence or nucleic acid sequence exhibiting in order of increasing preference at least 60%, 80%, 90% or 95% homology to the DNA sequence of the Arabidopsis genes or nucleic acid sequences encoding the amino acid sequences for such genes. For polypeptides, the length of comparison for sequences generally is at least 16 amino acids, preferably at least 20 amino acids, and most preferably 35 amino acids. For nucleic acids, the length of comparison for sequences generally is at least 50 nucleotides, preferably at least 60 nucleotides, and more preferably at least 75 nucleotides, and most preferably, 110 nucleotides.

Homology typically is measured using sequence analysis software, for example, the Sequence Analysis software package of the Genetics Computer Group, University of Wisconsin Biotechnology Center, 1710 University Avenue, Madison, Wis. 53705, MEGAlign (DNAStar, Inc., 1228 S. Park St., Madison, Wis. 53715), and MacVector (Oxford Molecular Group, 2105 S. Bascom Avenue, Suite 200, Campbell, Calif. 95008). BLAST (National Center for Biotechnology Information (WCBI) www.ncbi.nlm.gov; FASTA (Pearson and Lipman, Science (1985) 227:1435-1446). Such software matches similar sequences by assigning degrees of homology to various substitutions, deletions, and other modifications. Conservative substitutions typically include substitutions within the following groups: glycine and alanine; valine, isoleucine and leucine; aspartic acid, glutamic acid, asparagine, and glutamine; serine and threonine; lysine and arginine; and phenylalanine and tyrosine. Substitutions may also be made on the basis of conserved hydrophobicity or hydrophilicity (Kyte and Doolittle, J. Mol. Biol. (1982) 157: 105-132), or on the basis of the ability to assume similar polypeptide secondary structure (Chou and Fasman, Adv. Enzymol. (1978) 47: 45-148, 1978). A related protein to the probing sequence is identified when p□ 10⁻⁷ to 10⁻⁸.

Once the DNA sequences encoding the Ran protein or Ran-binding protein have been obtained, they are placed in a vector capable of replication in a host cell, or propagated in vitro by means of techniques such as PCR or long PCR. Replicating vectors can include plasmids, phage, viruses, cosmids and the like. Desirable vectors include those useful for mutagenesis of the gene of interest or for expression of the gene of interest in host cells. Numerous expression systems are available for expression of the DNA. The expression of natural or synthetic nucleic acids encoding Ran and Ran-binding proteins is typically achieved by operably linking the DNA to a promoter (which is either constitutive or inducible) within an expression vector. By expression vector is meant a DNA molecule, linear or circular, that comprises a segment encoding a Ran or Ran-binding protein, operably linked to additional segments that provide for its transcription. Such additional segments include promoter and terminator sequences. Depending upon the intended host cell for expression, an expression vector also may include one or more origins of replication, one or more selectable markers, an enhancer, a polyadenylation signal, etc. Expression vectors generally are derived from plasmid or viral DNA, and can contain elements of both. The term “operably linked” indicates that the segments are arranged so that they function in concert for their intended purposes, for example, transcription initiates in the promoter and proceeds through the coding segment to the terminator. See Sambrook et al, supra.

The technique of long PCR has made in vitro propagation of large constructs possible, so that modifications to the gene of interest, such as mutagenesis or addition of expression signals, and propagation of the resulting constructs can occur entirely in vitro without the use of a replicating vector or a host cell. In vitro expression can be accomplished, for example, by placing the coding region for the Ran protein or Ran-binding protein in an expression vector designed for in vitro use and adding rabbit reticulocyte lysate and cofactors; labeled amino acids can be incorporated if desired. Such in vitro expression vectors may provide some or all of the expression signals necessary in the system used. These methods are well known in the art and the components of the system are commercially available. The reaction mixture can then be assayed directly for the Ran protein or Ran-binding protein for example by determining their activity, or the synthesized protein can be purified and then assayed.

The open reading frame encoding the Ran protein or Ran-binding protein or a functional portion thereof is operably joined to transcriptional and translational initiation and termination regions. These regions can be derived from a variety of sources, including the DNA to be expressed, genes known or suspected to be capable of expression in the desired system, expression vectors, and chemical synthesis. The choice of a promoter will depend in part upon whether constitutive or regulatable, e.g. inducible, expression is desired and/or whether it is desirable to produce the Ran and Ran-binding proteins at a particular stage of plant development and/or in a particular tissue. Numerous transcription initiation regions that provide for constitutive or inducible expression in a plant cell are known.

Among sequences known to be useful in providing for constitutive gene expression are regulatory regions associated with Agrobacterium genes, such as the genes encoding nopaline synthase (Nos), mannopine synthase (Mas), or octopine synthase (Ocs), as well as regions coding for expression of viral genes, such as the 35S and 19S regions of cauliflower mosaic virus (CaMV). The CaMV 35S and CaMV 19S promoters are described in U.S. Pat. No. 5,352,605 and related patent U.S. Pat. No. 5,530,196. U.S. Pat. No. 5,196,525 describes increased transcription efficiency of CaMV 35S and other promoters by incorporating duplicates of a transcription activating sequence of the promoter (e.g. double CaMV 35S) into the construct. Plant-derived constitutive promoters include two AHAS (ALS) promoters isolated from corn.(see U.S. Pat. No. 5,750,866) and an ALS promoter (ALS3) derived from Brassica napus (see U.S. Pat. No. 5,659,026). Constitutive promoters and regulatory elements can also be isolated from genes that are expressed constitutively or at least expressed in most if not all tissues of a plant. Such genes include, for example, ACT11 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPc1 from maize (GenBank No. X15596, Martinez et al. J. Mol. Biol. 208:551-565 (1989)), and Gpc2 from maize (GenBank No. U45855, Manjunath et al., Plant Mol. Biol. 33:97-112 (1997)). Other constitutive promoters include the rice actin 1 (Act 1) gene promoter (e.g., Wanggen Zhang et al. (1991) The Plant Cell 3:1155-1165) and the corn ubiquitin 1 gene (Ubi 1) promoter (e.g., Christensen et al. (1992) Plant Mol. Biol. 18:675-689). The term constitutive as used herein does not necessarily indicate that a gene is expressed at the same level in all cell types, but that the gene is expressed in a wide range of cell types, although some variation in abundance is often detectable.

Expression is targeted to a particular location within a host plant by using promoters that preferentially, preferably specifically, direct expression in one or more plant tissue and/or plant part and/or a particular cell type within a tissue or plant part. Promoters have been described for specific expression in fruit, sink organ, vasculature and phloem, epidermis, roots, seeds and flowers. See for example U.S. Pat. No. 5,463,174, U.S. Pat. No. 4,943,674, U.S. Pat. No. 5,106,739, U.S. Pat. No. 5,175,095, U.S. Pat. No. 5,420,034, U.S. Pat. No. 5,188,958, and U.S. Pat. No. 5,589,379. Other sequences can be identified from cDNA libraries using differential screening techniques, for example, or may be derived from sequences known in the literature.

In providing for transcription and/or expression preferentially in plant cells which function as carbon sinks, transcriptional initiation regions that provide for expression preferentially in specific tissue types, such as roots, tubers, seeds or fruit generally are used. Examples of transcriptional initiation regions that find use preferentially provide for transcription in certain tissues or under certain growth conditions, such as those from napin (see U.S. Pat. No. 5,608,152), seed or leaf ACP, the small subunit of RUBISCO, patatin (see U.S. Pat. No. 5,723,757; this promoter also is reported to express a marker gene in hairy roots, considered sink tissue, from sugar beets.), zein, and the like. Fruit specific promoters are also known, one such promoter is the E8 promoter, described in Deikman et al. (1988) EMBO J. 2:3315-3320; and DellaPenna et al. (1989) Plant Cell 1:53-63. Promoters directing root-specific expression are described in U.S. Pat. No. 5,635,618 and U.S. Pat. No. 5,633,363. Promoters that direct expression preferentially in seeds also can be considered as growth-stage specific. U.S. Pat. No. 5,677,474 describes a barley α-amylase gene promoter that expresses in seeds of oat and barley plants.

Promoters that direct expression of nucleic acids in ovules, flowers or seeds are particularly useful in the present invention. Such promoters may be, for example, ovule-specific, embryo-specific, endosperm-specific, integument-specific, seed coat-specific, or some combination thereof. Examples include a promoter from the ovule-specific BEL1 gene described in Reiser et al. Cell 83:735-742 (1995) (GenBank No. U39944). Other suitable seed specific promoters are derived from the following genes: MAC1 from maize (Sheridan et al. Genetics 142:1009-1020 (1996), Cat3 from maize (GenBank No. L05934, Abler et al. Plant Mol. Biol. 22:10131-1038 (1993), the gene encoding oleosin 18 kD from maize (GenBank No, J05212, Lee et al. Plant Mol. Biol. 26:1981-1987 (1994)), vivparous-1 from Arabidopsis (Genbank No. U93215), the gene encoding oleosin from Arabidopsis (Genbank No. Z17657), Atmyc1 from Arabidopsis (Urao et al. Plant Mol. Biol. 32:571-576 (1996), the 2s seed storage protein gene family from Arabidopsis (Conceicao et al. Plant 5:493-505 (1994)) the gene encoding oleosin 20 kD from Brassica napus (GenBank No. M63985), napA from Brassica napus (GenBank No. J02798, Josefsson et al. JBL 26:12196-1301 (1987), the napin gene family from Brassica napus (Sjodahl et al. Planta 197:264-271 (1995), the gene encoding the 2S storage protein from Brassica napus (Dasgupta et al. Gene 133:301-302 (1993)), the genes encoding oleosin A (Genbank No. U09118) and oleosin B (Genbank No. U09119) from soybean and the gene encoding low molecular weight sulphur rich protein from soybean (Choi et al. Mol Gen, Genet. 246:266-268 (1995)).

DNA encoding binding proteins capable of binding to a DNA regulatory sequence which regulates expression of one or more environmental stress tolerance gene in a plant can be used in conjunction with promoters which are known or are found to cause inducible transcription of the DNA into mRNA in plant cells can be used in the present invention. Such promoters can be obtained from a variety of sources such as plant and inducible microbial sources, and may be activated by a variety of exogenous stimuli, such as cold, heat, dehydration, pathogenesis and chemical treatment. The particular promoter selected is preferably capable of causing sufficient expression of the regulatory binding protein, such as CBF1, to enhance plant tolerance to environmental stresses. Examples of promoters which can be used include the promoter for the DRE (C-repeat) binding protein gene dreb2a (Liu, et al. Plant Cell 10: 1391-1406 (1998)) that is activated by dehydration and high-salt stress, the promoter for delta 1-pyrroline-5-carboxylate synthetase (P5CS) expression of which is induced by dehydration, high salt and treatment with plant hormone abscisic acid (ABA) (Yoshiba, et al., Plant J. 7 751-760 (1987)), the promoters for the rd22 gene from Arabidopsis transcription of which is induced by salt stress, water deficit and endogenous ABA (Yamaguchi-Shinozaki and Shinozaki, Mol Gen Genet 238 17-25 (1993)), the promoter for the rd29b gene (Yamaguchi-Shinizaki and Shinozaki, Plant Physiol., 101 1119-1120 (1993)) expression of which is induced by desiccation, salt stress and exogenous ABA treatment (Ishitani et al., Plant Cell 10 1151-1161 (1998)), the promoter for the rab18 gene from Arabidopsis transcripts of which accumulate in plants exposed to water deficit or exogenous ABA treatment, and the promoter for the pathogenesis-related protein 1a (PR-1a) gene expression of which is induced by pathogenesis, organisms or by chemicals such as salicylic acid and polyacrylic acid.

The promoters described above can be further modified to alter their expression characteristics. For example, the drought/ABA inducible promoter for the rab18 gene can be incorporated into seed-specific promoters such that the rab 18 promoter is drought/ABA inducible only when the plant is developing seeds. Similarly, any number of chimeric promoters can be created by ligating a DNA fragment sufficient to confer environmental stress inducibility from the promoters described above to produce promoters with other specificities such as tissue-specific promoters, developmentally regulated promoters, light-regulated promoters, hormone-responsive promoters, etc. This results in the creation of chimeric promoters that can be used to express regulatory binding proteins in any plant tissue or combination of plant tissues. Expression also can be made to occur either at a specific time during a plant's life cycle or throughout the plant's life cycle. See for example, U.S. Pat. No. 6,417,428.

The termination region can be derived from the 3 ′region of the gene from which the initiation region was obtained or from a different gene. A large number of termination regions are known to those of skill in the art and have been found to be satisfactory in a variety of hosts from the same and different genera and species. The termination region usually is selected more as a matter of convenience rather than because of any particular property.

Constructs containing the operably linked open reading frame and transcription initiation and termination regions can be introduced into a host cell by any of a variety of standard techniques, depending in part upon the type of host cell. These techniques include transfection, infection, bolistic impact, electroporation, microinjection, or any other method that introduces the gene of interest into the host cell. Microinjection techniques are known in the art and well described in the scientific and patent literature. The introduction of DNA constructs using polyethylene glycol precipitation is described in Paszkowski et al. Embo J. 3:2717-2722 (1984). Electroporation techniques are described in Fromm et al. Proc. Natl. Acad. Sci. USA 82:5824 (1985). Ballistic transformation techniques are described in Klein et al. Nature 327:70-73 (1987). See also for example U.S. Pat. No. 4,743,548, U.S. Pat. No. 4,795,855, U.S. Pat. No. 5,068,193, U.S. Pat. No. 5,188,958, U.S. Pat. No. 5,463,174, U.S. Pat. No. 5,565,346 and U.S. Pat. No. 5,565,347.

Preferably, transgenic plants are obtained using Agrobacterium tumefaciens-mediated transformation. The precise transformation procedure can be adapted for the plant of interest. For example, it may be based on co-culturing of Agrobacteria with root explants, hypocotyls, cotyledons, cotyledon segments, embryogenic callus, young leaf segments, bisections of seedling shoot apex, immature zygotic embryos, or cotyledon node regions. In some instances, transformation can be aided by wounding the tissue to be used for transformation, for example by caustic means or by cutting, prior to co-cultivation with Agrobacteria. The most frequently-cited reference for methods of plant transformation by the Agrobacteria system is Horsch et al. (1985) Science 227:1229-1231.

The Agrobacteria transformation system has some limitations. One is the relatively lengthy process of generating Agrobacteria with the desired DNA sequences in a Ti plasmid. The other is that, while use of Agrobacteria is generally effective at transforming dicotyledons, it is considered much less effective on monocotyledons. However, several monocotyledon species have been transformed using this method (e.g., U.S. Pat. No. 5,169,770, U.S. Pat. No. 5,149,645). U.S. Pat. No. 5,677,175 describes the transformation of three rice plant species by the Agrobacteria method. Soybean (Glycine max [L.] MERR) have been of the most recalcitrant crops to genetically engineer probably because of the difficulty in regenerating plants from specific cells of tissues once they are transformed. Soybean transformation methods have been reviewed recently by Widholm, In K. Wang, et al., eds., Transformation of plants and soil microorganisms, Cambridge University Press, 101-124 (1995), Finer et al. In Verma et al., eds. Soybean genetics, molecular biology and biotechnology, CAB International, Wallingford, UK 249-262 (1996), and Trick et al., Plant Tissue Cult. Biotech 3:9-26 (1997). The production of fertile transformed soybean plants was first reported about 10 years ago and since that time many successful efforts have been documented including some that have culminated in commercial products. The most popular methods in use today are Agrobacterium tumefaciens mediated transformation of the cotyledon node organogenic regeneration system and particle bombardment mediated transformation of an embryogenic suspension culture system. The first transgenic soybean plants using Agrobacterium-mediated transformation were obtained from cotyledon explants (Hinchee et al., Bio/Technol. 6:915-922 (1998)) although the transformation frequencies were low. An alternative technique where target tissues are subjected to pulses of ultrasound in the presence of Agrobacterium has also been reported (Trick et al., Plant Tissue Cult. Biotech 3:9-26 (1997); Trick and Finer, Plant Cell Reports 17:482-488 (1998); Samtarem et al., Plant Cell Rep 17:752-759 (1998); see also Yan et al, Plant Cell Reports 19:1090-1097 (2000), Annette et al, Plant Molecular Biology Reporter 18:51-59 (2000), and Donaldson and Simmonds, Plant Cell Reports 19:485-490).

For convenience, a host cell which has been manipulated by any method to take up a DNA sequence or construct is referred to as “transformed” or “recombinant” herein. The transformed host cell has at least have one copy of the expression construct and may have two or more, depending upon whether the gene is integrated into the genome, amplified, or is present on an extrachromosomal element having multiple copy numbers.

The transformed host cell can be identified by selection for a marker gene contained on the introduced construct that confers a selectable phenotype on plant cells. Alternatively, a separate marker construct can be introduced with the desired construct, as many transformation techniques introduce multiple DNA molecules into host cells. Typically, transformed hosts are selected for their ability to grow on selective media that incorporates an antibiotic. For example, the marker may encode biocide resistance, particularly antibiotic resistance, such as resistance to kanamycin, the amino glycoside G418 (see U.S. Pat. No. 5,034,322), bleomycin, hygromycin, or herbicide resistance, such as resistance to chlorosulfuron or Basta.

Selection of a transformed host cell also can occur when the expressed marker protein can be detected, either directly or indirectly. The marker protein can be expressed alone or as a fusion to another protein. The marker protein can be one which is detected by its enzymatic activity; for example β-galactosidase can convert the substrate X-gal to a colored product, and luciferase can convert luciferin to a light-emitting product. The marker protein can be one which is detected by its light-producing or modifying characteristics; for example, the green fluorescent protein of Aequorea victoria fluoresces when illuminated with blue light. Antibodies can be used to detect the marker protein or a molecular tag on, for example, a protein of interest. Cells expressing the marker protein or tag can be selected, for example, visually, or by techniques such as FACS or panning using antibodies.

Transformed plant cells which are derived by any of the above transformation techniques can be cultured to regenerate a whole plant which possesses the transformed genotype and thus the desired phenotype such as increased biomass and/or seed mass. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration also can be obtained from plant callus, explants, organs, or parts thereof. Such regeneration techniques are described generally in Klee et al. Ann. Rev. Plant Phys. 38:467-486 (1987).

Genetic transformation with the desired construct can be confirmed by any of a number of methods, including genomic PCR, RT-PCR, Southern, Northern, or Western blot analysis. The primers, probes, or antibodies necessary to practice these methods can be obtained using any means known to the art. Plants produced according to the invented method can be polyploid, preferably tetraploid. Polyploid plants comprise cells with three or more chromosome sets. The number of chromosome sets of the transformants can be determined by any known suitable method, including Giesma staining and chromosome counting, or flow cytometry. Genetic stability of the polyploid status of different generations of transformants can be similarly ascertained.

The transformed plant cells are grown in appropriate nutrient medium to provide for selected calli, where plant cells or protoplasts have been modified. After transformation of cells or protoplasts, the choice of methods for regenerating fertile plants generally is not particularly important. Suitable protocols are available for Leguminosae (alfalfa, soybean, clover, etc.), Umbelliferae (Carrot, celery, parsnip), Cruciferae (cabbage, radish, rapeseed, broccoli, etc.), Curcurbitaceae (melons and cucumber), Gramineae (wheat, corn, rice, barley, millet, etc.), Solanaceae (potato, tomato, tobacco, peppers, etc.), and various other crops. See protocols described in Ammirato et al. (1984) Handbook of Plant Cell Culture—Crop Species. Macmillan Publ. Co. Shimamoto et al. Nature 338:274-276 (1989); Fromm et al., Bio/Technology 8:833-839 (1990); Vasil et al. Bio/Technology 8:429-434 (1990). Once the calli has formed, the medium may then be changed to encourage root and shoot formation and the resulting shoots transferred to appropriate growth medium for growth of plants. When the plants have been grown to the desired stage, the plants or plant parts, for example, seeds, fruit or the like, are harvested and any desired product isolated in accordance with conventional ways. The plant may be ground and extracted with appropriate solvents, chromatographed, crystallized, solvent extracted, etc. The crude product may then be purified in accordance with the nature of the product.

Thereafter, the plant may be regenerated from seeds, so that the process of regeneration from calli need not be repeated and all the progeny plants maintain the same number of chromosomes as the parent plants, such as tetraploidy status. Once the expression cassette is stably incorporated in the transgenic plants and confirmed to be operable, it can be introduced into other plants by sexual crossing. Any of a number of standard breeding techniques can be used, depending upon the species to be crossed. Since transgenic expression of the nucleic acids of the invention leads to phenotypic changes in seeds and fruit, plants comprising the expression cassettes are sexually crossed with a second plant to obtain the final product. The seed of the invention can be derived from a cross between two transgenic plants of the invention, or a cross between a plant of the invention and another plant which is of the same ploidy as the transgenic plant or as appropriate self-crossing in the same plant. The tetraploid plants also can be asexually produced by a meristem tissue culture process. Portions of the plant part or meristems comprised of unspecialized cells, capable of later differentiation, are cut from the plant and developed, under carefully controlled sterile conditions into new individual plants.

Increasing seed size and preferably other components of the seed such as protein, amino acid, and/or oils content is particularly desirable in crop plants in which seed are used directly for animal and/or human consumption or for industrial purposes. Examples include soybean, canola, and grains such as rice, wheat, corn, rye, and the like. Seedless varieties can be produced by crossing a tetraploid plant with a diploid plant; this feature is particularly desirable in plants grown for their fruit and in which large seeds may be undesirable. Examples include cucumbers, tomatoes, melons, and cherries.

Seed obtained from plants of the present invention is analyzed according to well known procedures to identify seed with the desired trait. Increased size can be determined by weighing seeds or by visual inspection. Usually, the seed mass is at least about 10%, often about 20% greater than the average seed mass of plants of the same variety that lack the expression cassette. The mass can be about 50% greater and preferably at least about 75% to about 100% greater. Increases in other properties e.g., protein and oil usually is proportional to the increases in mass. Thus, in some embodiments protein, oil and/or secondary metabolite content can be increased by about 10%, 20%, 50%, 75% or 100%, or in approximate proportion to the increase in mass. Protein content is conveniently measured by the method of Bradford et al. Anal. Bioch. 72:248 (1976). Oil content is determined using standard procedures such as gas chromatography. These procedures also can be used to determine whether the types of fatty acids and other lipids are altered in the plants of the invention.

Where it is desired to produce a compound of interest, either a compound naturally produced by the original diploid plant or a newly introduced synthetic capability, the polyploid plants also can be used as a source of material for preparation of cell cultures in which individual cells have the tetraploid genotype and the large phenotype. The cells to be cultured can be initiated from a variety of explants, for example, a leaf, style, anther or stem of a transformed plant that has been shown to be polyploid, segments of which can be placed on solid plant culture medium. Callus cells can proliferate from any of the tissues of these organs and the callus cells can then be transferred to liquid suspension culture. Alternatively, seeds can be surface sterilized, and placed in a solid or liquid plant tissue culture medium to initiate germination. The germinating seedlings can then be maintained, for a time, in liquid suspension culture. The suspension culture medium can be any known suitable medium such as MS medium (Mirashige, T. & Skoog, F. (1962) Physiologia Plantarum 15:473-497; Wu, M. & Wallner, S. (1983) Plant Physiol. 72:817-820) or variants thereof. The suspension culture medium can be optimized for enhanced cell growth and/or enhanced production of the compound of interest. Transfer to suspension culture is preferred because in general it increases production and because it is possible to scale up a liquid suspension culture. Air fermentors are preferred because they reduce shear stress on the cells. While cells can produce the compound of interest on a solid medium, mass culture on solid media poses a number of practical difficulties, including collection of product.

Usually, a plant cell hormone is employed to enhance growth of the cells in culture and/or production of the compound of interest. Plant hormones that can be used include, for example, the auxins such as 2,4-dichlorophenoxyacetic acid (2,4-D), naphthoxyacetic acid (NOA) and 2,4-dichlorophenoxybutyric acid (2,4-DB) or mixtures thereof. The use of given hormone may provide improved callus or cell growth for a given plant cell. Plant cell lines can be adapted using methods well-known in the art to exhibit good growth on lower levels of (or no) plant hormones. The use of lower hormone levels in suspension culture can decrease manufacture cost and alleviate the potentially detrimental effects of hormones on plant cells. The desired product can be recovered from the culture medium by methods well known in the art. Of interest for production in cultured cells are plant pharmaceuticals such as taxol, genistein, diadzein, codeine, morphine, quinine, shikonin, ajmalacine and serpentine and products that are used in foods such as anthocyanins, saffron, vanilla, and a wide variety of other fruit and vegetable flavors and texture modifying agents.

An example of the use of the tetraploid cells in a culture system is for large scale preparation of paclitaxel. Natural paclitaxel was originally isolated from the bark of Taxus brevifolia. This species is slow growing, taking over a hundred years for a young yew to mature. More importantly, paclitaxel occurs in low concentrations, (0.002 to 0.04% per dry weight), primarily in the inner bark of the tree. Deforestation of this particular yew is an obvious concern and poses a problem for replenishing the limited supply of naturally occurring paclitaxel. Examples of taxane-type diterpene-producing plants that can be used as a source of cells for transformation and growth in cell culture include Taxus plants such as European yew (Taxus baccata LINN), Japanese yew (T. cuspidata SIEB. et ZUCC), Kyaraboku (T. cuspidata SIEB. ET ZUCC var. nana REHDER), Pacific yew (T. brevifolia NUTT), Canadian yew (T. canadensis MARSH), Chinese yew (T. chinensis), Himalayan yew (T. wallichiana ZUCC) and T. media. T. baccata LINN and T. media are particularly preferable. For transformation, an explant is taken from a part of the Taxus plant such as a root, growing point, leaf, stem, seed or the like, sterilized, placed on a suitable medium such as Woody Plant Medium and the cells transformed by any convenient method with a construct such as an antisense construct containing the open reading frame from the ATRan1b gene or a homolog thereof. Following transformation, the cells are cultured using methods known to those of skill in the art. The paclitaxel and/or other related compounds of interest are then isolated and purified. A process for the purification of paclitaxel and/or cephalomannine and/or 110-DAB III and/or 9-DHAB III from biomaterials is described for example in U.S. Pat. No. 6,469,186. A series of extractions, separations, and purifications provides commercial quantities with high purity of these products.

The methods of the invention can be used to confer polyploidy on essentially any plant. Thus, the invention has use over a broad range of plants, both monocotyledenous and dicotelydenous, including species from the genera Anacardium, Arachis, Asparagus, Atropa, Avena, Brassica, Citrus, Citrullus, Capsicum, Carthamus, Cocos, Coffea, Cucumis, Cucurbita, Daucus, Elais, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannesetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, and Zea.

Tomato and Arabidopsis have been generally used as model plants in the field of plant molecular biology. In the present invention, transgenic tomatoes or Arabidopsis are generated by transformation of recombinant vectors overexpressing or suppressing Ran or Ran-binding proteins. The recombinant vectors include any gene-expressing promoters that could express sense or antisense sequences of Ran or Ran-binding proteins, or sense and antisense sequences of the protein genes at the same time.

To generate the transgenic plants, PsRan (SEQ.ID.NO:1) which is a Ran protein of Pisum sativum was cloned from an etiolated pea plumule library and was inserted into a pLBJ21 vector to construct a recombinant vector pLBJ21/PsRan. The pBJ21 vector is a pKYLX71 derivative plasmid in which a HindIII recognition site in the multi-cloning region of a pKYLX71 expression cassette is converted to an EcoRI recognition site (Schardl et al (1987) Gene 61,1-11, 1987; Lloyd, et al (1992) Science 258,1773-1775). It is a binary vector, and it is used as a transformation vector in the present invention. In addition, AtRanBP1b (SEQ.ID.NO:2) and AtRanBP1c (SEQ.ID.NO:3) were inserted into pLBJ21 vectors. The three recombinant vectors pLBJ21/PsRan, pLBJ21/AtRanBP1b and pLBJ21/AtRanBP1c were transformed into Arabidopsis cells and plants regenerated from the transformed cells. Transgenic lines of the resulting plants were selected to characterize transgenic phenotypes. Transgenic pLBJ21/PsRan Arabidopsis showed a long root and big plant phenotype. Transgenic Arabidopsis transformed with pLBJ21/AtRanBP1b showed a big plant phenotype. Transgenic Arabidopsis transformed with pLBJ21/AtRanBP1c showed a long-root, increased seed weight, and big plant phenotype.

In addition, in order to generate transgenic plants in which genes involved in Ran-mediated cellular processes are suppressed, antisense base sequences to AtRanBP1b (SEQ.ID.NO:4) and AtRanBP1c (SEQ.ID.NO:5) were respectively designed. Transgenic plants in which Ran-mediated cellular processes are suppressed can be generated with recombinant vectors having either a whole antisense sequence AtRanBP1b or AtRanBP1c, or a part of those base sequences sufficient to provide for suppression. Nucleotide sequences generally are 50 base pairs (bp) or more in length to be effective. In the case where the sense and antisense base sequences of genes are simultaneously expressed and suppress endogenous genes involved in Ran-mediated cellular processes in the transgenic plants, it is desirable to have the sequences preferably 26 bp or more in length (Parrish et al., 2000 Molecular Cell 6: 1077-1087; Chuang et al., Proc. Natl. Acad. Sci, USA 97: 4985-4990).

In the present invention, antisense AtRanBP1b or AtRanBP1c were inserted into pLBJ21 vectors to construct two kinds of recombinant vectors, pLBJ21/antisense AtRanBP1b and pBLJ21/antisense AtRanBP1c. These were transformed into Arabidopsis cells used Agrobacteria as a vector. Plants were regenerated from the transformed plant cells and the phenotype of the tranformants was observed. Transgenic Arabidopsis that was transformed with the recombinant vector pLBJ21/antisense AtRanBP1b (KCTC 0850BP) showed a big plant phenotype; virtually, sizes of all plant bodies are increased. For example, the leaf area, stem thickness, the volume and weight of the seed, height of plant, size of flower, length of trichome, and the number of seeds per plant were significantly increased. Transgenic Arabidopsis that was transformed with the recombinant vector PLBJ21/antisense AtRanBP1c (KCTC 0851 BP) showed the phenotypes of long primary root growth, and the same big-plant as described above.

Sensitivity to auxin in the Arabidopsis plants transformed with the pLBJ21/antisense AtRanBP1c was tested. Auxin is a plant hormone that promotes germination of pollen, extension of the pollen tube, formation of lateral roots and flower buds, generation of sprouts or roots from callus, and the division and growth of the dividing cells. In the present invention, it was tested whether or not the sensitivity to auxin, that affects the division and growth of cells, is changed in the transformed plants. In general, a low concentration (10⁻⁶-10⁻⁷M) of auxin in the roots promotes the division of root cells and subsequent root growth while inhibiting the growth of lateral roots, whereas a high concentration (10⁻⁶-10⁻⁵ M) of auxin acts in the opposite manner.

Transgenic Arabidopsis transformed with pBLJ21/antisense AtRanBP1c of the present invention shows a hyper-sensitive response to auxin; even a low concentration of auxin such as pM supplied from the outside suppressed root growth and promoted the initiation of lateral root differentiation. In contrast in the wild-type plants, there was no response to the same concentration of auxin. In wild type plants, the lateral root differentiation was promoted at 10⁻⁷ M of auxin. Since the transgenic plants of the present invention have increased auxin sensitivity, a very low concentration of endogenous auxin, that does not generally promote root growth in wild types, does in fact promote root growth in plants genetically modified by the present invention.

In addition, the methods of this invention were used to generate tomatoes transformed with pLBJ21/antisense AtRanBP1b (KCTC0850BP), pBLJ21/antisense AtRanBP1c (KCTC0851 BP), pBLJ21/PSRan (KCTC0837BP), pBLJ21/AtRanBP1b (KCTC0838BP), or pLBJ21/AtRanBP1c (KCTC0839BP).

The invention is useful in a variety of applications. Essentially, any method or product which is based on plant parts may benefit from the invention. Examples of applications possible include super-productive agricultural crops, ornamental plants with improved aesthetic qualities, paper production based on sugar cane pulp, ethanol production based on corn grain, cold-tolerant sweet potatoes, phytoremediation, erosion control using long-rooted grasses, crops with increased cotton yield and fiber quality parameters, and improved varieties of ginseng and cassaya. Unlike prior tetraploid generation methods, tetraploid varieties that are both fertile and super-productive can be consistently made according to the present invention. Moreover, the presented techniques of plant cell culture based on cells form tetraploid plants allow achievement of desirable growth rates and product yield, and thus increased productivity.

The present invention will be explained in more detail with reference to the following Examples; the Examples are offered by way of illustration, not limitation.

### EXAMPLES

Biological Deposits.

** pLBJ21/PsRan was deposited in the Korean collection for type cultures, as KCTC 0837BP, pLBJ21/antisense AtRanBP1b as KCTC 0850BP, pLBJ21/antisense AtRanBP1c as KCTC 0851BP, pBLJ21/AtRanBP1b as KCTC 0838BP), pLBJ21/AtRanBP1c as KCTC 0839BP.

### Example 1

### Transgenic Plants Expressing PsRan Protein

(1) The Cloning of PsRan Gene

Using an EcoRI/XhoI gene segment of an ATTS1902 clone provided from Arabidopsis Cdna BANK (ARBC, Ohio) as a probe, a pea plumule cDNA expression library was screened to obtain PsRan genes.

(2) The Construction of a Recombinant Vector Containing PsRan Gene

PsRan cDNA was cloned at EcoRI and XhoI sites in a pBluescript SK+ (Strategene, Calif.) vector. The genes were digested with the restriction enzymes EcoRI and XhoI, and then were cloned into a pLBJ21 vector that had been digested with the same restriction enzymes to construct a pLBJ21/PsRan expressing PsRan under a CaMV35S promoter, as shown in FIG. 1. The fusion vector was introduced into Agrobacterium tumefaciens GV3101 containing pMP90 plasmid by electroporation (Koncz, C., and Schell, J. (1986) Mol. Gen. Genet. 204, 383-396) to transform the recombinant vector into plants.

(3) The Generation of Arabidopsis Transformed With pLBJ21/PsRan

The pLBJ21/PsRan was introduced into root explants of Arabidopsis using Agrobacterium-mediated transformation method (Valvekens et al., D., Montagu, M. V., and Lijsebettens, m. v. (1988) Proc. Natl. Acad. Sci. USA 85,5536-5540). T1 seeds from primary transformants were grown on a Germination Medium (Valvekens et al., D., Montagu, M. V., and Lijsebettens, M. V. (1988) Proc. Natl. Acad. Sci. USA 85,5536-5540) and the transformants were selected with 50 ug/L of kanamycin. T2 seeds were recovered from the selected T1 plants, and were grown on the same medium to identify homozygous T2 seeds. Identified T2 seeds were used to observe the characteristics of the transgenic plants.

### Example 2

### Transgenic Plants Expressing AtRanBP1b Protein

(1) The Cloning of AtRanBP1b Gene

A yeast two-hybrid screening method was used to clone Ran-binding proteins in Arabidopsis cDNA library using PsRan as a bait. Specifically, a full length PsRan was amplified by the PCR method using a primer of SEQ.ID.NO:3 containing an initial ATG codon and a primer of SEQ.ID.NO:4 containing a stop codon. The amplified PsRan was digested with EcoRI and BamHI, and then was subcloned into a pGBT9 vector (Clontech, CA) predigested with the same restriction enzymes. A recombinant pGBT9/PsRan vector was cotransformed into Saccharomyces cerevisiae Y190 together with an Arabidopsis cDNA library that was subcloned in pACT, and then a Ran binding protein, AtRanBP1b, was cloned according to the method described in Yeast Two Hybrid Screening published in Clontech.

(2) The Construction of a Recombinant Vector, pLBJ21/AtRanBP1b, Containing AtRanBP1b Gene

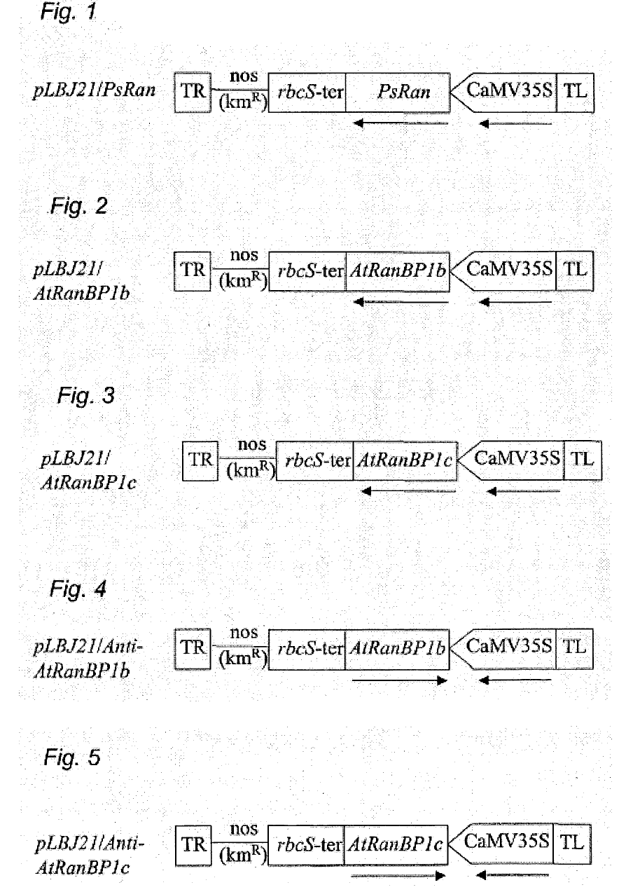

A pLBJ21/AtRanBP1b recombinant vector was constructed by the same method as described in Example 1, except that AtRanBP1b was used instead of PsRan, as shown in FIG. 2. Specifically, a full length AtRanBP1b was PCR amplified using the primer of SEQ.ID.NO:8 comprising ATG and the primer of SEQ.ID.NO:9, and then the amplified genes were digested with XhoI/XbaI. The digested AtRanBP1b fragment was subcloned in pLBJ21 that was digested with the same enzymes to construct a recombinant vector pLBJ21/AtRanBP1b as shown in FIG. 2. The recombinant vector was introduced into Agrobacterium by electroporation as mentioned above.

(3) The Generation of Arabidopsis Transformed With pLBJ21/AtRanBP1b

Arabidopsis plants that are transformed with pJBL21/AtRanBP1b were generated by the same method as described in Example 1.

### Example 3

### Transgenic Plants Expressing AtRanBP1c Gene

(1) The Cloning of AtRanBP1c Gene

AtRanBP1c was cloned by the yeast two-hybrid screening method as described in Example 2 (1).

(2) The Construction of a Recombinant Vector, pLBJ21/AtRanBP1c, Containing AtRanBP1c Gene

A pLBJ21/AtRanBP1c recombinant vector was constructed by the same method as described in Example 2 (2), except that AtRanBP1c was used instead of AtRanBP1b, as shown in FIG. 3. A full-length AtRanBP1c was PCR amplified using the primer of SEQ.ID.NO:10 and the primer of SEQ.ID.NO:11.

(3) The Generation of Arabidopsis Plants Transformed With pLBJ21/AtRanBP1c Arabidopsis plants transformed with pLBJ21/AtRanBP1c were generated by the same method as described in Example 1.

### Example 4

### Transgenic Plants Expressing Antisense AtRanBP1b

(1) The Preparation of an Antisense AtRanBP1b Base Sequence

A full length AtRanBP1b was amplified by RT-PCR from Arabidopsis total RNA, using primer 1 of SEQ.ID.NO:12 that has an XhoI enzyme recognition site in 3′-terminal cDNA of AtRanBP1b and primer 2 of SEQ.ID.NO:13 that has an XbaI recognition site in 5′-terminal cDNA

(2) The Construction of a Recombinant Vector, pLBJ21/antisense

AtRanBP1b, containing an antisense AtRanBP1b base sequence The RT-PCR amplified AtRanBP1b was digested with XhoI/XbaI to prepare a DNA fragment having an antisense AtRanBP1b base sequence in the direction from XhoI to XbaI. Then, the DNA fragment was cloned into the region of pLBJ21 digested with XhoI/XbaI to construct a pLBJ21/antisense AtRanBP1b overexpressing an antisense AtRanBP1b under a CaMV35S promoter, as shown in FIG. 4. The pLBJ21/antisense AtRanBp1b was introduced into Agrobacterium tumefaciens GV3101 containing pMP90 plasmid by electroporation (Koncz, C., and Schell, J. (1986) Mol. Gen. Genet. 204,383-396).

(3) The Generation of Transgenic Plants Transformed With pLBJ21/Antisense AtRanBP1b

Transgenic plants transformed with PLBJ21/antisense AtRanBP1b were generated by the same method as described in Example 1.

### Example 5

### Transgenic Plants Expressing Antisense AtRanBP1c

(1) The Preparation of an Antisense AtRanBP1c Base Sequence

A full length AtRanBP1c was RT-PCR amplified by the same method as described in Example 4 (1). Primers of SEQ.ID.NO:14 and SEQ.ID.NO:15 were used in RT-PCR.

(2) The Construction of a Recombinant Vector Containing an Antisense AtRanBP1c Base Sequence

A pLBJ21/antisense AtRanBP1c recombinant vector was constructed by the same method as described in Example 4 except that AtRanBP1c was used instead of AtRanBP1b, as shown in FIG. 5.

(3) The Generation of Arabidopsis Transformed With pLBJ21/Antisense AtRanBP1c

Arabidopsis plants transformed with pLBJ21/antisense AtRanBP1c were generated by the same method as described in Example 1.

### Examples 6 to 10

A pLBJ21/PsRan, pLBJ21/AtRanBP1b, pLBJ21/AtRanBP1c, pLBJ21/antisense AtRanBP1b, and pLBJ21/antisense AtRanBP1c recombinant vector were transformed into tomatoes by the same method as described in Examples 1 to 5 except a cotyledone of tomatoes was used instead of Arabidopsis root explants.

Experiment 1. The Identification of Transgenic Plants

(i) Genomic Southern Analysis

In order to identify whether or not the genes that were transformed into transgenic plants of the above Examples were stably introduced into the chromosomes of the plants, a Southern analysis was conducted with the genomic DNA of the transgenic plants.

Genomic DNA was isolated from 3 week-old plants, and it was digested with EcoRI. The digested DNA were separated on a 0.8% agarose gel by electrophoresis and transferred to a zeta-probe membrane (Biorad). The membrane was preincubated in 0.25 M sodium phosphate (PH 7.2) and 7% SDS at 65 C for 30 minutes. [α-³²P] dATP-CaMV 35S promoter (a fragment of pB1221 vector digested with XbaI/HindIII) probe was added in the incubating solution and the membrane was further incubated at 65 C for 20 hours. After the incubation, the membrane was washed with a solution containing 20 mM sodium phosphate, pH 7.2, and 5% SDS, and finally with the same solution containing 1% SDS at 65C for 1 hour. Then the membrane was exposed to a film to detect the inserted tansgene. As a result, transformed plants carrying CaMV35S promoter and the transgene were identified.

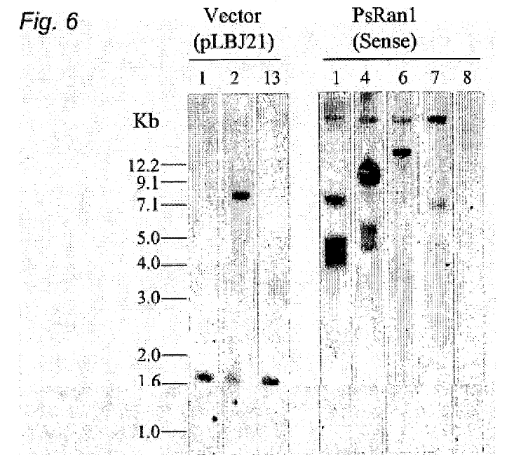

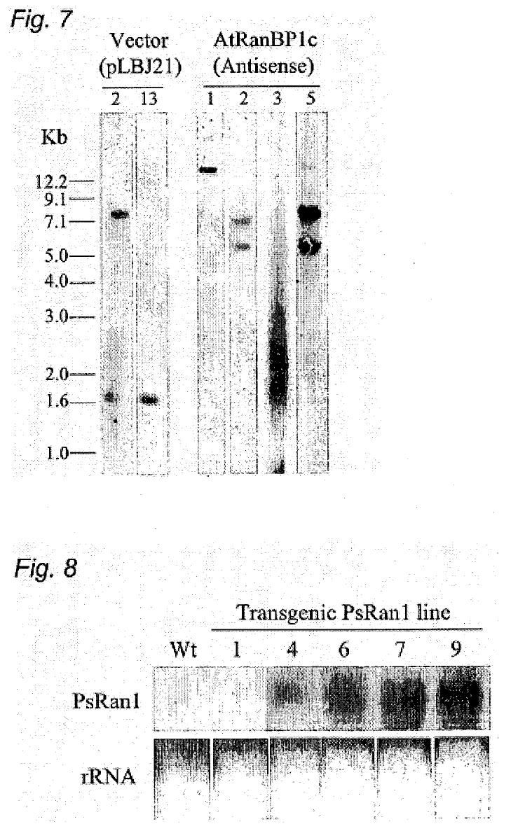

FIGS. 6 and 7 shows the Southern blotting analysis of the two different transgenic lines.

FIG. 6 shows a photograph of the DNA gel blot analysis (Southern blot analysis) of Arabidopsis transformed with pLBJ21/PsRan, and FIG. 7 shows a photograph of the DNA gel blot analysis of Arabidopsis transformed with pLBJ21/antisense AtRanBP1c. As shown in FIGS. 6 and 7, we generated many pLBJ21/PsRan and pLBJ21/antisense AtRanBP transgenic plants whose transgenes were inserted into the different chromosomes. (For example, CaMV35S fragments were identified by different bands with different length in the genome of Sense PsRan-1,-4,-6,-7,-8 plants).

(ii) Northern Blotting Analysis of RNA

In order to identify whether or not the transgenic plants, in which the introduction of genes was confirmed as shown above, express the introduced genes as well, they were further subjected to a Northern blotting analysis.

To do this, RNA of transgenic plants were extracted using a Trizol reagent (Gibco BRL) solution and separated on a 1.2% agarose gel containing 6% formaldehyde by electrophoresis. Northern blotting analysis was then conducted using the sense or antisense riboprobe of PsRan, AtRanBP1b or AtRanBP1c. The probe was prepared using a transcription kit, MAXIscript T3/T7 (Ambion, Tex.). After Northern hybridization, RNA was exposed to a intensifying screen and developed to measure the expression of corresponding RNAs.

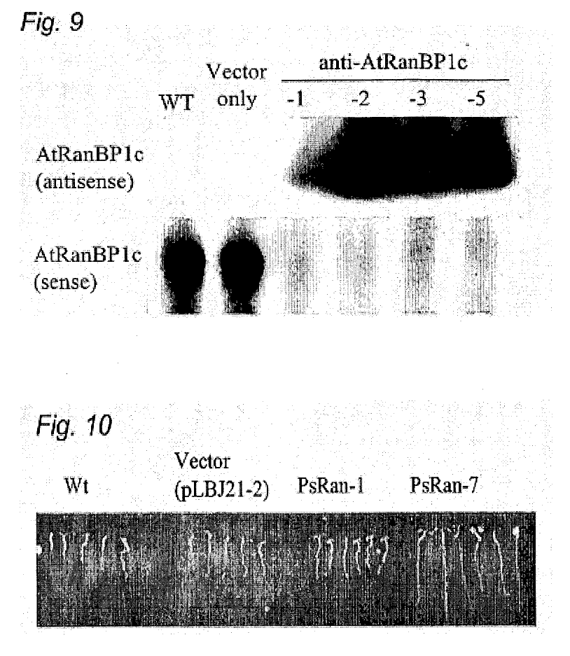

FIG. 8 shows a photograph of Northern blotting analysis of the plants transformed with pLBJ21/PsRan, and FIG. 9 shows a photograph of Northern blotting analysis for the plants transformed with pLBJ21/antisense AtRanBP1c. As shown in FIGS. 8 and 9, transgenes were overexpressed in the transgenic Arabidopsis plants. In addition, it was verified that antisense AtRanBP1c was expressed, and the expression of endogenous AtRanBP1c was inhibited in the corresponding transgenic plants, as shown in FIG. 9.

Experiment 2. The Phenotypes of 5 Different Transgenic Arabidopsis Lines

(i) The Phenotypes of a Root Length

The length of Arabidopsis roots according to Examples 1 to 5 was measured, and the results are shown in Tables 1 and 2 below.

TABLE 1
Effect Sense Constructs on Length of Arabidopsis Roots

| | Mean root length (mm) |
|---|---|
| Wild type | 5 ± 1.5 |
| pLBJ21/PsRan-1 | 6 ± 1.8 |
| pLBJ21/PsRan-4 | 15 ± 4 |
| pLBJ21/PsRan-7 | 13 ± 3.6 |

TABLE 2
Effect of Antisense Constructs on Length of Arabidopsis Roots

| | Mean root length (mm) |
|---|---|
| Wild type | 7.5 ± 1.5 |
| pLBJ21/antisense AtRanBP1c-1 | 12 ± 2.3 |
| pLBJ21/antisense AtRanBP1c-2 | 20 ± 4.1 |
| pLBJ21/antisense AtRanBP1c-3 | 18 ± 4.3 |
| pLBJ21/antisense AtRanBP1c-5 | 9 ± 1.2 |

As shown in FIG. 8, Table 1, and Table 2, the length of roots of the transgenic Arabidopsis (pLBJ21/PsRan-4) and the transgenic Arabidopsis (pLBJ21/PsRan-7) overexpressing PsRan was greater than that of the wild type plants or the transgenic Arabidopsis (pLBJ21/PsRan-1) that do not overexpress the PsRan gene. In addition, the root of the Arabidopsis transformed with pLBJ21/antisense AtRanBP1c was also longer than that of wild type plants.

FIG. 10 shows a photograph of roots of Arabidopsis transformed with pLBJ21/PsRan-7, and other control plants. The length of transgenic pLBJ21/PsRan-7 roots is greater than that of wild type or the Arabidopsis transformed with pLBJ21.

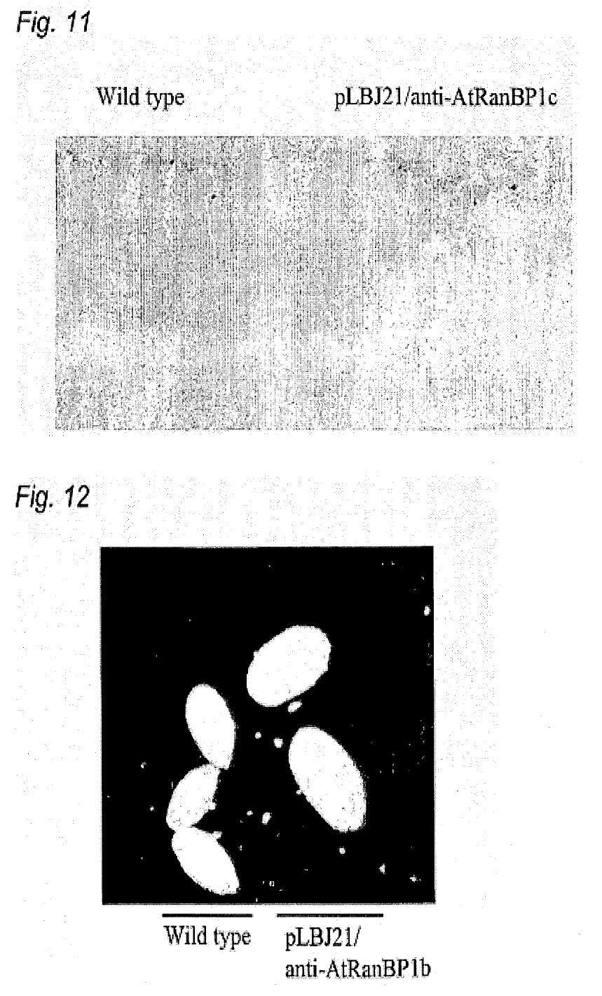

FIG. 11 shows a photograph of a root length of Arabidopsis transformed with pLBJ21/antisense AtRanBP. It is clear that a root length of Arabidopsis transformed with pLBJ21/antisense AtRanBP1c is greater than that of wild type.

For anatomical analysis of 5 different transgenic Arabidopsis lines of the above Examples, root tissues were fixed with Navishins solution (Mauseth, J. D., Montenegro, G., and Walckowiak, A. M. (1984) Can. J. Bor. 62,847-857). After the fixation, the plants tissues were dried through the standardized ethanol step. The ethanol was exchanged with xylene before being introduced into paraffin, and then the tissues were cut to 7 am and were stained to observe with a standard bright microscope. Each root was measured every 24 hours using a microscope of 40× magnification for anatomic use, and a ruler. The mean apical growth rate was calculated from the growth means of transgenic plants or wild-type plants.

A statistical analysis was conducted using a SAS program, and the measurement was conducted with micrometer scale rulers in the objective lens of 6.3× magnification. A wild-type Arabidopsis was analyzed for comparison.

The following Table 3 presents the microscopic anatomical difference between one of the transgenic plants, pLBJ21/antisense AtRanBP1c, and wild types.

TABLE 3
Microscopic Anatomical Difference Between Transgenic
Plant pLBJ21/Antisense AtRanBP1c and Wild Type

| | Mean root length (mm) | % wild type |
|---|---|---|
| Example 5 | 9.0 | 164 |

Mean root length of the transgenic plant transformed with pLBJ21/antisense AtRanBP1c in the above Table 3 was approximately 1.6 times longer than that of the wild type. This long root phenotype is due to an increase in mean length of the epidermis, cortex and endodermis, as can be seen in Table 4.

TABLE 4
Length of the Epidermis, Cortex and Endodermis of Transgenic
Plant pLBJ21/Antisense AtRanBP1c and Wild Type

| | |
|---|---|
| Mean cell length (μm) | Mean cell width (μm) |

| | Example 5 | | Example 5 | |
|---|---|---|---|---|
| | (pLBJ21/ | % wild | (pLBJ21/ | % wild |
| Cell | antisense | type | antisense | type |
| tissues | AtRanBP1c) | plants | AtRanBP1c) | plants |
| Epidermis | 157.0 | 173 | 14.8 | 103 |
| Cortex | 146.0 | 151 | 28.0 | 116 |
| Endodermis | 107.0 | 130 | 15.9 | 98 |

TABLE 5
Growth Rate of Root Apical Cells

| | Increased length (μm)/day | % wild type Example |
|---|---|---|
| Wild type | 505 | 100 |
| Example 5 | 750 | 149 |

In addition, as seen in Table 5, the growth rate of root apical cells of transgenic plants is approximately 1.5 times that of wild types.

(ii) Sensitivity of Transgenic Plants to IAA

In order to examine the response to IAA (indole acetic acid) of transgenic plants, seeds of transgenic Arabidopsis (pLBJ21/antisense AtRanBP) were surface-sterilized with a common bleaching solution for 15 minutes. They were then washed with sterile water, and then dried in the air. Plates for germination media (GM-sucrose) were prepared and seeds were placed therein, and incubated at 4 C for 72 hours so that germinations would occur simultaneously. Various concentrations of IAA's were added to the plates and the media were transferred to growth chambers and grown under all same conditions. Seeds were germinated and grown for 10 days (Modified from Knee and Hangarter, 1996). After 10 days, the lengths and shapes of roots were measured and observed. Those of the transgenic plants of Examples 1 to 5 were compared with those of wild-type plants, and the results are presented in the following Table 6.

TABLE 6
Effect of Auxin on Mean Root Length

| | Mean root length | Mean root length of |
|---|---|---|
| Auxin | of Example 5 ± | wild type; Average root |
| (M) | standard deviation (mm) | length of vector controls (mm) |
| 0 | 19.5 ± 0.9 | 9.6; 7.3 |
| 10⁻¹⁰ | 7.5 ± 3.9 | 9.1; 10.8 |
| 10⁻¹¹ | 8.2 ± 1.8 | 6.5; 8.1 |
| 10⁻¹² | 8.7 ± 2.7 | 6.4; 11.8 |
| 10⁻¹³ | 14.5 ± 3.1 | 6.8; 6.7 |
| 10⁻¹⁴ | 13.3 ± 1.9 | 7.6; 11.0 |

In the roots of wild-type plants, root growth generally was promoted by the application of 10⁻⁵ M to 10⁻⁶ M of auxin, and it was suppressed by a concentration exceeding the above range. As seen in Table 6, the sensitivity of the transgenic plants to auxin increased. Therefore, the growth of roots was suppressed by the application of even 10⁻¹⁰M to 10⁻¹² M of auxin. The suppression of root growth was caused by failure of the somatic cell divisions that occur in the dividing cells of the transgenic plants in Example 5. From this, it can be seen that the growth of primary roots in the transgenic plant of Example 5 was promoted by a low concentration of endogenous auxin (less than 10⁻¹¹M to 10⁻¹² M).

(iii) Seed Size Comparison of Transgenic Plants

The seeds of the T3 generation of 5 different transgenic lines according to Examples 1 to 5 were harvested and weighed. The results are presented in Table 7.

TABLE 7
Increased Weight of Seeds from Transgenic Plants

| Transgenic plants | Weight/100 seeds | Standard deviation |
|---|---|---|
| Wild type | 1.76 × 10⁻⁵ g | 1.69 × 10⁻⁶ |
| pLBJ21/PsRan-1 | 2.06 × 10⁻⁵ g | 1.67 × 10⁻⁶ |
| pLBJ21/PsRan-2 | 2.08 × 10⁻⁵ g | 8.37 × 10⁻⁷ |
| pLBJ21/PsRan-3 | 2.08 × 10⁻⁵ g | 1.64 × 10⁻⁶ |
| pLBJ21/PsRan-4 | 2.00 × 10⁻⁵ g | 1.87 × 10⁻⁶ |
| pLBJ21/PsRan-5 | 2.35 × 10⁻⁵ g | 2.16 × 10⁻⁶ |
| pLBJ21/PsRan-6 | 2.24 × 10⁻⁵ g | 1.82 × 10⁻⁶ |
| pLBJ21/antisense | 2.20 × 10⁻⁵ g | 2.74 × 10⁻⁶ |
| AtRanBP1c-1 | | |
| pLBJ21/antisense | 2.22 × 10⁻⁵ g | 2.39 × 10⁻⁶ |
| AtRanBP1c-2 | | |
| pLBJ21/antisense | 2.82 × 10⁻⁵ g | 2.86 × 10⁻⁶ |
| AtRanBP1c-3 | | |
| pLBJ21/antisense | 2.72 × 10⁻⁵ g | 8.37 × 10⁻⁷ |
| AtRanBP1c-5 | | |
| pLBJ21/antisense | 3.48 × 10⁻⁵ g | 2.23 × 10⁻⁶ |
| AtRanBP1b-4 | | |
| pLBJ21/antisense | 3.71 × 10⁻⁵ g | 2.04 × 10⁻⁶ |
| AtRanBP1b5 | | |

As seen in Table 7, the transgenic plants of the present invention produce seeds with a weight increased by 1.2 to 2 times or more as compared to wild type seed. In particular, Arabidopsis transformed with pLBJ21/antisense AtRanBP1b produced seeds weighting more than twice the wild type seed. In addition, the transgenic plants showed increases in leaf area, stem thickness, and the number of seeds per plant.

FIG. 12 is a photograph showing that antisense AtRanBP1b/pLBJ21 plants produce bigger seeds than wild type plants.

(vi) Comparison of the Leaf Area, Hypocotyl Length and Dry Weight of the Transgenic Plants

An Arabidopsis transformed with pLBJ21/antisense AtRanBp1b was germinated in MS medium, the characteristics of Arabidopsis in a 4-leaf-stage were observed 2 weeks after germination. The results are presented in Table 8.

TABLE 8
Length and Area of Leaf, and Length of Hypocotyl
of Transgenic Plant pLBJ21/Antisense AtRanBP1b

| | pLBJ21/antisense | |
|---|---|---|
| | AtRanBP1b | Wild type |
| Dry weight (μg) | 8.0 (±1.39) | 3.5 (±0.22) |
| Length of leaf (mm) | 4.44 (±0.13) | 2.10 (±0.05) |
| Area of leaf (mm²) | 1.96 (±0.07) | 1.4 (±0.03) |
| Length of hypocotyl (mm) | 4.95 (±0.06) | 4.15 (±0.12) |

In addition, we measured the yield of seed production, the size of the flowers, and the length of the internode. Plants were grown in soil mixed with humus and vermiculite in the ratio of 50:50 for 7 to 9 weeks in a growth chamber under growth conditions at 23° C., 60% of humidity, and 12 hours of dark and 12 hours of light. A liquid fertilizer containing 5% nitrogen, 10% aqueous phosphoric acid, and 5% aqueous potassium was provided to the plants twice a week. The characteristics of the plants were measured and the results are presented in Table 9.

TABLE 9
Phenotype of Transgenic Arabidopsis Plant
pLBJ21/Antisense AtRanBP1b

| | pLBJ21/antisense | |
|---|---|---|
| | AtRanBP1b | Wild Type |
| Number of seeds per silique | 30.24 (±1.97) | 28.43 (±1.5) |
| Number of silique per plant | 63.2 (±4.3) | 38.4 (±3.8) |
| Flower size as compared to | 156 (±5.4) | 100 |
| wild type, minor axis (%) | | |
| Flower size as compared to | 170 (±6.5) | 100 |
| wild type, major axis (%) | | |
| Diameter of 1st internode | 0.57 (±0.03) | 0.26 (±0.03) |
| (mm) | | |
| Length of 3rd internode (cm) | 1.22 (±0.1) | 0.87 (±0.08) |

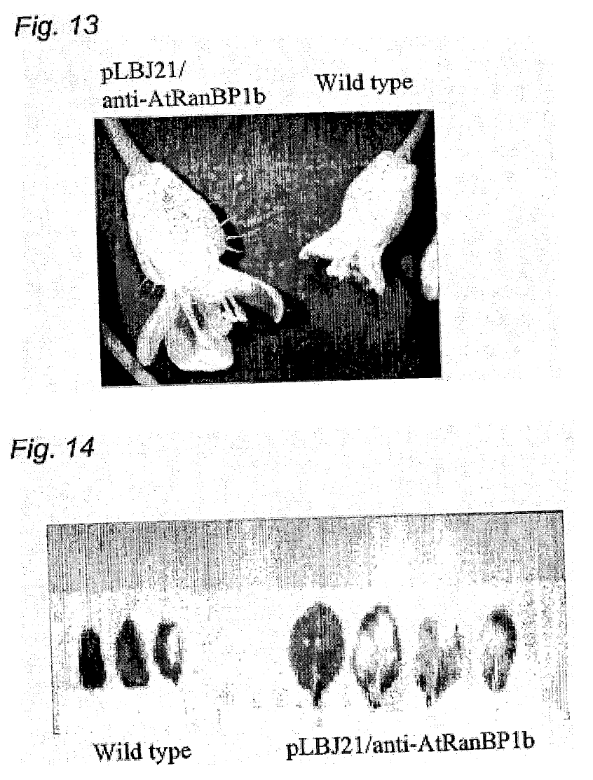

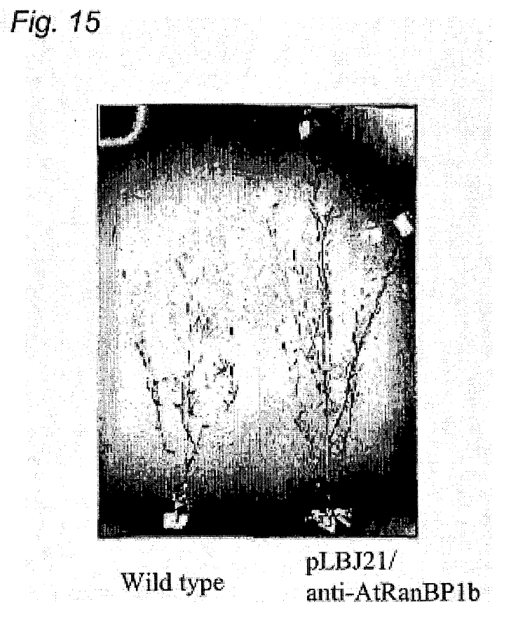

As show in FIGS. 13, 14 and 15, for Arabidopsis transformed with pLBJ21/antisense AtRanBP1b, the flower size, the leaf size, and the size of the plant itself were bigger than those of wild type plants.

Experiment 3. The Phenotype of a Transgenic Tomato

Among the transgenic tomatoes according to Examples 6 to 10, the characteristics of transgenic tomatoes transformed with pLBJ21/AtRanBP1b are presented in Table 10.

TABLE 10
Phenotype of Transgenic Tomatoes Transformed
with pLBJ21/AtRanBP1b

| | pLBJ21/AtRanBP1b | Wild type |
|---|---|---|
| Height of plant (cm) | 180 ± 6.3 | 110 ± 4.8 |
| Diameter of stem (mm) | 11.42 ± 1.3 | 7.6 ± 1.2 |
| Major axis of leaf (mm) | 116 ± 5.3 | 85 ± 3.8 |
| Dry weight of 2 cm by 2 cm | 110 ± 5 | 62 ± 1 |
| leaf sample (mg) | | |
| Thickness of leaf (mm) | 0.58 ± 0.02 | 0.27 ± 0.02 |
| Diameter of immature fruit | 39 ± 2.6 | 30 ± 1.8 |
| (mm) | | |
| Chlorophyll content per 1 g | 0.58 ± 0.08 | 0.42 ± 0.07 |
| leaf (mg/mλ solvent) | | |

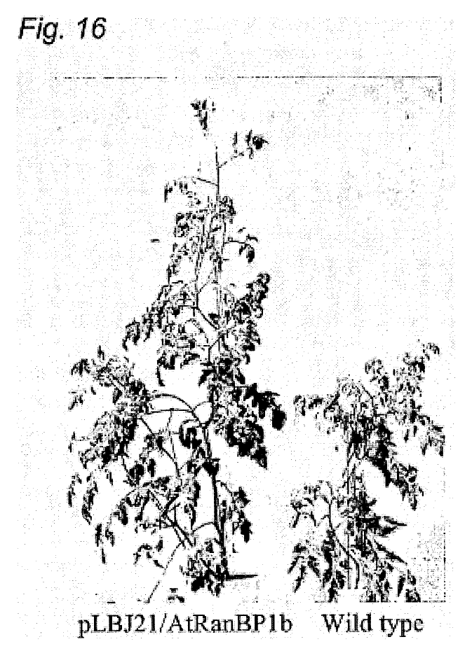

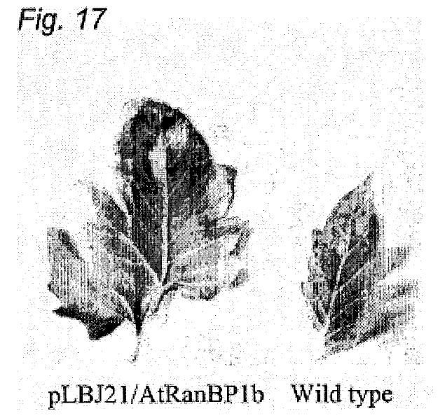

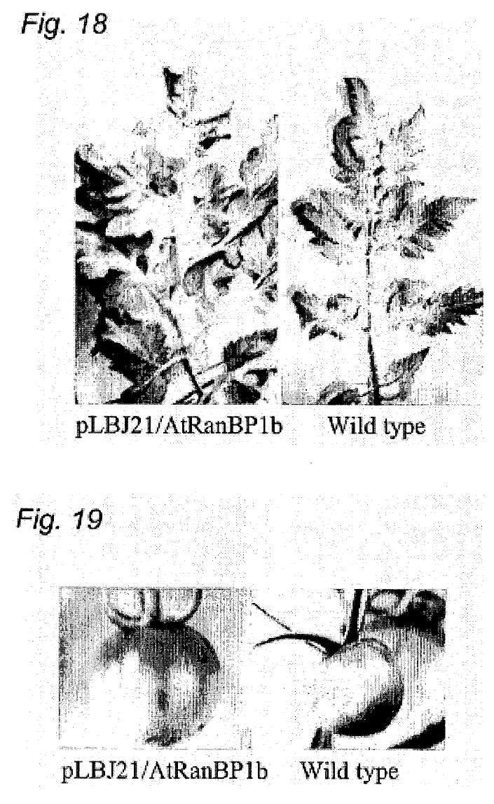

As shown in Table 10, the biomass properties of transgenic tomatoes transformed with pLBJ21/AtRanBP1b, such as plant height, the thickness, weight and size of leaf, the content of chlorophyll per weight and diameter of stem, were all significantly increased. In addition, the diameter of immature fruit increased, because the photosynthetic capacity of the leaf was increased. As shown in FIGS. 16, 17, 18 and 19, the size of adult body, the size of adult leaf, the stem thickness, and the size of immature fruit of the transgenic tomatoes were bigger than those of the comparable wild type plants.

### Example 11

### Transgenic Tomato Expressing AtRanBP1c

Tomato (Lycopersicon esculentum cv. Seokwang) was transformed by Agrobacterium-mediated tansformation. The Agrobacterium strain was Agrobacterium tumefaciens LBA4404. The vector used was the pLBJ21 binary vector, which contains the NPT II selection marker. AtRanBP1c was inserted into the vector in the sense orientation. Bacterial infection was achieved by co-culture with cotyledon segments for 2 days. Selection followed on kanamycin media. Regeneration of transformants was on media containing IAA/kinetin. Genetic transformation was confirmed by genomic PCR, RT-PCR, Southern and Western blot analysis. For Western blot analysis antibodies were raised by immunization of rabbits with AtRanBP1c expressed in E. coli using a pET vector.

Transgenic tomatoes were analyzed by PCR. The molecular phenotype (4n, chromosomal doubling) of the T0 transgenic tomato was confirmed by Giemsa staining for chromosomal counting and flow cytometric analysis of chromosomal doubling. The morphological phenotype of a T0 plant, a T0 leaf and a T0 shoot system were characterized. Genetic stability of tetraploidy in T1 plants was confirmed by flow cytometric analysis. The morphological phenotype of a T1 plant also has been characterized.

### Example 12

### Transgenic Rice

(1) Cloning of a Rice Homolog of AtRanBP1b

A rice homolog of AtRanBP1b was identified by BLAST searching a cDNA database supported by TIGR as described for the cloning of soybean homologs (see Example 18).

(2) Generation of Rice Expressing in Sense or Antisense Orientations Arabidopsis or a Rice Homolog of AtRanBP1b, OsTC84425

(2.1) Construction of Transgenic Plasmids Containing AtRanBP1a, AtRanBP1b, AtRanBP1c, Antisense AtRanBP1a, Antisense AtRanBP1b, Antisense AtRanBP1c, OsTC84425, and Antisense OsTC84425.

DNA fragments containing AtRanBP1a, AtRanBP1b, AtRanBP1c, antisense AtRanBP1a, antisense AtRanBP1b, antisense AtRanBP1c, OsTC84425, and antisense OsTC84425 were PCR amplified using sequence-specific primers for each case and were inserted into a pSB505 vector. FIG. 27 shows a schematic diagram of some transgenic constructs.

(2.2) Generation of Transgenic Rice Plants

Rice (Oryza sativa cv. Nakdong) was transformed by Agrobacterium-mediated transformation. The vector used was pSB505-BP1, which contains an actin 1 promoter and a bar marker. Constructs AtRanBP1a (sense), AtRanBP1b (sense and antisense), and AtRanBP1c (sense and antisense) were inserted into the expression vector. Bacterial infection was by co-culture of embryogenic callus for 3 days. Selection was on 2N6-CP media containing 6 mg/L of phosphinothricin. Transformants were regenerated on MSR-CP media containing 6 mg/L of phosphinothricin. MSR-CP was made up of MS media+3 mg/L PPT, 2 mg/L Kinetin, 0.5 mg/L NAA, and 4 g/L Phytagel. 2N6 was made up of N6 marco elements (50 mg/L), N6 micro element (1 mg/L), N6 vitamins (10 ml/L), FeEDTA 5, casein enzymatic hydrolysate 300 mg/L, proline 500 mg/L, glutamine 30 g/L, sucrose 30 g/L, 2,4-D 2.5 g/L, phytagel 2.5 g/L (pH 5.8). 2N6-AS (Co-cultivation media) was made up of 2N6 media with glucose 10 g/L and acetosyringone 100 uM (pH 5.2). 2N6-CP (selection media) was made by autoclaving 2N6 media, cooling to room temperature and adding hygromycin to 50 mg/L and cefotaxime to 250 mg/L (pH 5.2).

(a) Dehusked mature seeds were washed with tap water and 70% EtOH for 1 min and then sterilized with 50% Clorox by shaking for 20 min at 150 rpm and washing 3 times with sterilized water.

(b) Sterilized seeds were placed on 2N6 medium with the embryo-face up and cultured for 3 weeks at 27° C. in darkness.

(c) After 3 weeks:

Day 1—Embryogenic pieces of budding calli were selected and subcultured on new 2N6 medium at 27° C. for 4 days.

Day 2—Agrobacterium strains were streaked on AB medium with tetracycline 10 mg/L, spectinomycin 50 mg/L and incubated at 28° C. for three days to grow into a lawn.

Day 5—The Agrobacterium strain were resuspended in AAM medium containing 100 μM AS by scraping from plates with an inoculation loop. After vigorous shaking, the bacterial solution was left for 30 minutes to go into suspension (about 2 plates for 30 ml AAM solution).

OD600 should be approximately 1.8-2.0 for transformation. 20 ml of bacterial suspension (upper suspension solution) were transferred into a petri dish. The rice calli prepared as above were immersed in the bacterial suspension, gently swirled for 10 min and blotted dry on sterile filter paper. The calli were transferred to 2N6AS medium (freshly prepared) and incubated at 23-25° C. in darkness for 3 days.

Day 8—After co-cultivation, the growing calli were washed with 250 mg/L cefotaxime solution, blotted dry, placed on 2N6-CP, and cultured in darkness for 3 weeks.

(d) After 3 weeks, the actively growing calli were selected and plated on 2N6-CP for 2 weeks for secondary selection.

(e) The actively growing calli were plated on regeneration medium, MSR-CP and incubated in a 27° C. growing room with light. After 1 month, the regenerating callus was transferred to new MSR-CP medium.

(f) The generated shoots were transferred to MSO for full plant formation and rooting.

(g) After extensive rooting, the transgenic plants were transferred to soil in a greenhouse.

Genetic transformation of the rice was confirmed by PCR analysis of the bar gene from transformed rice. A total of one hundred forty eight transgenic lines were obtained from AtRanBP1a, AtRanBP1b, and AtRanBP1c transformations in sense and antisense orientations. TI seeds of AtRanBP1a were harvested and sown. The T0 molecular phenotype of tetraploidy was confirmed by flow cytometry.

### Example 13

### Transgenic Tobacco

(1) Cloning of a Tobacco Homolog of AtRanBP1b

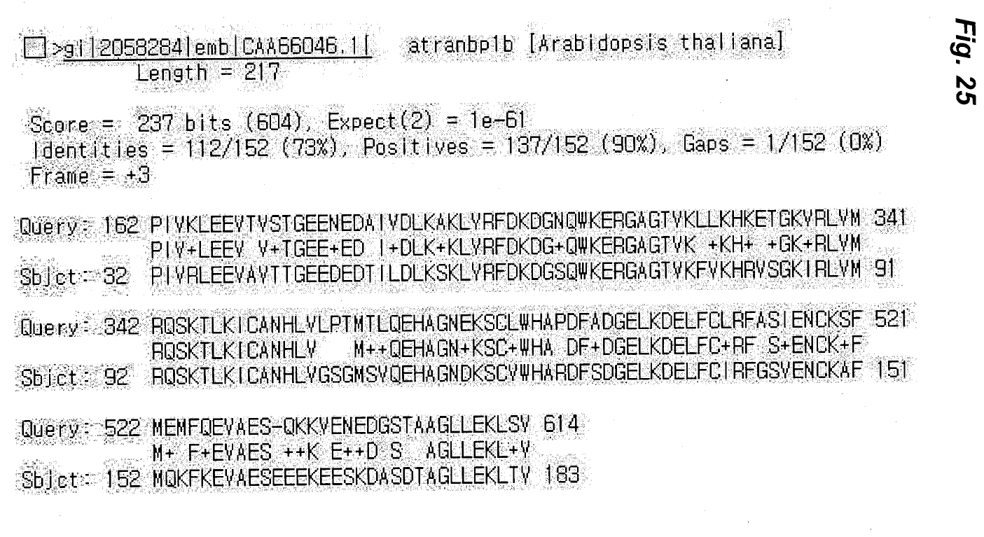

A tobacco homolog, NtRanBP1, of AtRanBP1b, NtRanBP1, was found by BLAST searching a cDNA database supported by TIGR as described for the cloning of soybean homologs or screening cDNA library of tobacco for tobacco homologs of AtRanBP1b. Screening the cDNA library with AtRanBP1b resulted in isolation of the Nt#13 clone. This is a homolog of AtRanBP1b; the two coding regions share 62% identity. FIG. 25 shows an amino acid alignment of NtRanBP1 (SEQ.ID.NO:36) and AtRanBP1b.

(2) Generation of Tobacco Expressing in Sense or Antisense Orientations Arabidopsis or Tobacco Homologs of AtRanBP1b

(2.1) Construction of the Binary Plasmids Containing AtRanBP1a, AtRanBP1b, AtRanBP1c, Antisense AtRanBP1a, Antisense AtRanBP1b, Antisense AtRanBP1c, NtRanBP1, and Antisense NtRanBP1

DNA fragments containing AtRanBP1a, AtRanBP1b, AtRanBP1c, antisense AtRanBP1a, antisense AtRanBP1b, antisense AtRanBP1c, NtRanBP1 (SEQ.ID.NO:36), and antisense NtRanBP1 (SEQ.ID.NO:37) were PCR amplified using sequence-specific primers for each nucleic acid and were inserted into the pLBJ21 binary vector.

(2.2) Generation of Transgenic Tobacco Plants

Transgenic tobacco (Nicotiana tabaccum cv. Xanthi) was prepared as follows. Transformation was mediated by Agrobacterium tumefaciens LBA4404. Some of the transformants contained AtRanBP1b in sense and antisense orientation in the pLBJ21 binary vector. Others contained the tobacco homolog Nt#13 in sense and antisense orientations in the pBIN35S vector under the CaMV35S promoter and with the NPTII selection marker. Bacterial infection was achieved by co-culture of young leaf segments for 3 days. To regenerate, callus was induced on the selection media and then transferred to the regeneration media containing BA and NAA. Transgenic lines were confirmed by Northern blot and flow cytometric analysis to determine ploidy of the plants.

(3) Results

(3.1) Seed Size Comparison Between Wild Type Tobacco and pLBJ21-Antisense AtRanBP1b Transgenic Plants

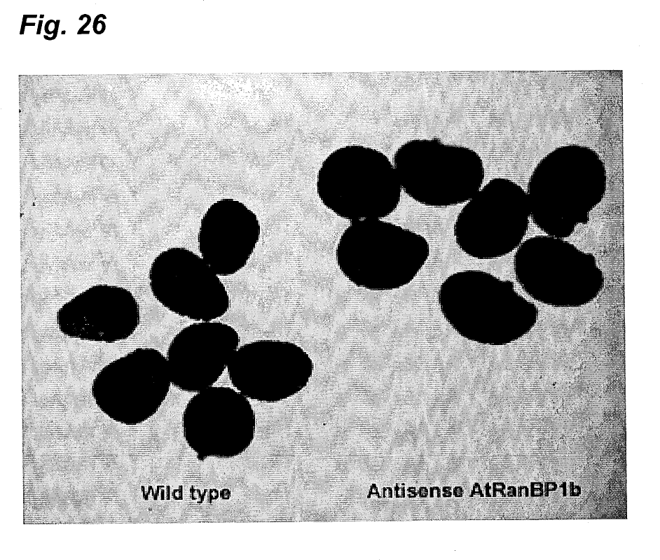

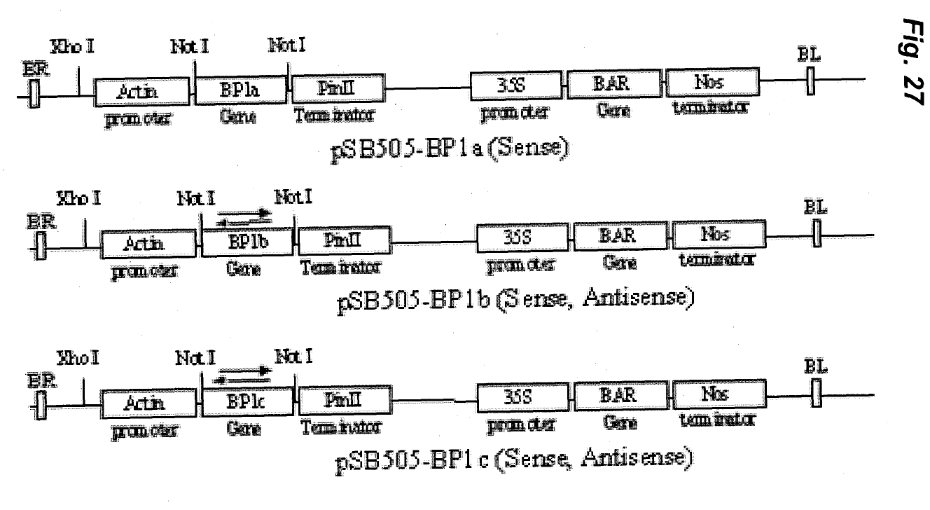

Table 11 shows the width (W) and length (L) of T1 seeds of nine independent transgenic lines measured with a ruler using a dissecting microscope. These T1 seeds were expected to segregate the transgene, and the measurements were taken from any seeds, regardless of transgene inheritance. M: mean (mm), S.D.: Standard deviation. Twenty seeds for each line were used for the measurement. As shown in the table, transgenic plants produced larger seeds than those of wild type plants. FIG. 26 shows a picture of representative seeds.

TABLE 11
Seed Size Comparison Between those from Wild Type Tobacco and
those from pLBJ21/Antisense AtRanBP1b Transgenic Plants

| | WT | | #18 | | #31 | | #28 | | #12 | | #30 | | #6 | | #35 | | | #19 | #26 | |
|---|---|---|---|---|---|---|---|---|---|---|---|---|---|---|---|---|---|---|---|---|
| | W | L | W | L | W | L | W | L | W | L | W | L | W | L | W | L | W | L | W | L |
| M | 278 | 362 | 331 | 426 | 336 | 473 | 366 | 473 | 342 | 458 | 332 | 438 | 347 | 470 | 355 | 465 | 351 | 446 | 337 | 457 |
| S.D | 26 | 21 | 21 | 31 | 18 | 27 | 44 | 30 | 19 | 31 | 23 | 30 | 23 | 30 | 33 | 14 | 30 | 27 | 20 | 27 |

(3.2) Comparison of Several Characteristics of T0 Plants of Wild Type Tobacco and pLBJ21-Antisense AtRanBP1b Transgenic Plants.

As shown in Table 12, the biomass properties of most transgenic plants increased at the T0 generation. Interestingly, the number of leaves per transgenic plant was increased without a proportional increase in height of the plant. For example for plant #28, the height increased by 12.2% as compared to that of a wild type plant and the number of leaves increased by 230%.

TABLE 12
Phenotype of pLBJ21/Antisense AtRanBP1b Transgenic
Tobacco T0 Plants

| | | | Major | | | Dry weight |
|---|---|---|---|---|---|---|
| | | Diameter | axis | Number of | Leaf | for 2 × 2 |
| | Height | of stem | of leaf | leaf per | distance | cm leaf |
| | (cm) | (mm) | (cm) | plant | (mm) | disc (μg) |
| WT | 73 | 7.5 | 20.0 | 18 | 26.6 | 32 |
| #41 | 78 | 11.0 | 17.5 | 24 | 9.1 | 43 |
| #18 | 92 | 10.4 | 17.7 | 38 | 12.6 | 33 |
| #31 | 87 | 10.2 | 17.0 | 25 | 13.9 | 40 |
| #12 | 82 | 8.9 | 15.3 | 33 | 7.4 | 31 |
| #35 | 79 | 9.4 | 17.7 | 32 | 8.1 | 38 |
| #06 | 68 | 9.8 | 18.3 | 34 | 7.5 | 34 |
| #29 | 93 | 9.0 | 18.1 | 30 | 6.7 | 33 |
| #28 | 94 | 9.4 | 14.0 | 41 | 12.1 | 31 |
| #17 | 77 | 9.7 | 21.9 | 21 | 10.4 | 35 |
| #26 | 74 | 8.5 | 15.7 | 25 | 9.8 | 42 |
| #29 | 67 | 9.7 | 17.0 | 21 | 8.0 | 33 |
| #30 | 65 | 10.9 | 17.0 | 23 | 13.0 | 40 |
| #19 | 79 | 11.4 | 18.2 | 28 | 7.5 | 43 |

Tobacco Cell Suspension Culture

A callus induction medium (CIM: 30 g/l sucrose, 4.3 g/l MS salts, 0.5 g/l MES, 1 mg/l NAA, 5 mg/l BA, 0.8% plantagar (pH 5.7)) plate was prepared and tobacco leaves were detached from wild type tobacco, mixo, 4n sense, 4n antisense transformants. The remaining tissue was cut into 1 cm lengths and placed cut side down in the agar. The explants were incubated and watched daily for callus formation. Once sufficient callus growth took place, it was carefully removed from the explant and transferred to fresh medium. The callus line was maintained by subculturing every 2-4 weeks (25° C., 16:8 L/D).

The resulting callus was removed from the CIM Petri dish and transferred to a sterilized Petri dish. Callus blocks were trimmed with a scalpel and only the young, actively growing callus was used for the inoculum. Each flask had 100 ml MS0+1 mg/l 2,4-D liquid medium, and received an inoculum of about 500-750 mg of callus in order to initiate the culture. The inoculated flasks were placed in a shaker at a speed of 100 rpm. The shaker with the flasks was placed in an air-conditioned enclosure maintained at 25° C. in the dark. After 7-10 days, the culture was filtered through a sieve in order to remove the residual inoculum and larger clumps of cells. The culture was maintained by subculturing after 10 days.

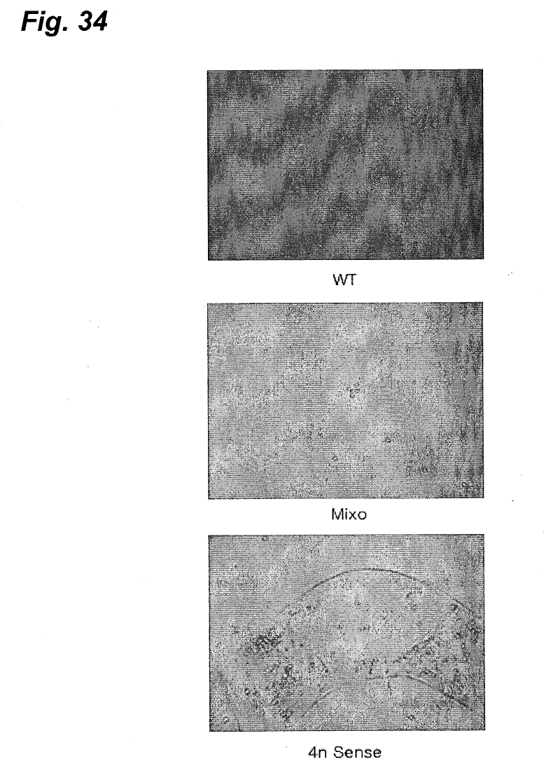

As shown in FIG. 34, the cells of Mixo and 4n Sense are much larger than those of Wild type.

### Example 14

### Generation of Transgenic Cotton

(1) Cloning of a Cotton Homolog of AtRanBP1b

Cotton homologs of AtRanBP1b were cloned either by screening a cDNA library of cotton expanding leaves or by BLAST searching a cotton cDNA database supported by TIGR as described for the cloning of soybean homologs (see Example 18).

(1.1) Cloning of GhRanBP1-1 and GhRanBP1-2

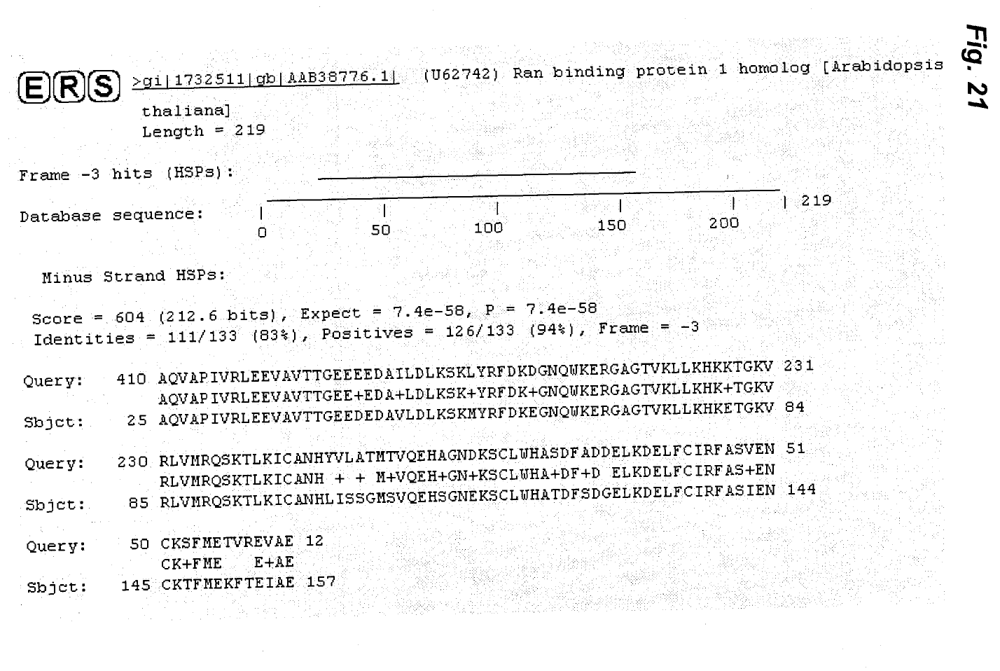

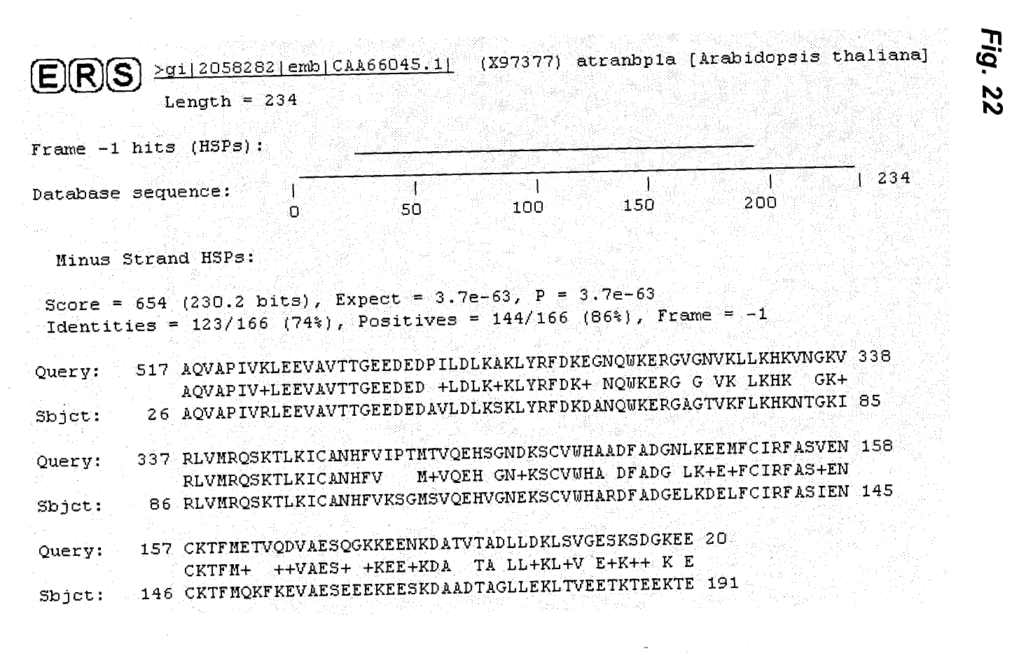

DNA encoding cotton Ran-binding proteins was PCR amplified from an expanding leaf cDNA library using the 5′ primer of cotton RanBP1a,c-S 5′-GCTCAAGTTGCCCCTATCGTC-3′, cotton RanBP1b-S 5′-TGCTCAAGTTCCTATCGTCAAG-3′ and the 3′ primer cotton RanBP1a,c-AS 5′-ATTCAGCAACTTCTCGGACCG-3′, cotton RanBP1b-AS 5′-CGACCGTATCTTCTTCTTTACCGTC-3′ at an annealing temperature of 57° C. The PCR products were cloned in the plasmid vector pGEM-T Easy (Promega) and the DNA sequence of the inserts was analysed. FIG. 21 shows an alignment of the amino acid sequence of AtRanBP1 and GhRanBP1-1 (SEQ.ID.NO:20), and FIG. 22 shows an alignment of AtRanBp1 and GhRanBP1-2 (SEQ.ID.NO:22).

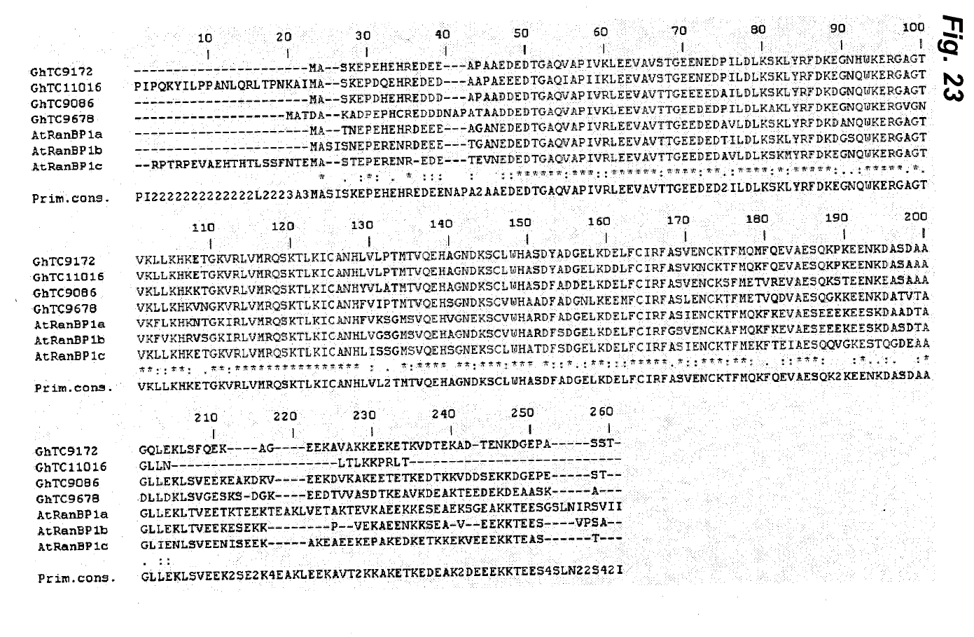

(1.2) Cloning of GhTC9172, GhTc11016, GhTC9086, and GhTC9678 GhTC9172, GhTc11016, GhTC9086, and GhTC9678 are cotton homologs of AtRanBP1b which were cloned by BLAST searching a cotton cDNA database supported by TIGR as described for the cloning of soybean homologs. FIG. 23 shows an amino acid alignment of the AtRanBP1b homolog proteins.

(2) Generation of Cotton Expressing DNA in Sense or Antisense Orientations Encoding Arabidopsis or Cotton Homologs of AtRanBP1b

(2.1) Construction of the Binary Plasmid Containing AtRanBP1a, AtRanBP1b, AtRanBP1c, Antisense AtRanBP1a, Antisense AtRanBP1b, Antisense AtRanBP1c, GhRanBP1-1 (SEQ.ID.NO:20), Antisense GhRanBP1-1 (SEQ.ID.NO:21), GhRanBP1-2 (SEQ.ID.NO:22), Antisense GhRanBP1-2 (SEQ.ID.NO:23), GhTC9172 (SEQ.ID.NO:24), Antisense GhTC9172 (SEQ.ID.NO:25), GhTC11016 (SEQ.ID.NO:26), Antisense GhTC11016 (SEQ.ID.NO:27), GhTC9086 (SEQ.ID.NO:28), Antisense GhTC9086 (SEQ.ID.NO:29), GhTC9678 (SEQ.ID.NO:30), and Antisense GhTC9678 (SEQ.ID.NO:31)

DNA fragments containing AtRanBP1a, AtRanBP1b, AtRanBP1c, antisense AtRanBP1a, antisense AtRanBP1b, antisense AtRanBP1c, GhRanBP1-1 (SEQ.ID.NO:20), antisense GhRanBP1-1 (SEQ.ID.NO:21), GhRanBP1-2 (SEQ.ID.NO:22), antisense GhRanBP1-2 (SEQ.ID.NO:23), GhTC9172 (SEQ.ID.NO:24), antisense GhTC9172 (SEQ.ID.NO:25), GhTC11016 (SEQ.ID.NO:26), antisense GhTC11016 (SEQ.ID.NO:27), GhTC9086 (SEQ.ID.NO:28), antisense GhTC9086 (SEQ.ID.NO:29), GhTC9678 (SEQ.ID.NO:30), or antisense GhTC9678 (SEQ.ID.NO:31) were PCR amplified using sequence-specific primers for each nucleic acid and were inserted into the pLBJ21 binary vector.

(2.2) Generation of Cotton Transgenic Plants

Cotton (Gossynipium hirsutum L. cv Suwon3 and cv. Suwon 8) was subjected to transformation mediated by Agrobacterium strain LBA4404. Genes encoding the following proteins were inserted in the indicated orientation in a pLBJ21 binary vector: (1) sense At RanBP1b #5, AtRanBP1b #6, and AtRanBP1b #8, and (2) antisense AtRanBP AS 1b-3, AtRanBP AS 1b-4, AtRanBP AS 1c-1, and AtRanBP AS 1c-2, AtRanBP AS 1c-3. Seeds were surface-sterilized and seed coats were removed. The seeds without coats were further sterilized with 30% Clorox in 0.025% Triton-X-100 and cultured on MSO plate (30 g/l sucrose, 4.3 g/l MS salts, 0.5 g/l MES, 8 g/l Phytoagar, pH 5.7) at 25° C. with 16:8 light/day cycle. Bacterial infection and regeneration was by bisection of shoot apex of the 12-day old sterile seedlings. The seedling shoot was embedded in the stem between the cotyledons. The cotyledon was removed by pushing down on it until it snapped off. This exposed the shoot apex. The shoot was removed from the seedling and cultured in MS+Kin medium (MSO+0.1 mg/l kinetin) in the dark, 25° C. for 3 days. The shoot apex was then sonicated for several seconds or picking pins to inoculate Agrobacterium containing the fusion constructs described above. After 3 days of co-cultivation on MS+Kin plates in the dark at 25° C., shoot apexes were transferred to MS+C medium (MSO+500 mg/l Carbenicilin) for re-culture. After 7 days of the culture, transformants were selected on MS+Kin+C+Kan medium (MSO+0.1 mg/l kinetin+500 mg/l carbenicilin+10 mg/l Geneticin). After 2-4 weeks selection in Magenta box, surviving shoots were transferred to MSO medium for rooting for 2-4 weeks. Transformant lines grew on regeneration media.

### Example 15

### Transgenic Pepper

Capsicum annuum L. cvs. Hungnong, Keumtap and Nokwang were subject to Agrobacterium-mediated transformation. The genes encoding AtRanBP1b (sense and antisense) and AtRanBP1c (sense and antisense) were inserted using the vectors pLBJ21 (sense) and pBIN35S (antisense), each of which has a CaMV35S promoter and a kanamycin selection marker. Bacterial infection was by co-culture of hypocotyls and cotyledon segments for 2 days. Selection was on MS media containing NAA/BA and kanamycin. The transformants generated shoots.

### Example 16

### Generation of Transgenic Potato

(1) Cloning of Potato Homologs of AtRanBP1b

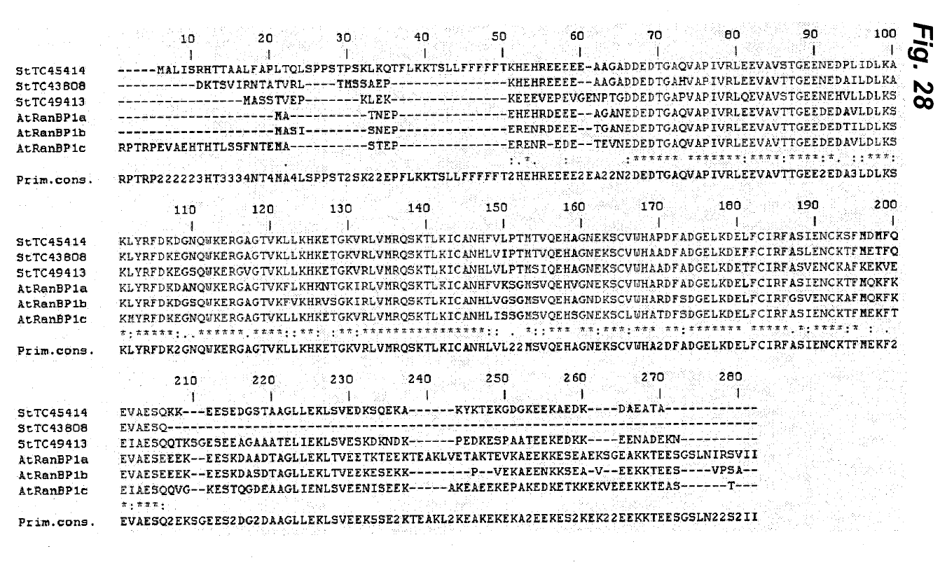

Potato homologs of AtRanBP1b, StTC45414 and StTC43808, were found by BLAST searching a maize cDNA database supported by TIGR as described for the cloning of soybean homologs. FIG. 28 shows an alignment of the amino acid sequences.

(2) Generation of Potato Expressing in Sense or Antisense Orientations Arabidopsis or Potato Homologs of AtRanBP1b, StTC45414, StTC43808

(2.1) Construction of a Transformation Plasmid Containing AtRanBP1a, AtRanBP1b, AtRanBP1c, Antisense AtRanBP1a, Antisense AtRanBP1b, Antisense AtRanBP1c, StTC45414, Antisense StTC45414, StTC43808, or Antisense StTC43808

DNA fragments encoding AtRanBP1a, AtRanBP1b, AtRanBP1c, antisense AtRanBP1a, antisense AtRanBP1b, antisense AtRanBP1c, StTC45414 (SEQ.ID.NO:38), antisense StTC45414 (SEQ.ID.NO:39), StTC43808 (SEQ.ID.NO:40), or antisense StTC43808 (SEQ.ID.NO:41) were PCR amplified using sequence-specific primers and inserted into a pLBJ21 vector for sense strand expression or a pBIN35S vector for antisense expression.

(2.2) Generation of Transgenic Potato Plants

Expression constructs described above were inoculated into a cotyledon or an embryo of Solanum tuberosum L. cv. Su-mi using Agrobacterium-mediated transformation as described for soybean (see Example 18).

### Example 17

### Generation of Transgenic Maize

(1) Cloning of Maize Homologs of AtRanBP1b

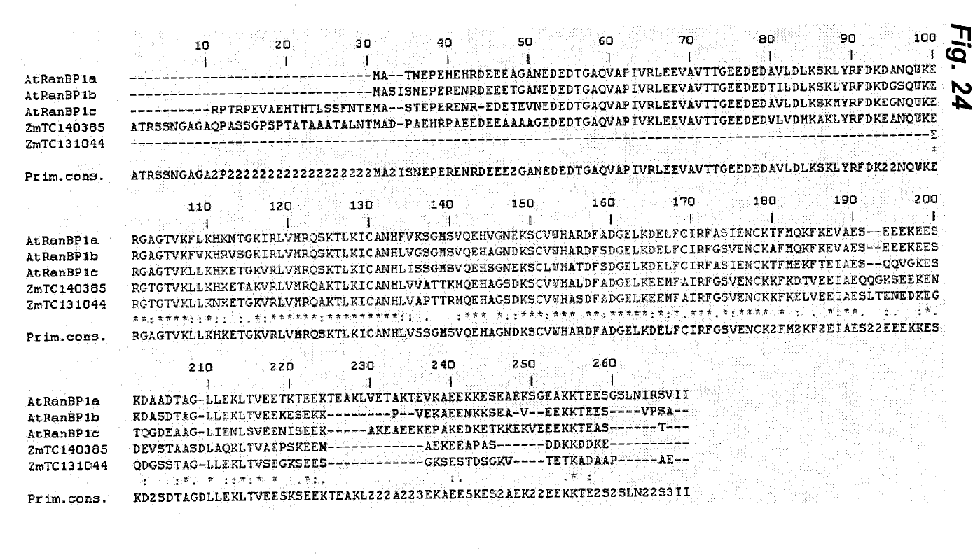

Maize homologs of AtRanBP1b, ZmTC140385 and ZmTC131044, were found by BLAST searching a maize cDNA database supported by TIGR as described for the cloning of soybean homologs (see Example 18). FIG. 24 shows an alignment of the amino acid sequences.

(2) Generation of Maize Expressing in Sense or Antisense Orientations Arabidopsis or Maize Homologs of AtRanBP1b, ZmTC140385 and ZmTC131044.

(2.1) Construction of Transformation Plasmids Containing AtRanBP1a, AtRanBP1b, AtRanBP1c, Antisense AtRanBP1a, Antisense AtRanBP1b, Antisense AtRanBP1c, ZmTC140385, Antisense ZmTC140385, ZmTC131044, and Antisense ZmTC131044

DNA fragments encoding AtRanBP1a, AtRanBP1b, AtRanBP1c, antisense AtRanBP1a, antisense AtRanBP1b, antisense AtRanBP1c, ZmTC140385 (SEQ.ID.NO:32), antisense ZmTC140385 (SEQ.ID.NO:33), ZmTC131044 (SEQ.ID.NO:34), and antisense ZmTC131044 (SEQ.ID.NO:35) were PCR amplified using sequence-specific primers for each nucleic acid and were inserted into the pAHC17 vector (Christensen and Quail, Transgenic Res. 5:213-218 (1996)).

(2.2) Generation of Transgenic Maize Plants

The expression constructs described above were co-bombarded with a herbicide selection plasmid (pBARGUS) into embryogenic Hill calli using biolistic delivery. pBARGUS is described at the Plant Transformation Facility, Iowa State University website: <ww.agron.iastate.edu/ptf/web/mainframe.htm>. The transformed calli were checked by RNA gel blot analysis for transgene expression and tested for resistance to 3 mg/mL bialaphos, the active ingredient in Basta® herbicide (Spencer et al., 1990). The doubly positive calli were regenerated into T0 plants.

Alternatively, maize (Zea mays) was subjected to Agrobacterium-mediated transformation using the Agrobacterium tumefaciens LBA4404 strain. DNA encoding AtRanBP1b was inserted in sense and antisense orientations in the pLBJ21 binary vector, with a CaMV35S Promoter and a NPT 11 selection marker. Immature zygotic embryos were collected, sterilized and cultured on hormone-free solid N6 medium in the dark for 2-3 days. Embryos were inoculated with Agrobacterium for 30 min followed by co-cultivation in N6 inoculation medium containing hormones and silver nitrate for 3 days. Embryos were then transferred to B5 selection medium. Regenerants from embryogenic callus were grown on regeneration medium. Genetic transformation was confirmed by genomic PCR, RT-PCR, Southern and Northern blot analysis.

### Example 18

### Generation of Transgenic Soybean

(1) Transformation of Soybean

To produce transgenic soybean plants with RanBP genes, binary vectors were constructed with hpt and NTPII selection markers. RanBP genes were introduced into soybean by cotyledon node and SAAT transformation methods.

(2) Cloning of the Soybean Homolog of AtRanBP1b

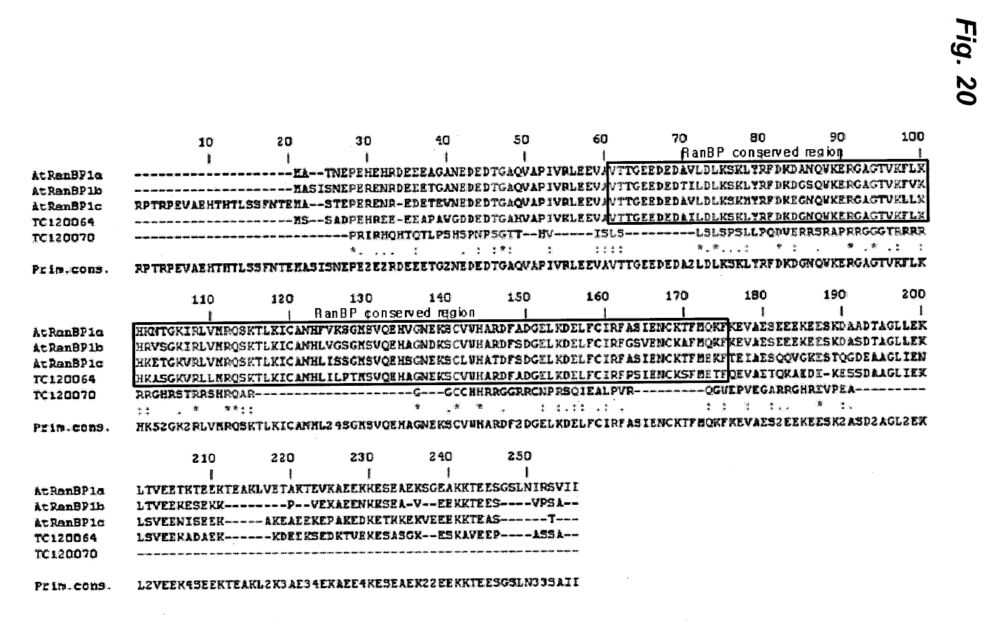

The nucleotide sequence of the previously described AtRanBP1b was used for BLAST searches against TIGR database <http://tigrblast.tigr.org/tgi/>. TC120064 and TC120070 (GmGI) were identified and found to encode a protein very similar to the Arabidopsis Ran-binding protein AtRanBP1b, and to contain the conserved Ran-binding domain. FIG. 20 shows amino acid alignment data among them.

(3) Generation of Soybeans Expressing in Sense or Antisense Orientation DNA Encoding AtRanBP1b or Soybean Homologs TC120064 and TC120070.

(3.1) Construction of the Binary Plasmid Containing DNA Encoding AtRanBP1a, AtRanBP1b, AtRanBP1c, Antisense AtRanBP1a, Antisense AtRanBP1b, Antisense AtRanBP1c, TC120064, Antisense TC120064, TC120070, and Antisense TC120070.

DNA fragments encoding AtRanBP1a, AtRanBP1b, AtRanBP1c, antisense AtRanBP1a, antisense AtRanBP1b, antisense AtRanBP1c, TC120064 (SEQ.ID.NO:16), antisense TC120064 (SEQ.ID.NO:17), TC120070 (SEQ.ID.NO:18), and antisense TC120070 (SEQ.ID.NO:19) were PCR amplified using sequence-specific primers for each nucleic acid and were inserted into a pCAMBIA1300 binary vector.

(3.2) Generation of Transgenic Soybean Plants

The fusion binary plasmids were transformed into the Agrobacterium tumefaciens strain LBA4404 by electroporation. Transformed A. tumefaciens containing the pCAMBIA1300 fusion constructs were cultured overnight on a shaker at 29° C./200 rpm in 20 ml of liquid YEP medium containing 50 μg/ml kanamycin. The A. tumefacines suspension was pelleted by low-speed centrifugation using a table-top centrifuge and then resuspended to an OD600 of 1.0 or 0.2 in liquid D40 medium (MS salts, B5 vitamins, 40 mg/l 2,4-D, 6% sucrose, pH 7.0) with 100 μM acetosyringone being added immediately before cotyledon infection.

Seeds were soaked in ddH₂O at RT for 1 hour. After soaking in water, the seeds were sterilized in 70% EtOH for 2 min, and then 30% commercial bleach for 5 min. Sterilized seeds were washed several times with sterilized water in a clean room. After drying, the seeds were germinated in B5 medium (Gamborg B5 medium, B5 vitamin, 20 mM MES, 3% sucrose, pH 5.7, 0.8% plant agar) for 3-5 days.

To prepare cotyledonary nodes to be inoculated by the above constructs, the seedlings were harvested and the seed coats removed. There was a 3-4 cm radicle, and a developing shoot axis. The radicle was cut off, leaving an approximately 3 mm hypocotyl. With a scalpel, the seedling was bisected directly through the cotyledonary node. The seedlings were inoculated for 30 min. Following cocultivation with the A. tumefacines for 3 days at 24° C., the seedlings were placed on shoot induction medium. The shoot induction medium included full-strength B5/B5 medium supplemented with 1.7 mg/L BAP, pH 5.8 and 3% (w/v) sucrose, 5 μM benzyladenine, 10 μM Indole butyric acid, 50 mg/L hygromycin, 100 mg/L carbenicillin, 30 mg/L timentin, 100 mg/L vancomycin. Prior to rooting, shoots that were too small were transferred to B5 elongation medium that included B5/B5 medium supplemented with 20 μM MES, 1 mM indole butyric acid, 3% sucrose, 1 mg/L GA3 pH 5.7, 0.8% plant agar, until they reached a length of at least ½ cm. When the shoots were longer than ½ cm they were transferred to hygromycin-containing media, which is a more stringent selection for true transformants. Whereas non-transformed shoots may be recovered from cotyledonary nodes on hygromycin, they will not root on B5 rooting medium containing B5/B5 medium supplemented with 20 μM MES, 10 mM indole butyric acid, 3% sucrose, pH 5.7, 0.8% plant agar, 250 mg/L hygromycin.

In some cases trangenic plants were generated by sonication-assisted Agrobacterium-mediated transformation (SAAT). This method is a simple, low cost method that minimally enhances the efficiency of Agrobacterium-mediated transformation of or non-susceptible plant species. The strength of this method is that the cavitation caused by sonication results in thousands of microwounds on and below the surface of the plant tissue. This wounding pattern permits Agrobacterium to travel deeper into and more completely throughout the tissue than does conventional microscopic wounding, increasing the probability of infecting the plant cells.

Seed sterilization and cotyledon preparation were performed as described above. Cotyledons were excised from the germinated soybean seeds and placed on D40 medium, 0.8% plant agar until enough cotyledons had been harvested for SAAT treatment. Approximately 10 cotyledons were placed in a 1.5 ml microcentrifuge tube along with 0.5 ml of the Agrobacterium suspension. Tissue was SAAT-treated for two seconds. Approximately five minutes after SAAT-treatment, excess Agrobacterium was removed by blotting on a sterile filter paper. Cotyledons were then placed on D40 medium, 100 μM acetosyringone, 0.8% plant agar and co-cultivated with the Agrobacteria for three days. Cotyledons were transferred to D40 medium, 500 mg/l cefotaxime, 0.8% plant agar and shoots were induced as described above.

Soybean (Glycine max cv. Jangyeop) also were subjected to Agrobacterium-mediated transformation using Agrobacterium tumefaciens LBA4404 strain into wich AtRanBP1b in sense and antisense orientations had been inserted using the pLBJ21 binary vector with a CaMV35S promoter and a NPT II selection marker. Seeds were sterilized and germinated on hormone-free solid B5 medium in dark condition. The cotyledonary node region was inoculated with Agrobacterium for 30 min, followed by co-cultivation on B5 inoculation medium for 3 days. Selection was on B5 selection medium including hormones and antibiotics, followed by shoot induction in B5 elongation medium containing IAA, GA3, and root induction in B5 rooting medium. Genetic transformation was confirmed by genomic PCR, RT-PCR, Southern and Northern blot analysis.

### Example 19

### Generation of Transgenic Wheat

(1) Cloning of Wheat Homologs of AtRanBP1b

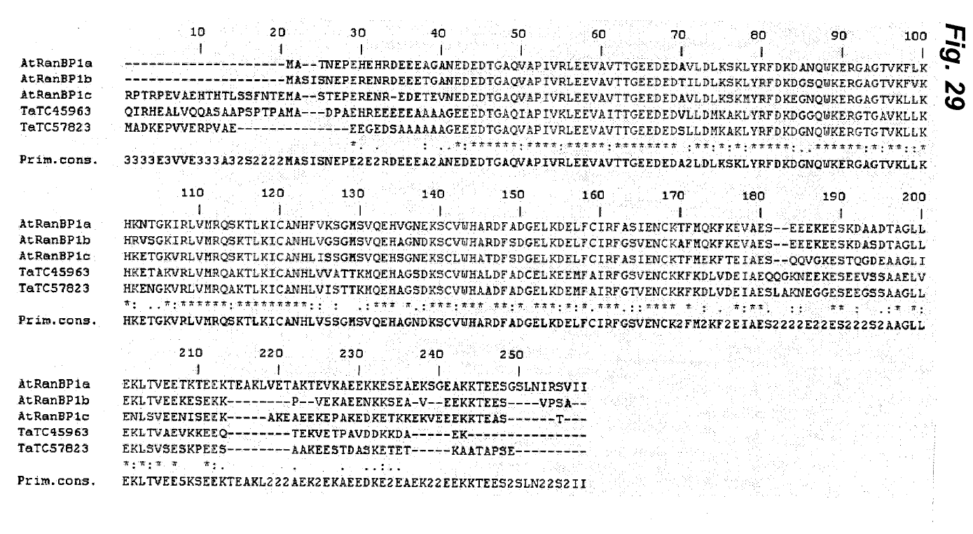

Wheat homologs of AtRanBP1b were found by BLAST searching a maize cDNA database supported by TIGR as described for the cloning of soybean homologs (see Example 18). FIG. 29 shows their amino acid sequence alignment.

(2) Generation of Wheat Expressing in Sense or Antisense Orientation Arabidopsis or Wheat Homologs of AtRanBP1b

(2.1) Construction of the Fusion Plasmids Containing AtRanBP1a, AtRanBP1b, AtRanBP1c, Antisense AtRanBP1a, Antisense AtRanBP1b, Antisense AtRanBP1c, TaTC45963, Antisense TaTC45963, TaTC57823, Antisense TaTC57823

DNA fragments containing AtRanBP1a, AtRanBP1b, AtRanBP1c, antisense AtRanBP1a, antisense AtRanBP1b, antisense AtRanBP1c, TaTC45963 (SEQ.ID.NO:42), antisense TaTC45963 (SEQ.ID.NO:43), TaTC57823 (SEQ.ID.NO:44), and antisense TaTC57823 (SEQ.ID.NO:45) were PCR amplified using sequence-specific primers and inserted into a pAHC17 vector (Christensen and Quail, Transgenic Res. 5:213-218 (1996).

(2.2) Generation of Transgenic Wheat Plants

Expression constructs described above were transformed into spring wheat variety Scamp using Agrobacterium-mediated transformation as detailed below.

(a) Bacterial Strain, Culture Conditions

Plants of the spring wheat variety Scamp were grown in the greenhouse at 18° C. day and 15° C. night temperatures, with a photoperiod of 16 h. The 14-day-old embryos were isolated and sterilized in 15% Domestos solution (Diversey Lever) for 20 min, rinsed three times with sterile water, and then subcultivated on MS medium at 26° C. in the dark overnight. Agrobacterium strain, AGL1, containing one of the above constructs was grown in MG/L medium containing 1 mg/L biotin, 100 mg/L kanamycin and 200 mg/L carbenicillin at 26-27° C., in the dark, with shaking at 250 rpm. Cells were resuspended in L7P4-V medium containing 10 g/L glucose to give an OD600 of up to 2.0. Acetosyringone was added just prior to inoculation, to a final concentration of 200 μM.

(b) Inoculation, Co-Cultivation and Regeneration

Explants were transferred to inoculum medium, ensuring that all explants were completely submerged. Inoculation was carried out in the dark at 26° C., for up to 3 h. Explants were dried on sterile filter paper and then plated onto callus induction medium containing 10 g/L glucose and acetosyringone at 200 μM. Co-cultivation with the Agrobacteria was carried out in the dark at 26° C. for 2-3 days. After co-cultivation, a proportion of the explants were transferred to generation medium with or without selection and induction medium to form shoots and roots.

Alternatively, wheat (spring wheat variety Scamp) was subjected to sonication-assisted, Agrobacterium-mediated transformation. Fourteen-day-old embryos were isolated, sterilized, and cultured on MS medium at 26° C. in the dark overnight. Sonication treatment was for 6 seconds at a 40 kHZ or higher frequency. Embryos were incubated on MS-induction medium (2, 4-D, maltose, gelrite) for selection and regeneration. Genetic transformation was confirmed by genomic PCR, RT-PCR, Southern and Northern blot analysis.

### Example 20

### Generation of Transgenic Alfalfa

(1) Cloning of Alfalfa Homolog of AtRanBP1b

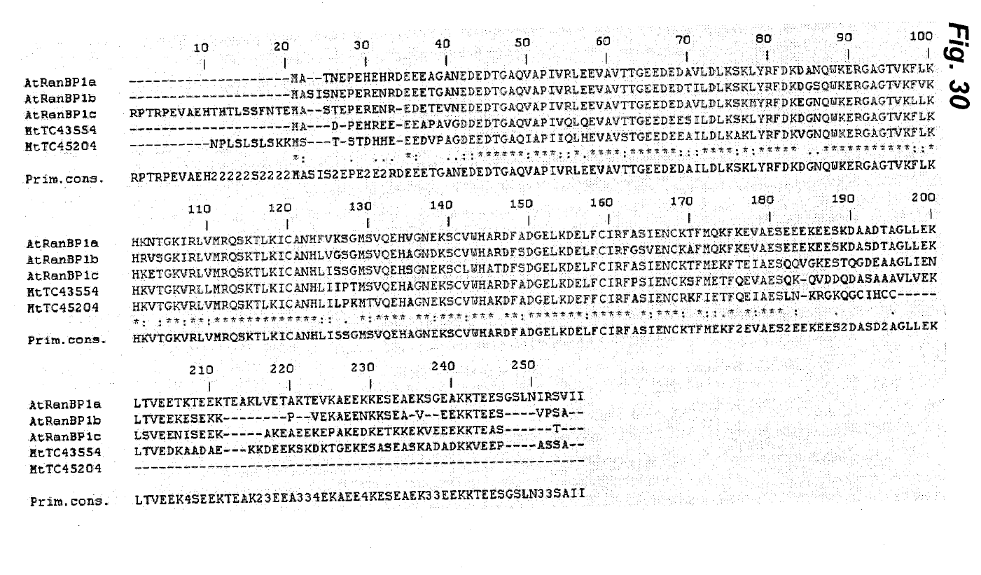

Alfalfa Homologs of AtRanBP1b were found by BLAST searching a cDNA database supported by TIGR as described for the cloning of soybean homologs. FIG. 30 shows their amino acid sequence alignment.

(2) Generation of Alfalfa Expressing in Sense or Antisense Orientations Arabidopsis or Alfalfa Homologs of AtRanBP1b

(2.1) Construction of the Fusion Plasmids Containing AtRanBP1a, AtRanBP1b, AtRanBP1c, Antisense AtRanBP1a, Antisense AtRanBP1b, Antisense AtRanBP1c, MtTC43554, Antisense MtTC43554, MtTC45204, Antisense MtTC45204

DNA fragments containing AtRanBP1a, AtRanBP1b, AtRanBP1c, antisense AtRanBP1a, antisense AtRanBP1b, antisense AtRanBP1c, MtTC43554 (SEQ.ID.NO:46), antisense MtTC43554 (SEQ.ID.NO:47), MtTC45204 (SEQ.ID.NO:48), and antisense MtTC45204 (SEQ.ID.NO:49) were PCR amplified using sequence-specific primers for each case and were inserted into and pLBJ21 vector.

(2.2) Generation of Transgenic Alfalfa Plants

Expression constructs described above were transformed into Medicago truncatula using Agrobacterium-mediated transformation into flower meristem by vacumn infiltration as described below and by Anthony et al., The Plant Journal 22:531-541 (2000) (See also Mark et al., J. Exp. Bot. 51:29-39 (2000).

(a) Sterilization and vernalization of M. trunatula seeds

Seeds were soaked in concentrated sulphuric acid for approximately 10 min, rinsed three times with sterile distilled water and then sterillized in 30% chlorox, 0.1% Tween 20 for 5 min with gentle agitation. The seeds were then washed three times in sterile distilled water and spread on 0.8% water agar in petri plates, approximately 25 seeds per plate. The plates were wrapped with parafilm and aluminum foil. The seeds were then vernalized by incubating at 4° C. for 2 weeks.

(b) Preparation of Agrobacterium for Flower Infiltration

Agrobacterium containing one of the above constructs was grown at 28° C. in YEP medium (Peptone 10 g, yeast extract 10 g, NaCl 5 g) containing the appropriate antibiotics to OD600 of 1.6-1.8. The cells were collected by centrifugation and resuspended in flower infiltration media (0.5×MS salts, 1× Gamborg's vitamins, 0.04 μM BAP, 0.02% Silwet77), pH 5.7.

(c) Transformation of M. Turncatula by Vacuum Infiltration into Flowering Plants

Plants were grown until the plants had small flower buds and a few opened flowers. This occured approximately 4 weeks after planting. The plants were watered heavily on the day before infiltration took place. To infiltrate the plants, the pots were inverted and the above-ground portion of the plant submerged in a container filled with a suspension of Agrobacterium in flower infiltration medium. The pot and tray were placed in a vacuum chamber and a vacuum drawn to 25 inches Hg and held for 3 min. The vacuum was released very rapidly and the procedure repeated once. The pots were removed from the Agrobacterium-containing medium and placed on their sides in a tray to prevent the Agrobacterium from entering the soil. The tray and pots were transferred to a growth chamber set at 18° C., 95% humidity, 16 h days with cool, white lights. The plants were incubated in the chamber for a week. After 2-3 days the pots were placed in an upright position again. The pots were not watered during this time. After a week, the infiltration process was repeated and the plants returned to the growth chamber for a week. In some cases the plants required water during the second week and this was applied carefully to the bottom of the pots. After the second week the plants were returned to normal growing conditions and allowed to set seed. Transformants were selected in the subsequent generation.

### Example 21

### Generation of Transgenic Sorghum

(1) Cloning of Sorghum Homolog of AtRanBP1b

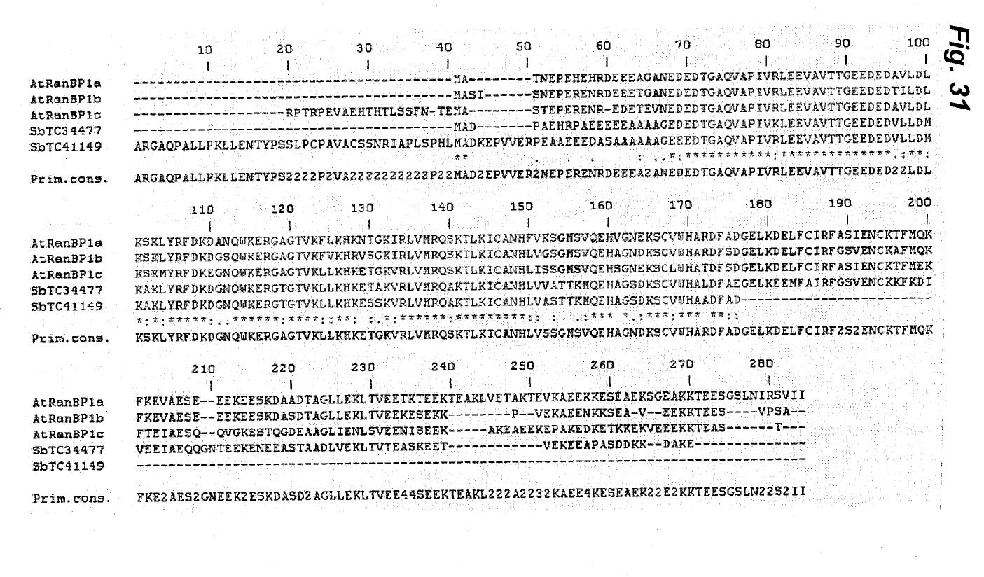

Sorghum homologs of AtRanBP1b were found by BLAST searching a cDNA database supported by TIGR as described for the cloning of soybean homologs (see Example 18). FIG. 31 shows their amino acid sequence alignment.

(2) Generation of Sorghum Expressing in Sense or Antisense Orientations Arabidopsis or Sorghum Homologs of AtRanBP1b

(2.1) Construction of the Fusion Plasmids Containing AtRanBP1a, AtRanBP1b, AtRanBP1c, Antisense AtRanBP1a, Antisense AtRanBP1b, Antisense AtRanBP1c, SbTC34477, Antisense SbTC34477, SbTC41149, Antisense SbTC41149

DNA fragments containing AtRanBP1a, AtRanBP1b, AtRanBP1c, antisense AtRanBP1a, antisense AtRanBP1b, antisense AtRanBP1c, SbTC34477 (SEQ.ID.NO:50), antisense SbTC34477 (SEQ.ID.NO:51), SbTC41149 (SEQ.ID.NO:52), and antisense SbTC41149 (SEQ.ID.NO:53) were PCR amplified using sequence-specific primers for each case and were inserted into the pSB505 vector as described for rice transformation above.

(2.2) Generation of Transgenic Sorghum Plants

The expression constructs described above were transformed into embryo of P89012 using Agrobacterium-mediated transformation as described below and by Zhao et al., Plant Mol Biol 44:789-798 (2000).

(a) Sterilization

Sorghum plants were grown in a greenhouse. Immature panicles were harvested 9-12 days after pollination. The kernels were sterilized with 50% bleach and 0.1% Tween-20 for 30 min with vacuum. The kernels were rinsed with sterile water three times. Immature zygotic embryos (1.0-1.5 cm in length) were isolated from the kernels.

(b) Agroinfection

The freshly isolated embryos were immersed in an Agrobacterium suspension at either 1×10⁹ cgu/ml (OD=0.7 at 550 nm) or 0.5×10⁹ cfu/ml in PHI-I medium for 5 min for the agroinfection process. For all subsequent culture steps, the embryos were cultured with the scutellar side up and facing away from the medium.

(c) Co-Cultivation

The immature embryos were cultured on PHI-T medium in the dark at 25° C. for 3 or 7 days for the co-cultivation step.

(d) Resting

For the resting step the embryos were cultured on PHI-T medium (without acetosyringon) plus 100 mg/l carbenicillin for 0 or 4 days. The resting step was carried out at 28° C. in the dark.

(e) Selection

The embryos were moved to PHI-U medium for 2 weeks to initiate the selection step then moved to PHI-V medium for the remainder of the selection process. The selection step was carried out at 28° C. in the dark. The subculture interval was typically 2 weeks; however, if the sorghum tissue produced more phenolic pigment, the subculture interval was reduced to 5-7 days.

(f) Regeneration

For plant regeneration, the herbicide-resistant callus was maintained for 2-3 weeks to increase the amount of putative transformed callus and to develop somatic embryos, and then cultured on PHI-X medium for 2-3 weeks to develop shoots. When shoots started to appear, the cultures were moved to a room under conditions of 16 h light (270 μE m⁻² s⁻¹) and 8 h dark at 25° C. Shoots (about 3-5 cm tall) were moved to plastic boxes (10 cm×9 cm×10 cm) containing or PHI-Z medium if the shoots had good roots; or PHI-FA medium if shoots had no or poor roots under the same light and temperature conditions. Each box contained shoots derived from a single embryo. When the plantlets were about 8-10 cm tall with healthy roots, they were transferred to pots with Universal Mix (Strong-Lite, Seneca, Ill.) in a greenhouse.

### Example 22

### Generation of Transgenic Barley

(1) Cloning of Barley Homologs of AtRanBP1b

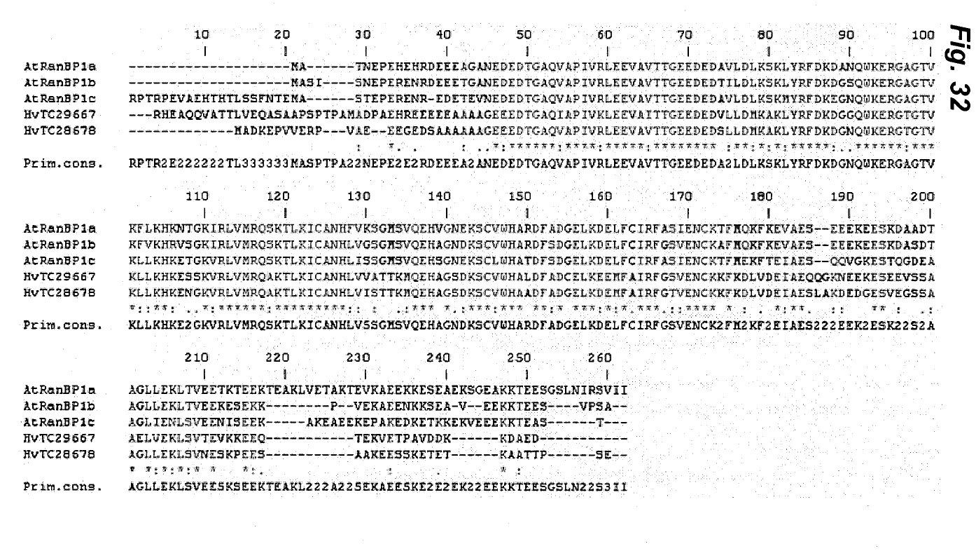

Barley homologs, HvTC29667 and HvTC28678, of AtRanBP1b were found by BLAST searching a cDNA database supported by TIGR as described for the cloning of soybean homologs (see Example 18). FIG. 32 shows their amino acid sequence alignment.

(2) Generation of Barley Expressing in Sense or Antisense Orientation Arabidopsis or Barley Homologs of AtRanBP1b

(2.1) Construction of the Fusion Plasmids Containing AtRanBP1a, AtRanBP1b, AtRanBP1c, Antisense AtRanBP1a, Antisense AtRanBP1b, Antisense AtRanBP1c, HvTC29667, Antisense HvTC29667, HvTC28678, Antisense HvTC28678

DNA fragments containing AtRanBP1a, AtRanBP1b, AtRanBP1c, antisense AtRanBP1a, antisense AtRanBP1b, antisense AtRanBP1c, HvTC29667 (SEQ.ID.NO:54), antisense HvTC29667 (SEQ.ID.NO:55), HvTC28678 (SEQ.ID.NO:56), and antisense HvTC28678 (SEQ.ID.NO:57) were PCR amplified using sequence-specific primers for each case and were inserted into the pSB505 vector as described for rice transformation above.

(2.2) Generation of Transgenic Sorghum Plants

Expression constructs described above were transformed into Hordeum vulgare L. cv. Clipper using an Agrobacterium-mediated transformation as described below.

(a) Preparation of Immature Embryos

Spikes of barley were harvested when the immature embryos were between 1.5 mm and 2.5 mm in length. The developing caryopses were sterilized for 10 min in a solution of sodium hypochlorite containing 1% w/v chlorine, and then rinsed four times in sterile, distilled water. Immature embryos were excised from the young caryopses, and the embryonic axis was removed with a sharp scalpel blade. The explants were placed scutellum-side up on callus induction medium. Embryos were incubated at 24° C. in the dark during cocultivation and subsequent periods of culture.

(b) Transformation

Agrobacterium tumefaciens AGL1 (pDM805) was grown from a single colony in MG/L medium supplemented with 20 mg/L rifampicin and 5 mg/L tetracycline, for 40 h at 27° C. MG/L was prepared as a liquid broth with mannitol 5 g/L, 1-glutamic acid 1 g/L, KH₂PO₄ 250 mg/L. NaCl 100 mg/L, MgSO₄-7H₂O 100 mg/L, biotin 1 mg/L, tryptone 5 g/L, yeast extract 2.5 g/L, with pH adjusted to 7.0. A standard inoculum was prepared by adding 200 μL of culture 200 μL of 15% aqueous glycerol in an eppendorf tube, and kept at room temperature for 6 hours before being transferred to −80° C. A full strength inoculum, approximately 2.8×10⁹ bacterial cells ml⁻¹, was obtained by growing the standard inoculum in 10 ml of MG/L for 24 h at 27° C. Immature embryos, the day after isolation, were injured by shooting the scutellum surface. Fifty to 70 embryos were used per shot, with 0.1 mg or 0.6 mg of gold particles (1.0, BioRad, Regents Park, Australia), using a Bio-Rad PDS-1000 Biolistic device with a 110 p.s.i rupture disc. Following shooting, the embryos were immersed in a full strength Agrobacterium suspension and then immediately transferred, without rinsing, with the scutella surface placed in contact with the callus induction medium. Plates were incubated at 24° C. in darkness for 2 or 3 days. After the co-cultivation, embryos were transferred directly to callus induction medium supplemented with 3 mg/L biolaphos and 150 mg/L Timentin TM. The selection process, as described by Wan and Lemaux (1994), was used for up to 8 weeks. Resistant embryogenic callus lines were transferred to FHG medium (Hunter, 1998) supplemented with 1 mg/L BA, 3 mg/L bialaphos, and solidified with 3 g/L phytagel (Sigma), and incubated at 24° C. under fluorescent lights (16 h/day). Regenerating plantlets were transferred to hormone-free callus induction medium with 1 mg/L biolaphos. After development of a root system, plantlets were transferred to soil and placed in growth cabinets set at 18° C. 8 h day/13° C. night for 4 weeks, then transferred to 16 h day cabinets. Regenerants grew to maturity and self-pollinated.

Alternatively, barley (Hordeum vulgare L. cv. Clipper) was subjected to Agrobacterium-mediated transformation using the Agrobacterium tumefaciens LBA4404 strain. AtRanBP1b in sense and antisense orientations were inserted using the pLBJ21 binary vector, with a CaMV35S promoter and a NPT II selection marker. Explants were prepared by pre-cultivation for three days of scutellar tissues from 1.4-1.6 mm embryos of barley on a modified L1 basal medium. Protoplast isolation was performed according to a standard procedure used for barley cell suspensions after vacuum infiltration of scutellar tissues with cell wall digesting enzymes. Protoplasts were embedded in Na-alginate. Protoplasts were cultured in liquid L3 medium for two weeks. To induce embryogenesis and regenerate plants, macroscopic calli were picked from Na-alginate, washed and subcultured onto embryogenesis induction medium, based on L1. Embryogenic explants were further subcultured for 4-5 weeks for shoot induction and plant regeneration (3-4 months after protoplasts isolation). Regenerants were grown to maturity in pots and fertile plants were recovered. Genetic transformation was confirmed by Genomic PCR, RT-PCR, Southern and Northern blot analysis.

### Example 23

### Generation of Transgenic Lettuce

(I) Cloning of a Lettuce Homolog of AtRanBP1b

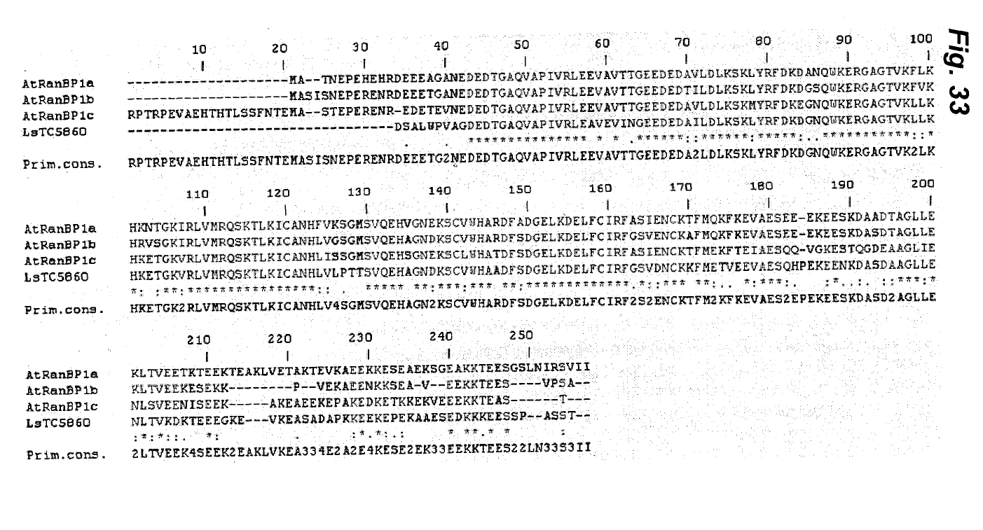

A lettuce homolog, LsTC5860, of AtRanBP1b was found by BLAST searching a cDNA database supported by TIGR as described for the cloning of soybean homologs (see Example 18). FIG. 33 shows their amino acid sequence alignment.

(2) Generation of Lettuce Expressing Sense or Antisense Orientation of Arabidopsis or Lettuce Homologs of AtRanBP1b

(2.1) Construction of the Fusion Plasmids Containing AtRanBP1a, AtRanBP1b, AtRanBP1c, Antisense AtRanBP1a, Antisense AtRanBP1b, Antisense AtRanBP1c, LsTC5860, Antisense LsTC5860

DNA fragments containing AtRanBP1a, AtRanBP1b, AtRanBP1c, antisense AtRanBP1a, antisense AtRanBP1b, antisense AtRanBP1c, LsTC5860 (SEQ.ID.NO:58) and antisense LsTC5860 (SEQ.ID.NO:59) were PCR amplified using sequence-specific primers and were inserted into pLBJ21.

(2.2) Generation of Lettuce Transgenic Plants

Expression constructs described above were transformed into lettuce cotyledons using Agrobacterium-mediated transformation as described below.

(a) Seeds were surface-sterilized by immersion in a 10% (v/v) solution of bleach (0.5% sodium hypochlorite) for 30 mins, followed by thorough rinsing with at least three changes of sterile distilled water.

(b) The seeds were sown onto 20 ml aliquots of germination medium contained in 9 cm diameter petri dishes with approximately 40 seeds per dish. The germination medium consisted of 1/2 MS salts and vitamins with 10 g/L sucrose, lacks growth regulators, and is semisolidified with 8 g/L agar, pH 5.8. The dishes incubated for 7 days at 23° C.±2° C. with a 16 h photoperiod provided by daylight fluorescent tubes, giving an irradiance of 200 μmol m⁻² s⁻¹.

(c) After 7 days, the cotyledons were excised from the seedlings and wounded on their abaxial surface with a scalpel blade, with shallow cuts about 1 mm apart at right angles to the midrib. The wounded cotyledons were ready for inoculation with Agrobacterium.

(d) Bacterial culture was in LB broth (100 ml in conical flasks). The liquid medium also required supplementation with appropriate antibiotics (e.g., 40 mg/L rifampicin, 50 mg/L kanamycin sulfate, and 2 mg/L tetracyclin hydroride). Liquid cultures were incubated for 16 h in the dark on a horizontal rotary shaker and grown to an OD600 of 1.1-1.6, before being used to inoculate the excised cotyledons.

(e) Bacterial cultures are diluted 1:1 or 1:10 (v/v) with liquid Uchimiya and Murashige (UM) medium containing MS salts and vitamins at full strength supplemented with 30 g/L sucrose, 2 g/L casein hydrolysate, 2 mg/L 2,4-dichlorophenoxyacetic acid (2,4-D), 0.23 mg/L Kinetin, 9.9 mg/L thiamine HCl, 9.5 mg/L pyridoxine HCl, and 4.5 mg/L nicotinic acid, pH 5.8.

(f) Excised cotyledons were floated with their scarred surfaces in contact with the 1:10 dilution of Agrobacterium for 10 min or dipped (2-3 s) in a 1:1 dilution of the bacterial suspension. Controls were treated with liquid UM medium, but without bacteria.

(g) Twenty-milliliter aliquots of agar-solidified (0.8% w/v) UM medium were dispensed into 9 cm petri dishes and the surface of the medium covered with a sterile filter paper.

(h) Cotyledons were blotted dry on sterile filter paper and placed with their wounded surfaces in contact with the filter paper overlying the UM medium or sterile water. The cotyledons were positioned in each dish so as to keep the explants flat.

(i) The explants were incubated for 2 days under the same conditions used for germinating seeds.

(j) The cotyledons were transferred to 20 ml aliquots of MS-based shoot regeneration medium containing 30 g/L sucrose, 0.04 mg/L naphthalene acetic acid (NAA), 0.5 mg/L 6-benzyl aminopurine (BAP), that had been semisolidified with 0.8% agar, pH 5.8. The shoot initation medium used for Agrobacterium-inoculated cotyledons also contains 500 mg/L carbenicillin, 100 mg/L cefataxime, and either 50 or 100 mg/L kanamycin sulfate. Explants were subcultured three times on this medium at 14 day intervals.

(k) Explants that were producing callus were transferred individually to 175 ml capacity screw capped glass jars, each containing 40 ml of shoot initiation medium. Carbenicillin was omitted at this stage used to the culture cotyledons inoculated with Agrobacterium. Shoots normally appeared from cultured cotyledons about 35 days after inoculation with Agrobacterium, but this interval varied by cultivar.

(l) Regenerated shoots were excised when approximately 1 cm in height and transferred individually to 175 ml jars containing 40 ml aliquots of MS agar medium lacking growth regulators but with 30 g/L sucrose, pH 5.8. Shoots from Agrobacterium inoculated cotyledons are maintained on MSO agar medium 100 mg/L kanamycin sulfate in order to maintain selective pressure on the transgenic shoots.

(m) Rooted green plants, lacking any signs of bleaching, were removed from their containers; their roots were washed free of culture medium, and individual plants were transferred to plastic pots, the latter containing a mixture of Levington M3 compost, John Innes No.3 compost, and Perlite (3:3:2 by volume).

### Example 24

### Generation of Transgenic Canola

### (1) Construction of the Fusion Plasmids Containing AtRanBP1a, AtRanBP1b, AtRanBP1c, Antisense AtRanBP1a, Antisense AtRanBP1b, Antisense AtRanBP1c.

DNA fragments containing AtRanBP1a, AtRanBP1b, AtRanBP1c, antisense AtRanBP1a, antisense AtRanBP1b, and antisense AtRanBP1c were PCR amplified using sequence-specific primers and were into a pLBJ21 vector.

(2) Generation of Canola Transgenic Plants

Expression constructs described above were transformed into hypocotyls from Brassica napus L. cv. Cascade cotyledons using Agrobacterium-mediated transformation as described below (see also Altenbach et al., Plant Mol Bio. 18:235-245 (1992)).

(a) Sterilization

Seeds of winter canola were surface-sterilized by soaking in 70% ethanol for 1 min followed by a 20-30 min immersion in 1% sodium hypochlorite solution containing 0.1% Tween 80 with continuous agitation. The seed was then washed three times 1/10 strength agar solidified medium without plant growth regulators, and allowed to germinate at 24-28° C. with a 16 h day length and cool white fluorescent illumination of approximately 40 μE m⁻² s⁻¹. Seven days after sowing, the hypocotyls were divided into 5-10 mm explants with a surgical blade.

(b) Agroinfection

Agrobacterium tumefaciens LBA4404 containing one of the above plasmids was grown to log phase in Min A medium containing 1 μg/ml tetracycline and then pelleted by centrifugation at 6000×g for 10 min.

(c) Co-Cultivation

The bacteria were resuspended at a concentration of 5×10⁸ bacteria/ml in Min A supplemented with 100 μM acetosyringone. Freshly prepared hypocotyl explants were dipped individually in the bacterial suspension and plated on agar-solidified callus induction medium (I-medium) containing MS salts, B5 vitamins, 30 g/l sucrose, 18.2 μl mannitol, 0.2 mg/l 2,4-D, 3.0 mg/l kinetin and 590 mg/l MES, pH 5.7. Excess inoculum was dried down onto the explant in a laminar flow hood. Plates containing the explants were then sealed with Micropore (3M) surgical tape and cultured for 3 days at 22-24° C. with a 16 hours day length and cool white fluorescent illumination of 60-80 μE m⁻² s⁻¹.

(d) Selection

After co-cultivation, the explants were washed in liquid medium containing 500 mg/ml cefotaxime and 200 μg/ml vancomycin for six hours with continuous gyratory agitation (20-40 rpm) and at least one medium change. Explants were then transferred to agar-solidified I-medium containing 500 μg/ml cefotaxime and 200 μg/ml vancomycin. After three to four days of culture under the same environmental conditions as were used for cocultivation, the explants were transferred to agar-solidified I-medium containing 200 μg/ml vancomycin and 100 μg/ml kanamycin sulfate. Small white calli became visible on the ends of the hypocotyl explants after two weeks of additional culture.

(e) Regeneration

Calli produced from the explants were plated on regeneration medium (R-medium) which contained K3 salts, B5 vitamins, 10 g/l sucrose, 2 mg/l zeatin, 0.1 mg/l IAA, 290 mg/l MES, pH 5.7 solidified with 4 g/l type 1 agarose. The same temperature and light regimens were used for regeneration as were used for co-cultivation and selection. Calli were plated on fresh medium every two weeks. Shoots generally appeared within two weeks of transfer to R-medium although prolonged culture was required to elicit a shoot response in some calli. When shoots reached 1-2 cm in length, they were excised from the attached callus, dipped in Rootone, and placed in sterile vermiculite in Magenta CP4 vessels. The vermiculite was kept moist by occasional watering with a solution of 1/2 strength MS salts, 10 g/l sucrose. Plants with substantial root systems were transferred to 10 cm pots containing a soil mixture of 2 parts Redi Earth (W. R. Grace Co.), 1 part Supersoil (Rod McLellan Co.) and 1 part Perliter (Redco & Co.).

Regenerated plants were vernalized at 4° C. with a 12 h day length and cool white fluorescent illumination at approximately 40 μm⁻² s⁻¹ for a minimum of seven weeks.

### Example 25

### Generation of Transgenic Sunflower

(1) Construction of the Fusion Plasmids Containing AtRanBP1a, AtRanBP1b, AtRanBP1c, Antisense AtRanBP1a, Antisense AtRanBP1b, Antisense AtRanBP1c.

DNA fragments containing AtRanBP1a, AtRanBP1b, AtRanBP1c, antisense AtRanBP1a, antisense AtRanBP1b, and antisense AtRanBP1c were PCR amplified using sequence-specific primers for each case and were inserted into the pLBJ21 vector.

(2) Generation of Transgenic Sunflower Plants

The expression constructs described above were transformed into embryos of Pioneer hybrid 6440 using Agrobacterium-mediated transformation as described below.

(a) Sterilization

Mature sunflower seeds (Helianthus annuus L.) were dehulled using a single wheat-head thresher. Seeds were surface sterilized for 30 min in a 20% Clorox bleach solution with the addition of two drops of Tween-20/50 ml of solution. The seeds were rinsed twice with sterile distilled water.

(b) Pre-Culture

Seeds were soaked in distilled water for 60 min following the surface sterilization procedure. The cotyledons of each seed were then broken off, producing a clean fracture at the plane of the embryonic axis. Following excision of the root tip, the explants were bisected longitudinally between the primordial leaves. The two halves were placed, cut surface up, on GBA medium containing of MS salts, Shepard's vitamin additions, 40 mg/l adenine sulfate, 30 g/l sucrose, 0.5 mg/l 6-BAP, 0.25 mg/l IAA, 0.1 mg/l GA3, pH 5.6, and 8 g/l Phytagar.

(c) Microinjectile Bombardment

The explants were subjected to microinjectile bombardment prior to Agrobacterium treatment. Thirty to forty explants were placed in a circle at the center of a 60×20 mm plate for this treatment. Approximately 4.7 mg of 1.8 μm tungsten microinjectiles were re-suspended in 25 mL of sterile TE buffer (10 mM TRIS-HCl, 1 mM EDTA, pH 8) and 1.5 mL aliquots were used per bombardment. Each plate was bombarded twice through a 150 mL Nytex screen placed 2 cm above the samples in a PDS 1000 particle acceleration device.

(d) Agroinfection

Disarmed Agrobacterium tumefaciens strain EHA101 transformed with a binary T-DNA PHP 158 plasmid was used in all transformation experiments. Bacteria for plant transformation experiments were grown overnight (at 28° C. and 100 rpm continuous agitation in liquid YEP medium (10 g/l yeast extract, 1-g/l Bactopeptone and 5 g/l NaCl, pH 7.0) with the appropriate antibiotics required for bacterial strain and binary plasmid maintenance. The suspension was used when it reached an OD600 of 0.5 in an inoculation medium comprised of 12.5 mM MES (PH 5.7), 1 mg/l NH₄Cl and 0.3 g/l MgSO₄. Freshly bombarded explants were placed in an Agrobacterium suspension, mixed and left untransferred to GBA medium and co-cultivated curt surface down at 26° C. for 18-h days.

(e) Selection and Regeneration

After 3 days of co-cultivation, the explants were transferred to 374B (GBA medium lacking growth regulators and having a reduced sucrose level of 1%) supplemented with 250 mg/l cefotaxime and 25, 50, 100 or 200 mg/l kanamycin sulfate. The explants were cultured for 2-5 weeks on selection and then transferred to fresh 374B medium lacking kanamycin for 1-2 weeks of continued development. Explants with differentiating, antibiotic resistant areas of growth that had not produced shoots suitable for excision were transferred to GBA medium containing 250 mg/l cefotaxime for a second 3-day phytohormone treatment. Leaf samples from green, kanamycin-resistant shoots were assayed for the presence of NPTII activity. Those shoots that failed to exhibit NPTII activity were discarded. NPTII-positive shoots were grafted to Pioneer hybrid 6440 in vitro grown sunflower seedling rootstock as follows. Surface sterilized seeds were germinated in 48-0 medium (half strength MS salts, 0.5% glucose, 0.3% gelrite, pH 5.6) and grown under the conditions described for explant culture. The upper portion of the seedling was removed, a 1-cm vertical slice was made in the hypocotyl and the transformed shoot inserted into the cut. The entire area was wrapped with parafilm to secure the shoot. Grafted plants were transferred to soil following 1 week of in vitro culture. Grafts in soil were maintained under high humidity conditions followed by a slow acclimatization to the greenhouse environment.

### Example 26

### Transformation of Onion (Monocot)

Onion (Allium cepa L.) was subjected to Agrobacterium-mediated transformation using the Agrobacterium tumefaciens LBA4404 strain. AtRanBP1b and AtRanBP1c were inserted in sense and antisense orientations using a pLBJ21 binary vector. Immature zygotic embryos were collected and vortexed for 30 s. Embryos were cut into 1 mm lengths for vacuum infiltration (20 Hg, for 30 min), followed by cocultivation on P5 innoculation medium for 6 days. Embryo pieces were cultured in the dark under the same conditions for 2 weeks. Putative transgenic tissue then was transferred to regeneration medium. Shoots were then transferred to rooting medium. Regeneration of transformants was on media containing IAA/BA. Genetic transformation was confirmed by genomic PCR, RT-PCR, Southern and Northern blot analysis.

### Example 27

### Stable Transformation of Pine Trees

The transformation protocol is based on an adaptation of the method presented in Cerda et al., Plant Cell, Tissue and Organ Culture 70:251-257, 2002. For sample collection, immature open-pollinated cones are treated for 10 min with 10% commercial detergent, for 10 min with 20% commercial bleach, then washed three times (10 min each) in sterile distilled water, and then the seed coat is removed. Induction is in induction medium (IM: Westvaco WV5 medium, Duchefa, with 3% sucrose, 3 mg/l 2,4-D, 0.5 mg/l BA, and 500 mg/l casein hydrolysate, and 0.8% Agar as a solidifying agent) in the dark for 4-6 weeks. Embryogenic tissue is maintained in proliferation medium (IM also containing 100 mg/l L-glutamine, 522 mg/l L-arginine, 8 mg/l L-alanine and 7 mg/l L-proline), and sub-cultured to fresh media every two weeks. For transformation, Agrobacterium cells are prepared to an OD600 of 0.6, re-suspended in liquid PM medium, and put in Petri dishes. Embryogenic tissue is then added and inoculated for three minutes, then dried on filter paper and placed on PM medium. Co-cultivation follows for 2 days in the dark. Then, the tissues are washed with 250 mg/l cefotaxime solution and transfered to PM supplemented with 250 mg/l cefotaxime and 100 mg/l kanamycine. Maturation is in maturation medium (MM: Westvaco WV3 medium, Duchefa, with 3% sucrose, 4500 mg/l L-glutamine, 2000 mg/l L-asparagine, 783 mg/l arginine 32 mg/l L-alanine and 28 mg/l L-proline, and 90 μM racemic abscisic acid (ABA), and 0.45% Gelrite as a solidifying agent). Shoot generation medium is WV3 medium, Duchefa, with 2% sucrose, 2.29 mg L-glutamine 5% activated charcoal and 0.8% Agar as a solidifying agent. Root generation medium is half strength MS basal medium, Duchefa. The pH of all media is about 5.7-5.8.

### Example 28

### Walnut Somatic Embryo Transformation

The transformation protocol is based on an adaptation of the methods presented in Plant Cell Reports 19:881-887, 2000 and Biotechnology in Agriculture and Forestry 39:345-357 (1997). Immature fruits are sterilized for 20-30 seconds with 70% EtOH and for 20-30 seconds with 1% sodium hypochlorite, and then rinsed three times with sterile distilled water. Then, the seed coat and cotyledon are removed. Embryogenic tissue is induced in the dark in induction medium: MS medium, Duchefa, with % sucrose, 250 mg/l L-glutamine, 0.05 μM IBA, 4.4 μM BA and 9.3 μM kinetin and 0.7% Agar as a solidifying agent. For transformation, Agrobacterium cells are prepared to an OD600 of 0.6, re-suspended in liquid MS medium, and put in Petri dishes. Embryogenic tissues is then added and inoculated for ten minutes in Agrobacterium suspension, then dried on filter paper and placed on solid MS basal medium containing 100 μM acetosyringone. Co-cultivation follows for 2 days in the dark. Then, tissues are washed with 250 mg/l cefotaxime solution, and transferred to IM supplemented with 250 mg/l cefotaxime and 100 mg/l kanamycine. The tissues are then dessicated on dry microtiter plates for 2 weeks at 4° C. For shoot generation, the tissues are re-hydrated on MS basal medium containing 5 g/l charcoal in the dark for 2 weeks, then put in shoot generation medium (MS medium, Duchefa,), no growth regulator, and 0.8% Agar as a solidifying agent. Plates are placed under white light. Rooting is in root generation medium (Half strength MS basal medium, Duchefa).

### Example 29

### Transformation Protocol for Hybrid Cottonwoods

Transformation is based on an adaptation of the method presented in Han et al., Transgen. Res. 6: 415-420. For growth room-grown materials, greenwood stems or young leaves are sterilized in 20% Clorox solution for 20 min followed by 5 washes with sterilized distilled water. For tissue culture materials, stem internodes and leaves are harvested at the end of bi-monthly subculture. Agrobacterium cells carrying a binary vector are grown overnight at 25° C. in liquid Luria Bertani (LB: 16 g/l Bacto-tryptone, 8 g/l Bacto-yeast extract and 5 g/l NaCl, adjusted to pH 7.0) medium supplemented with appropriate antibiotics. The cells are collected by centrifugation at 2,560 rpm (1292 RCF) for 30 min and resuspended in induction medium (IM: MS salts and vitamins+10 uM AS+10 mM galactose+1.28 mM MES; pH 5.0). The cells are centrifuged as above and resuspended in the IM to a density of OD600=0.3-0.4, induced with shaking (50-100 rpm) for 1 hr at room temperature. Explants, with or without pre-culture for 2-7 days on CIM (MS+0.5 uM BA+0.5 uM zeatin+5 uM NAA+5 uM 2,4-D+0.3% Phytoagar+0.1% Gelrite+1.28 mM MES; pH 5.8), are soaked for 10-20 min in the bacterial suspension under 0.6 bar vacuum, and incubated on a shaker (50 rpm) for 1-2 hrs at room temperature. The inoculated explants are co-cultivated on CIM at 19-25° C. in the dark for 2-3 days. Explants are washed four times with double-distilled water and once with wash solution (WS: 1/2 MS salts and vitamins+250 mg/l ascorbic acid+1 uM NAA+1 uM BA+1 uM 21P+500 mg/l cefotaxime; pH 5.8). Explants are cultured for 10-30 days in the dark on CIM supplemented with 500 mg/l cefotaxime and 50 mg/l kanamycin. Shoot regeneration is induced on SIM (MS+10 uM BA+10 uM zeatin+1 uM NAA+0.3% Phytoagar+0.1% Gelrite+1.28 mM MES; pH 5.8) supplemented with 100 mg/l kanamycin for several weeks to months. Regenerated shoots are screened for kanamycin resistance by rooting on 1/2 MS medium supplemented with 0.5 uM IBA and 25 mg/l kanamycin.

### Example 30

### Ploidy Determination

A “big plant phenotype” is one of biological characteristics often observed in tetraploid plants. Therefore, the ploidy level of transgenic plants that have big plant phenotype was determined.

(1) Transgenic Arabidopsis Expressing Antisense AtRanBP1b

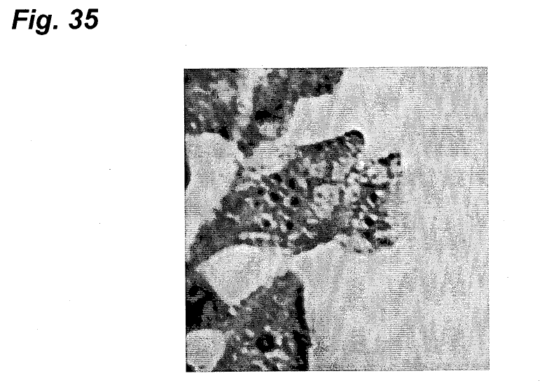

The chromosomes of a pollen mother cell were stained with Giemsa dye and its chromosomes were counted. Wild type Arabidopsis has 5 chromosomes, and as shown in FIG. 35, the previously described pLBJ21/antisense AtRanBP1b-5 plant has 10 chromosomes.

(2) Transgenic Tobacco Expressing Antisense AtRanBP1b

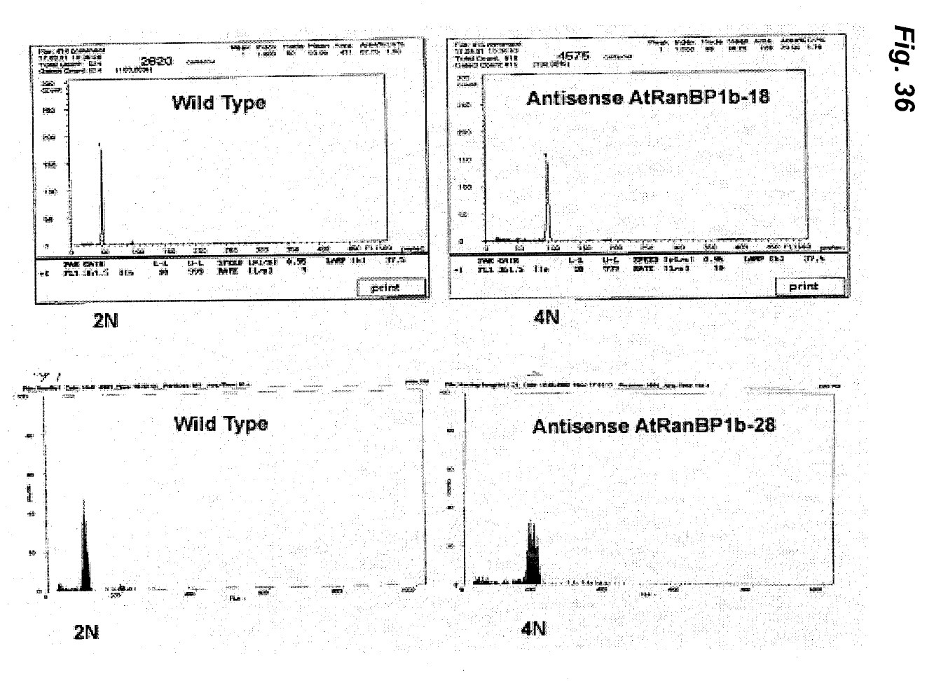

Leaf punches of transgenic tobacco plants expressing antisense AtRanBP1b and exhibiting a big plant phenotype (tabulated in Table 11, Table 12, and FIG. 7) were prepared and their ploidy level checked using a flow cytometer. To count the chromosomes, approximately 0.5 cm² leaf was taken from 40 day-old WT and transgenic tobacco plants and then chopped with a sharp razor blade in extraction buffer Cystain UV precise (Partec, Germany). The nuclei suspension extracted was then filtered through a 30 μm disposable filter. After addition of a DNA staining buffer, DNA content was analyzed using a Partec flow cytometer. FIG. 36 shows the ploidy level as compared to that of leaves from wild type plants in two representative independent transgenic tobacco plant leaves. As the results show, the transgenic tobacco plants are tetraploid.

As the above results show, plant Ran and AtRanBP1c are crucial in the regulation of auxin-induced cell cycle progression. Transgenic plants with altered levels of Ran or AtRanBP1c have increased auxin sensitivity for mitotic root cells. This modification results in long-rooted transgenic plants. AtRanBP1b is shown to be an important regulator of meiotic and mitotic progression, and revealed that AtRanBP1b controls cell cycle progression from metaphase to interphase in dividing plant cells. Transgenic plants with an altered level of AtRanBP1b have a big plant phenotype and produce a high product yield. Transgenic plants with increased body and/or seed size and/or increased root length can be obtained by overexpressing Ran and/or RanBPs or by suppressing their expressions. Increased yield is seen with crops transformed with recombinant expression vectors of the present invention, thus the vectors can be used for development of high yield, super-productive species of plants. Transgenic plants in which Ran-mediated cellular processes are modified show one or more of the phenotypes of long root, big-sized seed, and big body. Also, these plants often have the desirable molecular phenotype of polyploidy. Thus, superior species can be developed by the method for generating transgenic plants modified in Ran-mediated cellular processes of the present invention as mentioned above.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

What is claimed is:

1. A method of making a polyploid plant, said method comprising the steps of:
transforming a plant cell of interest with an expression construct compring a sense or an antisense nucleic acid encoding a Ran or Ran-binding protein;
generating a transgenic plant from said transformed plant cell; and
growing said transgenic plant under conditions whereby said nucleic acid is expressed and a polyploid transgenic plant is obtained.

2. The method according to claim 1, wherein said plant is a cereal plant.

3. The method according to claim 2, wherein said cereal plant is selected from the group consisting of rice, wheat and corn.

4. The method according to claim 2, wherein said step of transforming is bombarding one or more tissue comprising said plant cell with a plurality of particles comprising said expression construct.

5. The method according to claim 1, wherein said plant is a dicotyldenous plant.

6. The method according to claim 5, wherein said dicotyldenous plant is selected from the group consisting of cotton, soybean, sunflower, canola and tobacco.

7. The method according to claim 5, wherein said step of transforming is using an Agrobacterium vector.

8. The method according to claim 1, wherein said plant is a tree.

9. The method according to claim 8, wherein said tree is selected from the group consisting of poplar, eucalyptus, loblolly pine, poplar and sweet gum.

10. The method according to claim 11, wherein said plant is monocotylendenous.

11. The method according to claim 10, wherein said monocotylendenous plant is selected from the group consisting of sorghum, barley and sugarcane.

12. The method according to claim 1, wherein said plant is a grass.

13. The method according to claim 1, wherein said plant is a root crop.

14. The method according to claim 13, wherein said root crop is selected from the group consisting of ginseng and cassaya.

15. A method for making a tetraploid plant cell, said method comprising the steps of:
transforming a plant cell of interest with one or more expression construct comprising as operably linked components a transcription initiation region, an open reading frame encoding a Ran or Ran-binding protein, and a transcription termination region, wherein at least one of said components is other than a nucleic acid sequence that occurs naturally with other of said components; and
growing said plant cell under conditions whereby said open reading frame is expressed and a tetraploid plant cell is obtained.

16. The method according to claim 15, wherein said step of growing is in tissue culture.

17. The method according to claim 15, wherein plant cell of interest produces a secondary metabolite of interest.

18. The method according to claim 15, wherein said secondary metabolite of interest is a plant pharmaceutical.

19. The method according to claim 18, wherein said plant pharmaceutical is selected from the group consisting of taxol, genistein, diadzein, codeine, morphine, quinine, shikonin, ajmalacine and serpentine.

20. The method according to claim 15, further comprising the step of:
generating a plant from said tetraploid plant cell.

21. The method according to claim 29, wherein said plant is selected from the group consisting of tomato, rice, tobacco, cotton, pepper, potato, maize, soybean, wheat, barley, alfalfa, sorghum, lettuce, canola, sunflower, onion, pine, walnut, and cottonwood.

22. A plant obtained according to the method of claim 20.

23. A tetraploid tomato plant obtained by expression of a sense expression construct comprising AtRanBP1c.

24. A nucleic acid comprising:
a sense or an antisense open reading frame encoding a Ran-binding protein obtainable from a plant selected from the group consisting of rice, soybean, cotton, corn, tobacco, potato, wheat, alfalfa, sorghum, barley, and lettuce.

25. The nucleic acid according to claim 24, wherein said Ran-binding protein is a homolog of AtRanBP1b.

26. The nucleic acid according to claim 24, wherein said homolog is selected from the group consisting of OsTC84425 (SEQ.ID.NO: 60), antisense OsTC84425 (SEQ.ID.NO: 61), NtRanBP1 (SEQ.ID.NO: 36), antisense NtRanBP1 (SEQ.ID.NO: 37), GhRanBP1-1 (SEQ.ID.NO: 20), antisense GhRanBP1-1 (SEQ.ID.NO: 21), GhRanBP1-2 (SEQ.ID.NO:22), antisense GhRanBP1-2 (SEQ.ID.NO:23), GhTC9172 (SEQ.ID.NO:24), antisense GhTC9172 (SEQ.ID.NO:25), GhTC11016 (SEQ.ID.NO:26), antisense GhTC11016 (SEQ.ID.NO:27), GhTC9086 (SEQ.ID.NO:28), antisense GhTC9086 (SEQ.ID.NO:29), GhTC9678 (SEQ.ID.NO:30), antisense GhTC9678 (SEQ.ID.NO:31), StTC45414 (SEQ.ID.NO:38), antisense StTC45414 (SEQ.ID.NO:39), StTC43808 (SEQ.ID.NO:40), antisense StTC43808 (SEQ.ID.NO:41), ZmTC140385 (SEQ.ID.NO:32), antisense ZmTC140385 (SEQ.ID.NO:33), ZmTC131044 (SEQ.ID.NO:34), antisense ZmTC131044 (SEQ.ID.NO:35), GmTC120064 (SEQ.ID.NO:16), antisense GmTC120064 (SEQ.ID.NO:17), GmTC120070 (SEQ.ID.NO:18), antisense GmTC120070 (SEQ.ID.NO:19), TaTC45963 (SEQ.ID.NO:42), antisense TaTC45963 (SEQ.ID.NO:43), TaTC57823 (SEQ.ID.NO:44), antisense TaTC57823 (SEQ.ID.NO:45), MtTC43554 (SEQ.ID.NO:46), antisense MtTC43554 (SEQ.ID.NO:47), MtTC45204 (SEQ.ID.NO:48), antisense MtTC45204 (SEQ.ID.NO:49), SbTC34477 (SEQ.ID.NO:50), antisense SbTC34477 (SEQ.ID.NO:51), SbTC41149 (SEQ.ID.NO:52), antisense SbTC41149 (SEQ.ID.NO:53), HvTC29667 (SEQ.ID.NO:54), antisense HvTC29667 (SEQ.ID.NO:55), HvTC28678 (SEQ.ID.NO:56), antisense HvTC28678 (SEQ.ID.NO:57), LsTC5860 (SEQ.ID.NO:58) antisense LsTC5860 (SEQ.ID.NO:59).

25. A recombinant plant cell comprising:
a sense or antisense expression construct comprising AtRanBP1b, wherein said plant cell is selected from the group consisting of Oryza sativa, Nicotiana tabacum, Gossypium hirsutum, Capsicum annuum, Solanum tuberrosum, Zea mays, Glycine mas, Triticum aestiuum, Medicago truncatula, Sorghum bicolor, Hordeum vulgar, Lactuca saliva, Brassica napus, Helianthus annus, and Allium cepa.

28. A recombinant plant comprising:
a recombinant plant cell according to claim 27.

29. The recombinant plant cell according to claim 27 or claim 28, wherein said recombinant plant cell is selected from the group consisting of Oryza sativa cv. Nakdong, Nicotiana tabacum cv Xanthi.

30. A recombinant plant tissue or plant part obtained from a recombinant plant according to claim 28, wherein at least one of size or number of said plant tissue or plant part is increased as compared to that of a wild type plant.

31. The plant tissue according to claim 30, wherein said plant tissue is a seed.

32. The plant tissue according to claim 30, wherein said plant is Nicotiana tabacum and said plant tissue is a leaf.

33. An expression construct comprising a nucleic acid according to claim 26.
